(19) United States
(12) Patent Application Publication
(10) Pub. No.: US 2011/0038835 A1
(43) Pub. Date: Feb. 17, 2011

(54) ANILIDES AND ANALOGS AS RHO KINASE INHIBITORS

(75) Inventors: Yangbo Feng, Palm Beach Gardens, FL (US); Philip LoGrasso, Jupiter, FL (US); Thomas Bannister, Palm Beach Gardens, FL (US); Thomas Schroeter, Riviera Beach, FL (US); Xingang Fang, Riviera, FL (US); Yen ting Chen, Palm Beach Gardens, FL (US); Yan Yin, Jupiter, FL (US); Michael P. Smolinski, Port St. Lucie, FL (US); Lei Yao, Tianjin (CN); Bo Wang, Collegeville, PA (US); Hampton Sessions, Orlando, FL (US)

Correspondence Address: SCHWEGMAN, LUNDBERG & WOESSNER, P.A., P.O. BOX 2938, MINNEAPOLIS, MN, 55402, US

(73) Assignee: The Scripps Research Institute, La Jolla, CA (US)

(21) Appl. No.: 12/808,052

(22) PCT Filed: Dec. 18, 2008
(86) PCT No.: PCT/US08/13851

§ 371 Date: Sep. 30, 2010

Related U.S. Application Data
(60) Provisional application No. 61/008,493, filed on Dec. 19, 2007.

(51) Int. Cl. A61K 31/5377; C07D 231/12; A61K 31/415; C07D 471/02; A61K 31/437; C07D 487/04; A61K 31/519; C07D 401/12; A61K 31/4439; C07D 217/00; A61K 31/47; C07D 413/10; C07D 413/14; A61P 9/00; A61P 9/12; A61P 9/10; A61P 25/00; A61P 25/16; A61P 11/06; A61P 19/02; A61P 3/00; A61K 39/395; A61K 33/24; A61K 31/675; A61K 38/21

(52) U.S. Cl. 424/85.6; 548/377.1; 514/406; 546/113; 514/300; 544/280; 514/265.1; 546/122; 546/275.4; 514/341; 546/146; 514/307; 544/140; 514/236.5; 544/128; 514/235.2; 424/141.1; 424/649; 514/79; 424/133.1

(57) ABSTRACT

Compounds useful as Rho kinase inhibitors of formula (I): wherein variable are as defined herein are provided. Methods of treatment of malconditions mediated by Rho kinase, and methods of preparation of the compounds, are also provided.

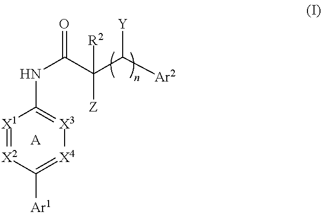

### CROSS-REFERENCE TO RELATED APPLICATIONS

This application claims the priority of U.S. Ser. No. 61/008,493, filed Dec. 19, 2007, which is incorporated herein by reference in its entirety.

### BACKGROUND

Rho kinases, also known as Rho-associated kinases, are serine/threonine kinases that function downstream of Rho which is a low molecular GTP-binding protein. Two Rho kinase isoforms, termed ROCK I and ROCK II, have been identified. The enzymes are believed to be involved in a variety of biological events such as smooth muscle contraction, apoptosis, cell growth, cell migration, cell proliferation, cytokinesis, cytoskeletal control, and inflammation, and to be involved in pathology of various diseases including cardiovascular disease, tumor infiltration, osteogenesis, chondrocyte differentiation and neurogenic pain. See, e.g., H. Satoh, et al., Jpn. J. Pharmacol., 1999, 79, Suppl I, 211, K. Kuwahara, et al., FEBS Lett., 1999, 452, 314-18; N. Sawada, et al., Circulation, 2000, 101, 2030-33; C. Kataoka, et al., Hypertension, 2002, 39(2), 245-50; F. Imamura, et al., Jpn. J. Cancer Res., 2000, 91, 811-16, K. Itoh et al, Nature Medicine, 1999, 5, 221-5, M. Nakajima, et al., Clin. Exp. Pharmacol. Physiol., 2003; 30(7): 457-63; W. Guoyan, et al., J. Biol. Chem., 2004, 279(13), 13205-14; S. Tatsumi, Neuroscience, 2005, 131(2) 491-98.

It is therefore believed that Rho kinase inhibitors have utility in the treatment of diseases and conditions such as hypertension, atherosclerosis, stroke, angina, arterial obstruction, peripheral arterial disease, peripheral circulation disorder, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, multiple sclerosis, diabetes, urinary organ diseases such as overactive bladder (OAB) and benign prostatic hypertrophy (BPH), metastasis, cancer, glaucoma, ocular hypertension, retinopathy, autoimmune disease and viral infection, and myocardial protection.

Various compounds have been described in the literature as Rho kinase inhibitors. See, e.g. WO98/06433; WO00/09162; WO00/78351; WO01/17562; WO02/076976; EP1256574; WO02/100833; WO03/082808; WO2004/009555; WO2004/024717; WO2004/041813; WO2004/108724; WO2005/003101; WO2005/035501; WO2005/035503; WO2005/035506; WO2005/037198; WO2005/058891; WO2005/061463; WO2005/074642; WO2005/074643; WO2005/080934; WO2005/082367; WO2005/082890; WO2005/097790; WO2005/100342; WO2005/103050; WO2005/105780; WO2005/108397; WO2006/044753; WO2006/051311; WO2006/057270; WO2006/058120; WO2006/065946; WO2006/099268; WO2006/072792; WO2006/127587; WO2006/136829; WO2006/136837; WO2007/026920; WO2008/110846; A. Takami, et al., Bioorg. Med. Chem., 2004, 12, 2115-37; M. Iwakubo, et al., Bioorg. Med. Chem., 2007, 15, 350-64; M. Iwakubo, et al., Bioorg. Med. Chem., 2007, 15, 1022-33.

### SUMMARY

The present invention is directed to certain compounds and compositions that are effective Rho kinase inhibitors, to methods of their use in the treatment of diseases for which inhibition of Rho kinase is therapeutically indicated, and to methods for their preparation.

In various embodiments, the invention provides a compound of formula I:

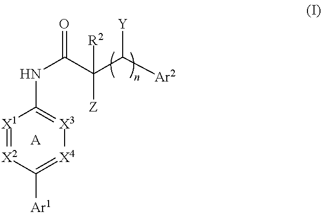

wherein

in ring A comprising each of X¹, X², X³, and X⁴, each of X¹, X², X³, and X⁴ is independently CH, CR¹ or N, provided that no more that two of X¹, X², X³, and X⁴ are N;

R¹ comprises independently at each occurrence F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(4-10 membered)-heterocyclyl substituted with 0-2 R^{a} comprising 1-4 heteroatoms selected from O, S(O)_{q}, and N; or two adjacent R¹ substituents can form a fused phenyl or a 5-6 membered heteroaryl comprising carbon atoms and 1-2 heteroatoms selected from O, S(O)_{q}, and N, and substituted with 0-3 R^{a}, wherein p is 0-4 and q is 0-2;

R is independently at each occurrence H, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (3-10 membered)-cycloalkyl substituted with 0-2 R^{a}, or (3-10 membered)-heterocyclyl substituted with 0-2 R^{a} comprising 1-4 heteroatoms selected from O, S(O)_{q}, and N; or, an NR₂ forms a (3-10 membered)-heterocyclyl substituted with 0-2 R^{a} and comprising 0-1 additional ring heteroatoms selected from N, O, and S(O)_{q};

R^{a} is independently at each occurrence oxo, F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(3-10 membered)-heterocyclyl substituted with 0-2 R^{a} comprising 1-4 heteroatoms selected from O, S(O)_{q}, and N;

Ar¹ comprises a 5- or 6-membered heteroaryl comprising at least one nitrogen atom and 0-3 additional heteroatoms selected from O, S(O)_{q}, and N; when Ar¹ is a 5-membered heteroaryl, a nitrogen atom is disposed one atom away from an atom of the heteroaryl bonded to ring A, and when Ar¹ is a 6-membered heteroaryl, a nitrogen atom is disposed two atoms away from an atom of the heteroaryl bonded to ring A; wherein Ar¹ is optionally fused with phenyl or a 5-6 membered heteroaryl comprising 1-2 heteroatoms selected from O, S(O)_{q}, and N, wherein the fused phenyl or 5-6 membered heteroaryl is substituted with 0-3 R^{a};

provided that when Ar¹ comprises pyrazolyl, pyridyl, or pyrimidyl, then ring A is substituted with at least one R¹ which is other than unsubstituted alkyl;

R^{b} is independently at each occurrence H, (C₁₋₆)-alkyl, (C₃₋₆)-cycloalkyl, (C₁₋₆)alkyl-(C₃₋₆)-cycloalkyl, (3-8 membered)-heterocyclyl, (C₁₋₆)alkyl-(3-8 membered)-heterocyclyl, aryl, heteroaryl, aralkyl, or heteroarylalkyl, wherein any R^{b} other than H is substituted with 0-3 R^{a};

R² is H or (C₁₋₆)-alkyl;

n is 0 or 1;

and when n=1, Y is H,

or Y comprising —CH₂— together with Z comprising —CH₂NR^{b}—, and carbon atoms to which Y and Z are bonded can together form a 5- or 6-membered heterocyclyl ring substituted with 0-3 R^{a}, or

when n=0 or 1, Z comprises NH₂ or OH; and,

Ar² comprises aryl, heteroaryl, or is absent, wherein any aryl or heteroaryl is substituted with 0-3 R^{a},

or when Ar² is aryl or heteroaryl, Ar² together with Z comprising NR^{b} can together form an aryl- or heteroaryl-fused 5- or 6-membered heterocyclyl substituted with 0-3 R^{a};

or any salt, stereoisomer, tautomer, hydrate, solvate, or prodrug thereof.

In various embodiments, the invention provides methods of synthesis of compounds of the invention.

In various embodiments, the invention provides a pharmaceutical composition comprising a compound of the invention and a suitable excipient.

In various embodiments, the invention provides a pharmaceutical combination comprising a compound of the invention and a second medicament.

In various embodiments, the invention provides a method of treatment of a malcondition in a patient comprising administering a therapeutically effective amount of a compound, pharmaceutical composition, or pharmaceutical combination of the invention to the patient at a frequency of administration and for a duration of time sufficient to provide a beneficial effect to the patient.

In various embodments, the inventive method can further comprises administration of an effective a second medicament to the patient at a frequency and for a duration sufficient to provide a beneficial effect to the patient. The second medicament can be an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

In various embodiments, the invention provides a method of treatment of a malcondition in a patient, comprising administering to the patient the pharmaceutical combination of the invention or a pharmaceutical composition comprising the inventive combination in a therapeutically effective amount at a frequency of administration and for a duration of time sufficient to provide a beneficial effect to the patient.

The malcondition can comprise cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof. The malcondition can be one for the treatment of which binding of a ligand to a Rho kinase or inhibition of a bioactivity of a Rho kinase, or both, is medically indicated.

In various embodiments, the invention provides a use of a compound, composition, or combination of the invention in the preparation of a medicament for treatment of a malcondition. The malcondition can be one wherein binding of a ligand to a Rho kinase or inhibition of a bioactivity of a Rho kinase, or both, is medically indicated. The malcondition can include cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the invention provides a compound of the invention for use in treatment of cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the invention provides a compound of any the invention for use in combination with an effective amount of a second bioactive agent in treatment of cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof. The second medicament can be an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

### DETAILED DESCRIPTION

### Definitions

As used in the specification and the appended claims, the singular forms “a,” “an” and “the” include plural referents unless the context clearly dictates otherwise.

As used herein, “individual” (as in the subject of the treatment) means both mammals and non-mammals. Mammals include, for example, humans; non-human primates, e.g. apes and monkeys; cattle; horses; sheep; and goats. Non-mammals include, for example, fish and birds.

The term “Rho-kinase-mediated disease” or “Rho-kinase-mediated disorder” are used interchangeably, and are used to refer to diseases or conditions wherein a Rho-kinase (ROCK) plays a role in the biochemical mechanisms involved in the diseases such that a therapeutically beneficial effect can be achieved by inhibiting a Rho-kinase.

The expression “effective amount”, when used to describe therapy to an individual suffering from Rho-kinase-mediated disorder, refers to the amount of a compound of the invention that is effective to inhibit or otherwise act on a Rho kinase in the individual's tissues wherein the Rho-kinase involved in the disorder is active, wherein such inhibition or other action occurs to an extent sufficient to produce a beneficial therapeutic effect.

“Treating” or “treatment” within the meaning herein refers to an alleviation of symptoms associated with a disorder or disease, or inhibition of further progression or worsening of those symptoms, or prevention or prophylaxis of the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim.

When a substituent is specified to be an atom or atoms of specified identity, “or a bond”, a configuration is referred to when the substituent is “a bond” that the groups that are immediately adjacent to the specified substituent are directly connected to each other by a chemically feasible bonding configuration.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted. When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂CH₂—S(═O)—CH₃, and —CH₂CH₂—O—CH₂CH₂—O—CH₃. Up to two heteroatoms may be consecutive, such as, for example, —CH₂—NH—OCH₃, or —CH₂—CH₂—S—S—CH₃.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and —CH═CH—O—CH₂CH₂—O—CH₃.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl-1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein.

“Halo” as the term is used herein includes fluoro, chloro, bromo, and iodo. A “haloalkyl” group includes mono-halo alkyl groups, and poly-halo alkyl groups wherein all halo atoms can be the same or different. Examples of haloalkyl include trifluoromethyl, 1,1-dichloroethyl, 1,2-dichloroethyl, 1,3-dibromo-3,3-difluoropropyl and the like.

The terms “halo” or “halogen” or “halide” by themselves or as part of another substituent mean, unless otherwise stated, a fluorine, chlorine, bromine, or iodine atom, preferably, fluorine, chlorine, or bromine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

The terms “aryloxy” and “arylalkoxy” refer to, respectively, an aryl group bonded to an oxygen atom and an aralkyl group bonded to the oxygen atom at the alkyl moeity. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R3N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each. Accordingly, any compound substituted with an amino group can be viewed as an amine.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to carbamoyl groups (—C(O)NH₂) and formamide groups (—NHC(O)H). A “carboxamido” group is a group of the formula C(O)NR₂, wherein R can be H, alkyl, aryl, etc.

The term “urethane” (or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein.

A “hydrate” is a compound that exists in a composition with water molecules. The composition can include water in stoichiometic quantities, such as a monohydrate or a dihydrate, or can include water in random amounts.

A “solvate” is a similar composition except that a solvent other that water replaces the water. For example, methanol or ethanol can form an “alcoholate”, which can again be stoichiometic or non-stoichiometric.

“Tautomers” are two forms of a substance differing only by the position of a hydrogen atom in the molecular structures.

A “prodrug” as is well known in the art is a substance that can be administered to a patient where the substance is converted in vivo by the action of biochemicals within the patients body, such as enzymes, to the active pharmaceutical ingredient. Examples of prodrugs include esters of carboxylic acid groups, which can be hydrolyzed by endogenous esterases as are found in the bloodstream of humans and other mammals.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

In various embodiments, the compound or set of compounds, such as are used in the inventive methods, can be any one of any of the combinations and/or sub-combinations of the above-listed embodiments.

### Description

### Compounds of the Invention

In various embodiments, the invention provides a compound of formula I:

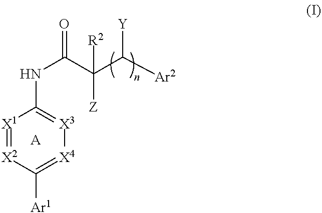

wherein

in ring A comprising each of X¹, X², X³, and X⁴, each of X¹, X², X³, and X⁴ is independently CH, CR¹ or N, provided that no more that two of X¹, X², X³, and X⁴ are N;

R¹ comprises independently at each occurrence F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(4-10 membered)-heterocyclyl substituted with 0-2 R^{a} comprising 1-4 heteroatoms selected from O, S(O)_{q}, and N; or two adjacent R¹ substituents can form a fused phenyl or a 5-6 membered heteroaryl comprising carbon atoms and 1-2 heteroatoms selected from O, S(O)_{q}, and N, and substituted with 0-3 R^{a}, wherein p is 0-4 and q is 0-2;

R is independently at each occurrence 1-1, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (3-10 membered)-cycloalkyl substituted with 0-2 R^{a}, or (3-10 membered)-heterocyclyl substituted with 0-2 R^{a} comprising 1-4 heteroatoms selected from O, S(O)_{q}, and N; or, an NR₂ forms a (3-10 membered)-heterocyclyl substituted with 0-2 R^{a} and comprising 0-1 additional ring heteroatoms selected from N, O, and S(O)_{q};

R^{a} is independently at each occurrence oxo, F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(3-10 membered)-heterocyclyl substituted with 0-2 R^{a} comprising 1-4 heteroatoms selected from O, S(O)_{q}, and N;

Ar¹ comprises a 5- or 6-membered heteroaryl comprising at least one nitrogen atom and 0-3 additional heteroatoms selected from O, S(O)_{q}, and N; when Ar¹ is a 5-membered heteroaryl, a nitrogen atom is disposed one atom away from an atom of the heteroaryl bonded to ring A, and when Ar¹ is a 6-membered heteroaryl, a nitrogen atom is disposed two atoms away from an atom of the heteroaryl bonded to ring A; wherein Ar¹ is optionally fused with phenyl or a 5-6 membered heteroaryl comprising 1-2 heteroatoms selected from O, S(O)_{q}, and N, wherein the fused phenyl or 5-6 membered heteroaryl is substituted with 0-3 R^{a};

provided that when Ar¹ comprises pyrazolyl, pyridyl, or pyrimidyl, then ring A is substituted with at least one R¹ which is other than unsubstituted alkyl;

R^{b} is independently at each occurrence H, (C₁₋₆)-alkyl, (C₃₋₆)-cycloalkyl, (C₁₋₆)alkyl-(C₃₋₆)-cycloalkyl, (3-8 membered)-heterocyclyl, (C₁₋₆)alkyl-(3-8 membered)-heterocyclyl, aryl, heteroaryl, aralkyl, or heteroarylalkyl, wherein any R^{b} other than H is substituted with 0-3 R^{a};

R² is H or (C₁₋₆)-alkyl;

n is 0 or 1;

and when n=1, Y is H,

or Y comprising —CH₂— together with Z comprising —CH₂NR^{b}—, and carbon atoms to which Y and Z are bonded can together form a 5- or 6-membered heterocyclyl ring substituted with 0-3 R^{a}, or

when n=0 or 1, Z comprises NH₂ or OH; and,

Ar² comprises aryl, heteroaryl, or is absent, wherein any aryl or heteroaryl is substituted with 0-3 R^{a},

or when Ar² is aryl or heteroaryl, Ar² together with Z comprising NR^{b} can together form an aryl- or heteroaryl-fused 5- or 6-membered heterocyclyl substituted with 0-3 R^{a};

or any salt, stereoisomer, tautomer, hydrate, solvate, or prodrug thereof.

For example, a compound of the invention comprises a compound of formula (I) wherein Z is NH₂, n=0, and Ar² comprises an aryl or heteroaryl substituted with 0-3 R^{a}, or is absent.

For example, a compound of the invention comprises a compound of formula (I) wherein Z is OH, n=0, Ar² comprises an aryl or heteroaryl substituted with 0-3 R^{a}, or is absent.

For example, a compound of the invention comprises a compound of formula (I) wherein n=0 and Ar² together with Z comprising NR² form an aryl- or heteroaryl-fused 5- or 6-membered heterocyclyl substituted with 0-3 R^{a}.

For example, a compound of the invention comprises a compound of formula (I) wherein Z is NH₂, n=1, and Ar² comprises an aryl or heteroaryl substituted with 0-3 R^{a}.

For example, a compound of the invention comprises a compound of formula (I) wherein n=1, and Y comprising —CH₂— together with Z comprising —CH₂NR^{b}—, and carbon atoms to which Y and Z are bonded can together form a 5- or 6-membered heterocyclyl ring substituted with 0-3 R^{a}.

For example, a compound of the invention comprises a compound of formula (I) wherein Ar¹ comprises

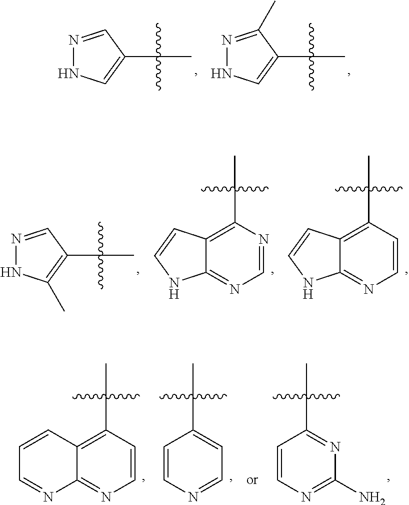

wherein a wavy line indicates a point of attachment.

For example, a compound of the invention comprises a compound of formula (I) wherein ring A comprises a phenyl, pyridyl, or pyridazinyl ring.

For example, a compound of the invention comprises a compound of formula Ia-If:

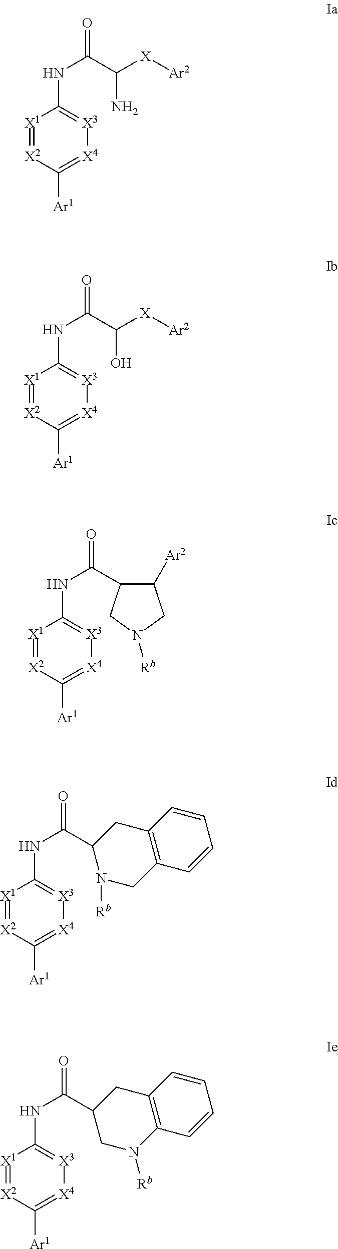

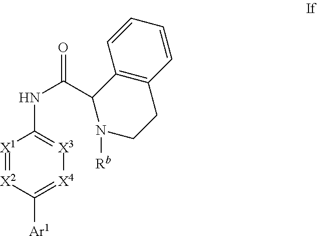

wherein X is absent or is CH₂, or any salt, stereoisomer, tautomer, hydrate, solvate, or prodrug thereof.

For example, a compound of the invention comprises a compound of formula (I) wherein each independently selected R¹ comprises chloro, fluoro, methoxy, —C(O)NR₂, —O(CH₂)ₚNR₂, —S(O)_{q}(CH₂)ₚC(O)NR₂, —S(O)_{q}(CH₂)ₚNR₂, —O(CH₂)ₚOR^{a}, N(R)(CH₂)ₚNR₂, —OCH(OH)CH₂NR₂,

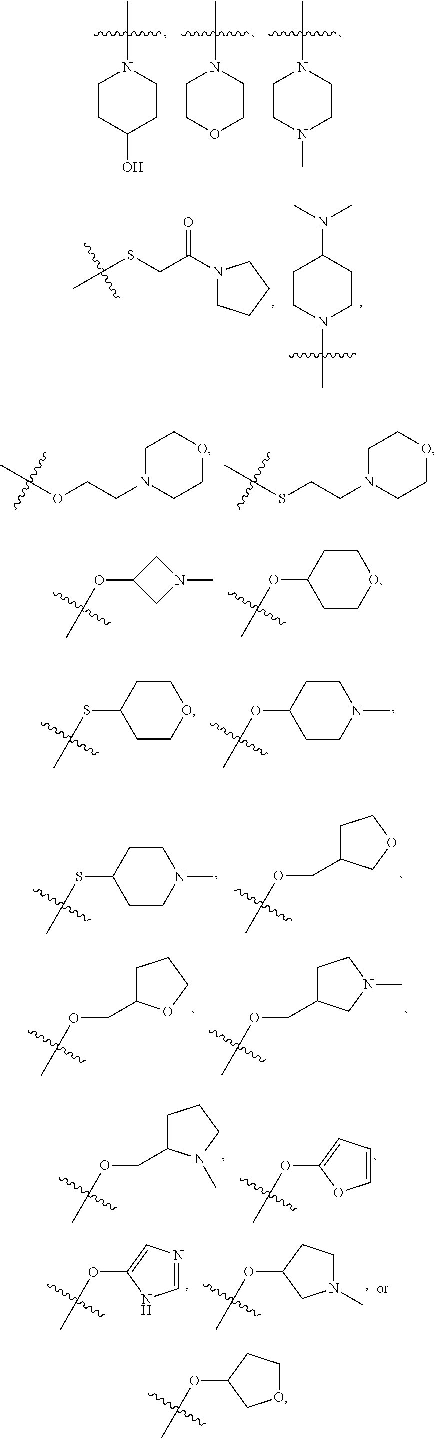

wherein a wavy line indicates a point of attachement.

In various embodiments, the invention provides a compound of any of the examples 1-141, or any tautomer, salt, stereoisomer, hydrate, solvent, or prodrug thereof.

It is to be understood that other particular and preferred embodiments of the compounds of the invention will combine the features of the particular and preferred embodiments of the invention explicitly described above. Embodiments defined by such combinations are contemplated as particular embodiments of the invention.

In other preferred embodiments the compound of formula I, or any of the embodiments thereof, is an isolated compound. In other preferred embodiments, the compound of formula I, and compositions containing the compound, including pharmaceutical compositions, are substantially free of pharmaceutically unacceptable contaminants. A pharmaceutically unacceptable contaminant is a compound which, if present in more than an insubstantial amount, would render the compound unsuitable for use as a pharmaceutical for therapeutic administration.

### Methods for Preparing Compounds of the Invention

There are provided processes for preparing compounds according to formula I, intermediates that are useful in the preparation of such compounds, and processes for preparing such intermediates. The compounds can be prepared by a variety of synthetic routes. Representative procedures are shown below in the Examples in Schemes 1-3. It will be readily apparent that the compounds can be synthesized by substitution of the appropriate starting materials, reactants, and reagents in the syntheses shown below. It will also be apparent that the selective protection and deprotection steps, as well as the order of the steps themselves, can be carried out in varying order, depending on the nature of the reactions. Precursor compounds, intermediates, and reagents are commercially available or can be prepared from commercially available starting materials. The following schemes are representative, and are in no way intended to limit the scope of the compounds in the embodiments of the present invention.

In the text, formulae and schemes that follow, unless otherwise indicated, the variables are as defined above for formula I.

The above-described reactions, unless otherwise noted, are usually conducted at a pressure of about one to about three atmospheres, preferably at ambient pressure (about one atmosphere).

The present invention further embraces isolated compounds of the invention according to formula I. The expression “isolated compound” refers to a preparation of a compound of the invention, or a mixture of compounds of the invention, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of the invention or a mixture of compounds of the invention, which contains the named compound or mixture of compounds of the invention in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

The synthetic methods described above reflect a convergent synthesis strategy. These convergent synthetic schemes allow for arrangement of the assembly steps of the backbone of the target compounds and derivatization of derivatizable functionalities to accommodate functional group sensitivity and/or to allow for functional groups or elements to be introduced either before or after the assembly of the backbone of the target compounds via the condensation and coupling reactions described.

It will be appreciated by one skilled in the art that certain aromatic substituents in the compounds of the invention, intermediates used in the processes described above, or precursors thereto, may be introduced by employing aromatic substitution reactions to introduce or replace a substituent, or by using functional group transformations to modify an existing substituent, or a combination thereof. Such reactions may be effected either prior to or immediately following the processes mentioned above, and are included as part of the process aspect of the invention. The reagents and reaction conditions for such procedures are known in the art. Specific examples of procedures which may be employed include, but are not limited to, electrophilic functionalization of an aromatic ring, for example via nitration, halogenation, or acylation; transformation of a nitro group to an amino group, for example via reduction, such as by metal/acid or catalytic hydrogenation; acylation, alkylation, or sulfonylation of an amino or hydroxyl group; replacement of an amino group by another functional group via conversion to an intermediate diazonium salt followed by nucleophilic or free radical substitution of the diazonium salt; or replacement of a halogen by another group, for example via nucleophilic or organometallically-catalyzed substitution reactions.

Additionally, in the aforesaid processes, certain functional groups which would be sensitive to the reaction conditions may be protected by protecting groups. A protecting group is a derivative of a chemical functional group which would otherwise be incompatible with the conditions required to perform a particular reaction which, after the reaction has been carried out, can be removed to re-generate the original functional group, which is thereby considered to have been “protected”, for example N-protected or O-protected, as defined above. Any chemical functionality that is a structural component of any of the reagents used to synthesize compounds of this invention may be optionally protected with a chemical protecting group if such a protecting group is useful in the synthesis of compounds of this invention. The person skilled in the art knows when protecting groups are indicated, how to select such groups, and processes that can be used for selectively introducing and selectively removing them, because methods of selecting and using protecting groups have been extensively documented in the chemical literature. Techniques for selecting, incorporating and removing chemical protecting groups may be found, for example, in Protective Groups in Organic Synthesis, Third Ed. by Theodora W. Greene, Peter G. M. Wuts (John Wiley & Sons, Inc., 1999), the entire disclosure of which is incorporated herein by reference.

In addition to use of a protecting group, sensitive functional groups may be introduced as synthetic precursors to the functional group desired in the intermediate or final product. An example of this is an aromatic nitro (—NO₂) group. The aromatic nitro group goes not undergo any of the nucleophilic reactions of an aromatic amino group. However, the nitro group can serve as the equivalent of a protected amino group because it is readily reduced to the amino group under mild conditions that are selective for the nitro group over most other functional groups.

It will be appreciated by one skilled in the art that the processes described are not the exclusive means by which compounds of the invention may be synthesized and that an extremely broad repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 1996), Comprehensive Organic Functional Group Transformations II, Ed. A. R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996).

### Treatment of Rho-Kinase Medicated Disorders Using Compounds of the Invention

According to another embodiment of the invention, a method of treating a patient suffering from Rho-kinase-mediated disorder is provided, comprising administering to the patient an effective amount of at least one compound of the invention, or any tautomer, salt, stereoisomer, hydrate, solvent, or prodrug thereof, either alone, or in combination with a pharmaceutically acceptable carrier.

The invention is also directed to the use of a compound of the invention, or a tautomer, salt, stereoisomer, hydrate, solvent, or prodrug thereof, in the preparation of a medicament for treatment of a Rho-Kinase mediated disorder

The compounds of the present invention or a tautomer, salt, stereoisomer, hydrate, solvent, or prodrug thereof can inhibit or otherwise influence an activity of any Rho kinase such as ROCK I and/or ROCK II. Therefore, the compounds of the present invention are useful for the treatment and/or prevention of a variety of Rho-kinase-mediated diseases.

Rho-kinase-mediated diseases which can be treated and/or prevented by using the compound of the present invention include, but are not limited to, hypertension, pulmonary hypertension, atherosclerosis, stroke, angina, heart failure, arterial obstruction, peripheral arterial disease, peripheral circulation disorder, vasospasm, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, multiple sclerosis, diabetes, urinary organ diseases such as overactive bladder (OAB) and benign prostatic hypertrophy (BPH), metastasis, cancer, glaucoma, ocular hypertension, retinopathy, autoimmune disease, viral infection, and myocardial protection.

Rho-kinase inhibitors of the present will also be effective for pain alleviation and cartilage protection and will therefore also be effective to treat osteoarthritis, rheumatoid arthritis. osteoporosis, and osteoarthritis.

Particular and preferred embodiments of this aspect of the invention are those wherein the compound of the invention used in the method of treatment, either alone or as part of a composition is a particular or preferred embodiment of the compound of the invention in the description of the compounds and compositions of the invention as provided herein.

The compounds according to the invention may be administered to individuals (mammals, including animals and humans) afflicted with Rho-kinase-mediated disorders as identified herein.

### Salts of Compounds According to the Invention

The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula I compounds, for example in their purification by recrystallization. All of these salts may be prepared by conventional means from the corresponding compound according to Formula I by reacting, for example, the appropriate acid or base with the compound according to Formula I.

### Isomerism and Tautomerism in Compounds of the Invention

### Tautomerism

Within the present invention it is to be understood that a compound of the formula I or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form which inhibits Rho-kinase activity, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

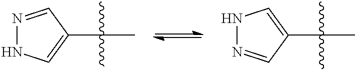

Such tautomerism can also occur with substituted pyrazoles such as 3-methyl, 5-methyl, or 3,5-dimethylpyrazoles, and the like.

### Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention which are biologically active in the treatment of Rho-kinase mediated diseases. The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

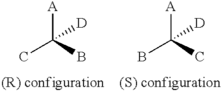

The present invention is meant to encompass diastereomers as well as their racemic and resolved, diastereomerically and enantiomerically pure forms and salts thereof.

Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (Scheme 15). It is further understood that certain structural elements, including steric bulk or substituents on the amide nitrogen, may enhance the stability of a rotamer to the extent that a compound may be isolated as, and exist indefinitely, as a single stable rotamer. The present invention therefore includes any possible stable rotamers of formula I which are biologically active in the treatment of cancer or other proliferative disease states.

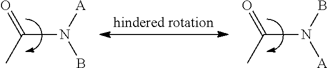

### D. Regioisomerism

The preferred compounds of the present invention have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below (Scheme 16).

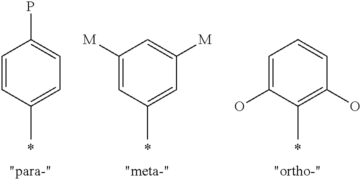

### Pharmaceutical Compositions

Another aspect of an embodiment of the invention provides compositions of the compounds of the invention, alone or in combination with another medicament. As set forth herein, compounds of the invention include stereoisomers, tautomers, solvates, prodrugs, pharmaceutically acceptable salts and mixtures thereof. Compositions containing a compound of the invention can be prepared by conventional techniques, e.g. as described in Remington: The Science and Practice of Pharmacy, 19th Ed., 1995, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tabletted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tabletting techniques can contain:

Core:

| | |
|---|---|
| Active compound (as free compound or salt thereof) | 250 mg |
| Colloidal silicon dioxide (Aerosil) ® | 1.5 mg |
| Cellulose, microcryst. (Avicel) ® | 70 mg |
| Modified cellulose gum (Ac-Di-Sol) ® | 7.5 mg |
| Magnesium stearate | Ad. |

Coating:

| | |
|---|---|
| HPMC approx. | 9 mg |
| *Mywacett 9-40 T approx. | 0.9 mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The compounds of the invention can be administered to a mammal, especially a human in need of such treatment, prevention, elimination, alleviation or amelioration of a malcondition. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician. The compounds of the invention may be administered in the form of a pharmaceutical composition, in combination with a pharmaceutically acceptable carrier. The active ingredient in such formulations may comprise from 0.1 to 99.99 weight percent.

“Pharmaceutically acceptable carrier” means any carrier, diluent or excipient which is compatible with the other ingredients of the formulation and not deleterious to the recipient. The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

The pharmaceutical compositions of the present invention may also be formulated so as to provide slow or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,354,566 discloses a controlled-release powder that contains the active ingredient. U.S. Pat. No. 5,733,566, describes the use of polymeric microparticles that release antiparasitic compositions. The controlled-release of the active ingredient may be stimulated by various inducers, for example pH, temperature, enzymes, water, or other physiological conditions or compounds.

Various mechanisms of drug release exist. For example, in one embodiment, the controlled-release component may swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

One or more compounds useful in the practice of the present inventions may be administered simultaneously, by the same or different routes, or at different times during treatment. The compounds may be administered before, along with, or after other medications. The treatment may be carried out for as long a period as necessary, either in a single, uninterrupted session, or in discrete sessions. The treating physician will know how to increase, decrease, or interrupt treatment based on patient response. The treatment schedule may be repeated as required.

### Pharmaceutical Combinations

In various embodiments, a pharmaceutical combination comprising a compound of the invention in a therapeutically effective dose and a second medicament in a therapeutically effective dose is provided. More specifically, the second medicament can comprise an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent. For example, the anti-proliferative agent can comprise an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate. For example, the anti-glaucoma agent can comprise a beta receptor-blocker, a prostaglandin, an alpha-adrenergic agonist, a parasympathomimetic (cholinergic agonist), or a carbonic anhydrase inhibitor. For example, the anti-hypertensive agent can comprise a beta receptor-blocker, a calcium channel blocker, a diueretic, an angiotensin converting enzyme (ACE) inhibitor, a renin inhibitor, or an angiotensin receptor antagonist. For example, the anti-atherosclerotic agent can comprise a 3-HMG-coA-reductase inhibitor, a statin, atorvastatin, simvastatin, niacin, or a combination drug such as vytorin. For example, the anti-multiple sclerosis agent can comprise beta-inteferon, tysabri, or glatirimar acetate. For example, the anti-angina agent can comprise a beta receptor-blocker, a calcium channel blocker, nitroglycerin, isosorbide mononitrate, nicorandil, or ranolanzine. For example, the anti-erectile dysfunction agent can comprise a phosphodiesterase-5 inhibitor. For example, the anti-stroke agent can comprise tissue plasminogen activator. For example, the anti-asthma agent can comprise a bronchodilator, an inhaled corticosteroid, a leukotrine blockers, cromolyn, nedocromil, or theophylline.

In various embodiments, a pharmaceutical combination of the invention can further comprise a suitable excipient as outlined above to provide a pharmaceutical composition comprising both medicaments.

In various embodiments, a method of treatment of a malcondition is provided comprising administering an effective amount of a compound of the invention and co-administering an effective amount of an additional medicament. The malcondition can comprise cardiovascular disease, neurogenic pain, hypertension, atherosclerosis, angina, stroke, arterial obstruction, peripheral arterial disease, peripheral circulation disorder, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the additional medicament that can be co-administered can comprise an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent. By “co-administered” is meant that the patient is provided with an effective dose of an inventive compound and with an effective dose of the second medicament during the course of treatment, such as concurrently, consecutively, intermittently, or in other regimens. The compound of the invention and the second medicament can be administered in separate dosage forms. For example, the anti-proliferative agent can comprise an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate. For example, the anti-glaucoma agent can comprise a beta receptor-blocker, a prostaglandin, an alpha-adrenergic agonist, a parasympathomimetic (cholinergic agonist), or a carbonic anhydrase inhibitor. For example, the anti-hypertensive agent can comprise a beta receptor-blocker, a calcium channel blocker, a diueretic, an angiotensin converting enzyme (ACE) inhibitor, a renin inhibitor, or an angiotensin receptor antagonist. For example, the anti-atherosclerotic agent can comprise a 3-HMG-coA-reductase inhibitor, a statin, atorvastatin, simvastatin, niacin, or a combination drug such as vytorin. For example, the anti-multiple sclerosis agent can comprise beta-inteferon, tysaberai, or glatirimar acetate. For example, the anti-angina agent can comprise a beta receptor-blocker, a calcium channel blocker, nitroglycerin, isosorbide mononitrate, nicorandil, or ranolanzine. For example, the anti-erectile dysfunction agent can comprise a phosphodiesterase-5 inhibitor. For example, the anti-stroke agent can comprise tissue plasminogen activator. For example, the anti-asthma agent can comprise a bronchodilator, an inhaled corticosteroid, a leukotrine blockers, cromolyn, nedocromil, or theophylline.

### EXAMPLES

Unless specifically stated otherwise, the experimental procedures were performed under the following conditions. All operations were carried out at room or ambient temperature—that is, at a temperature in the range of 18-25° C. Evaporation of solvent was carried out using a rotary evaporator under reduced pressure (600-4000 pascals: 4.5-30 mm. Hg) with a bath temperature of up to 60° C. The course of reactions was followed by thin layer chromatography (TLC) and reaction times are given for illustration only. The structure and purity of all final products were assured by at least one of the following techniques: TLC, mass spectrometry, nuclear magnetic resonance (NMR) spectrometry or HPLC analysis.

When given, yields are for illustration only. When given, NMR data is in the form of delta (δ) values for major diagnostic protons, given in parts per million (ppm) relative to tetramethylsilane (TMS) as internal standard, determined at 400 MHz using the indicated solvent. Conventional abbreviations used for signal shape are: s. singlet; d. doublet; t. triplet; m. multiplet; br. broad; etc. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### General Procedures:

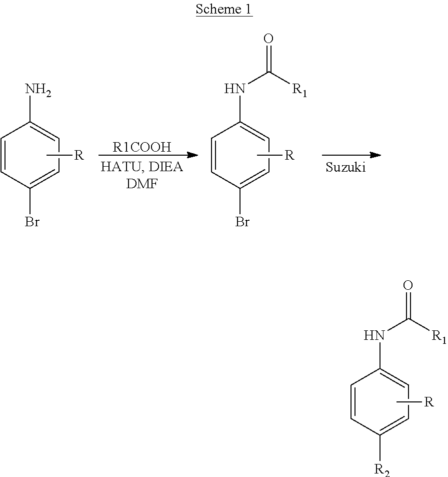

A substituted or not substituted 4-bromoaniline (1 equiv) was added to a stirred solution of an acid R₁COOH (1.2 equiv), HATU (1.3 equiv), and DIEA (3 equiv) in DMF at 23° C. After the amide coupling was finished (monitored by TLC or LC-MS or LC), the DMF was removed under reduced pressure. The residue was suspended in EtOAc, washed by brine, saturated NaHCO₃ (2×), brine (2×), 1N HCl (2×), and brine (2×) again, dried over Na₂SO₄. The solvents were then evaporated in a Rotovapor to give the crude amide, which was then used directly in the next step without further purification.

Standard Suzuki coupling conditions were utilized to prepare the final product. Thus, Pd[P(Ph)₃]₄ (15%) was added to a degassed (with Argon) solution of the bromophenyl amide (1 equiv) obtained above, a boronic acid or ester (1.5 equiv), K₂CO₃ (4 equiv) in dioxane/H₂O (4:1 by volume). The resulting suspension was sealed in Microwave reactor (from Biotage). This solution was then either subjected to microwave conditions (90° C., 2 h) or thermal conditions (95° C., 10 h) to do the Suzuki couplings. After the coupling was complete (monitored by LC-MS), the solvents were removed via a Rotovapor, and the resulting residue was directly subjected to preparative HPLC to give the final product as a TFA salt (after drying).

### Example 1

### Synthesis of 2-amino-N-(3-chloro-4-(1H-pyrazol-4-yl)phenyl)-2-(4-chlorophenyl)acetamide

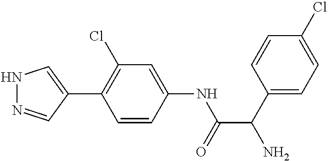

Procedures in Scheme 1 were used to prepare this titled compound. Preparative HPLC gave 43 mg of the title compound (31%) from 185 mg starting material (bromide). LC-MS: single peak at 254 nm, MH⁺ calcd. for C₁₇H₁₄Cl₂N₄O: 361, obtained: 361. ¹H-NMR (DMSO-d₆, 400 MHz), δ 10.71 (s, 1H), 8.69 (m, 3H), 7.93 (s, 2H), 7.76 (d, J=2.2 Hz, 1H), 7.54 (s, 1H), 7.52 (s, 4H), 7.38 (dd, J=8.6, 2.2 Hz, 1H), 5.06 (m, 1H).

### Example 2

### Synthesis of N-(4-(1H-pyrrolo[2,3-b]pyridin-4-yl)phenyl)-2-amino-2-(4-chlorophenyl)acetamide

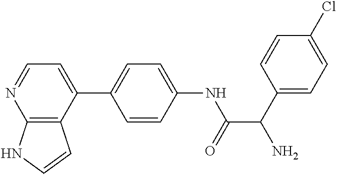

Procedures in Scheme 1 were used to prepare this titled compound. ¹H NMR (DMSO, 400 MHz) δ 5.10-5.11 (m, 1H), 6.56-6.58 (m, 1H), 7.11-7.13 (d, J=5.2 Hz, 1H), 7.48-7.57 (complex, 5H), 7.67-7.74 (m, 4H), 8.20-8.22 (d, J=5.2 Hz, 1H), 8.72 (br s, 3H), 10.7 (s, 1H), 11.8 (s, 1H); LC-MS: C₂₁H₁₈ClN₄O (M⁺+1) 377.08.

### Example 3

### Synthesis of N-(4-(7H-pyrrolo[2,3-d]pyrimidin-4-yl)phenyl)-2-amino-2-(4-chlorophenyl)acetamide

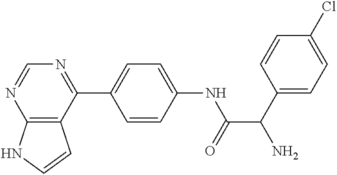

Procedures in Scheme 1 were used to prepare this titled compound. ¹H NMR (DMSO, 400 MHz) δ 5.11-5.12 (m, 1H), 6.86-6.88 (m, 1H), 7.52-7.63 (complex, 5H), 7.72-7.74 (m, 2H), 8.13-8.15 (m, 2H), 8.74-8.76 (complex, 4H), 10.83 (s, 1H), 12.29 (s, 1H); LC-MS: C₂₀H₁₇ClN₅O (M⁺+1) 378.15.

### Example 4

### Synthesis of N-(4-(1,8-naphthyridin-4-yl)phenyl)-2-amino-2-(4-chlorophenyl)acetamide

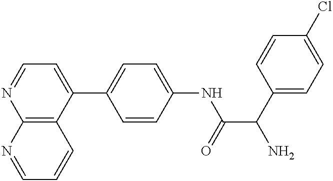

Procedures in Scheme 1 were used to prepare this titled compound. ¹H NMR (DMSO, 400 MHz) δ 5.15 (m, 1H), 7.52-7.62 (complex, 8H), 7.73-7.75 (m, 2H), 8.30-8.33 (m, 2H), 8.77 (br s, 3H), 9.07-9.09 (m, 2H), 10.87 (s, 1H); LC-MS: C₂₂H₁₈ClN₄O (M⁺+1) 389.04.

### Example 5

### Synthesis of N-(4-(1H-pyrrolo[2,3-b]pyridin-4-yl)phenyl)-2-amino-2-(2,4-difluorophenyl)acetamide

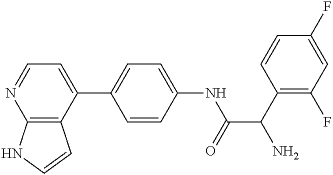

Procedures in Scheme 1 were used to prepare this titled compound. ¹H NMR (DMSO, 400 MHz) δ 5.29 (m, 1H), 6.57-6.58 (m, 1H), 7.12-7.13 (m, 1H), 7.17-7.22 (m, 1H), 7.39-7.44 (m, 1H), 7.48-7.56 (m, 2H), 7.68-7.75 (m, 4H), 8.21-8.22 (m, 1H), 8.70 (br s, 3H), 10.66 (s, 1H), 11.81 (s, 1H); LC-MS: C₂₁H₁₇F₂N₄O (M⁺+1) 379.15.

### Example 6

### Synthesis of 2-amino-2-(4-chlorophenyl)-N-(2-fluoro-5-methoxy-4-(1H-pyrazol-4-yl)phenyl)acetamide

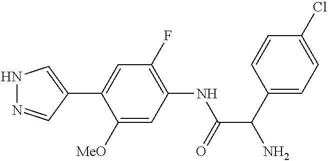

Procedures in Scheme 1 were used to prepare this titled compound. Single peak in analytical HPLC at 254 nm; LC-MS: C₁₈H₁₆ClFN₄O₂(M⁺+1) 375.

### Example 7

### Synthesis of 2-amino-2-(4-bromophenyl)-N-(2-fluoro-5-methoxy-4-(1H-pyrazol-4-yl)phenyl)acetamide

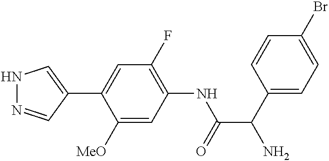

Procedures in Scheme 1 were used to prepare this titled compound. Single peak in analytical HPLC at 254 nm, LC-MS: C₁₈H₁₆FBr₄O₂(M⁺+1) 419.

### Example 8

### Synthesis of (R)-2-amino-N-(2-fluoro-5-methoxy-4-(1H-pyrazol-4-yl)phenyl)-2-(4-fluorophenyl)acetamide

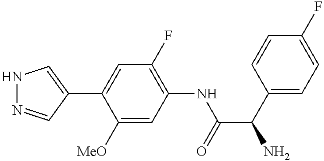

Procedures in Scheme 1 were used to prepare this titled compound. Single peak in analytical HPLC at 254 nm; LC-MS: C₁₈H₁₆F₂N₄O₂(M⁺+1) 359.

### Example 9

### Synthesis of (R)-2-amino-2-(3-chlorophenyl)-N-(2-fluoro-5-methoxy-4-(1H-pyrazol-4-yl)phenyl)acetamide

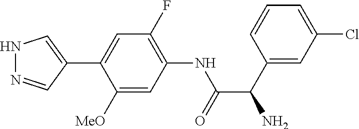

Procedures in Scheme 1 were used to prepare this titled compound. Single peak in analytical HPLC at 254 nm, LC-MS: C₁₈H₁₆FClN₄O₂(M⁺+1) 375.

### Example 10

### Synthesis of 2-amino-N-(2-fluoro-4-(1H-pyrazol-4-yl)phenyl)-2-(pyridin-3-yl)acetamide

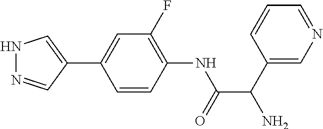

Procedures in Scheme 1 were used to prepare this titled compound. ¹H NMR (DMSO, 400 MHz) δ 5.35 (s, 1H), 7.44 (dd, J=2.8, 8.4 Hz, 1H), 7.55 (m, 2H), 7.70 (t, J=8.4 Hz, 1H), 7.99 (m, 1H), 8.09 (s, 2H), 8.67 (dd, J=1.2, 4.8 Hz, 1H), 8.80 (m, 1H), 8.83 (s, 2H), 10.47 (s, 1H); Single peak in analytical HPLC at 254 nm, LC-MS: C₁₆H₁₄FN₅O (M⁺+1) 312.

### Example 11

### Synthesis of N-(2-fluoro-4-(1H-pyrazol-4-yl)phenyl)-6-methoxy-1,2,3,4-tetrahydroisoquinoline-1-carboxamide

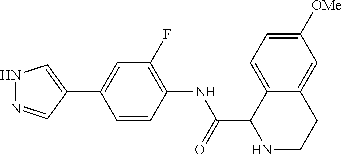

Procedures in Scheme 1 were used to prepare this titled compound. Single peak in analytical HPLC at 254 nm; LC-MS: C₂₀H₁₉FN₄O₂(M⁺+1) 367.

### Example 12

### Synthesis of 2-(2-amino-2-(4-chlorophenyl)acetamido)-N,N-dimethyl-5-(1H-pyrazol-4-yl)benzamide

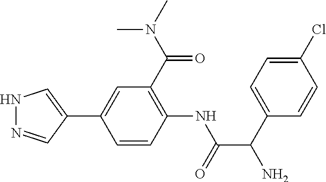

Procedures in Scheme 1 were used to prepare this titled compound. ¹H NMR (DMSO, 400 MHz) δ 2.46 (s, 3H), 2.75 (s, 3H), 5.19 (s, 1H), 7.47-7.68 (m, 7H), 8.09 (s, 2H), 8.74 (s, 1H), 10.07 (s, 1H), 12.90 (br s, 1H); Single peak in analytical HPLC at 254 nm; LC-MS: C₂₀H₂₀ClN₅O₂(M⁺+1) 398.

### Example 13

### Synthesis of (R)-2-(2-amino-3-(4-chlorophenyl)propanamido)-N,N-dimethyl-5-(1H-pyrazol-4-yl)benzamide

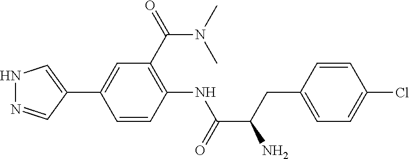

Procedures in Scheme 1 were used to prepare this titled compound. ¹H NMR (DMSO, 400 MHz) δ 2.82 (s, 3H), 2.98 (s, 3H), 2.99 (m, 1H), 3.16 (m, 1H), 4.22 (m, 1H), 7.34-7.69 (m, 7H), 8.21 (b, 4H), 10.04 (s, 1H), 12.90 (br s, 1H); Single peak in analytical HPLC at 254 nm; LC-MS: C₂₁H₂₂ClN₅O₂(M⁺+1) 412.

### Example 14

### Synthesis of (R)-1-oxo-1,2,3,4-tetrahydro-isoquinoline-3-carboxylic acid [4-(1H-pyrazol-4-yl)-phenyl]-amide

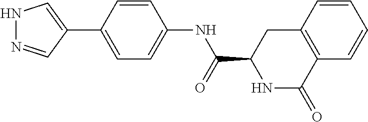

The title compound was prepared according to the procedure described in Scheme 1 except the crude product was purified by filtration and washing with water, ethanol, and ethyl acetate (16 mg, 33% over two steps). ¹H NMR (DMSO-d₆, 400 MHz) δ 10.08 (s, 1H), 7.98 (br s, 2H), 7.94 (d, J=2.8 Hz, 1H), 7.86 (d, J=7.2 Hz, 1H), 7.52 (s, 4H), 7.43 (t, J=6.4 Hz, 1H), 7.34 (t, J=7.2 Hz, 1H), 7.29 (d, J=7.6 Hz, 1H), 4.37 (m, 1H), 3.40 (dd, J=16.8, 6.4 Hz, 1H), 3.18 (dd, J=16.0, 4.8 Hz, 1H). LC-MS: single peak at 254 nm, MH⁺ calcd. for C₁₉H₁₇N₄O₂: 333, obtained: 333.

### Example 15

### Synthesis of (R)-1-Oxo-1,2,3,4-tetrahydro-isoquinoline-3-carboxylic acid [3-methoxy-4-(1H-pyrazol-4-yl)-phenyl]-amide

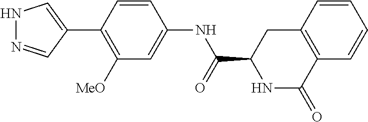

The title compound was prepared according to the procedure described in Scheme 1 (14 mg, 9% over two steps). ¹H NMR (DMSO-d₆, 400 MHz) δ 10.05 (s, 1H), 7.90 (s, 2H), 7.87 (d, J=3.2 Hz, 1H), 7.80 (dd, J=8.0, 1.6 Hz, 1H), 7.45 (d, J=8.4 Hz, 1H), 7.38 (td, J=7.6, 1.6 Hz, 1H), 7.33 (d, J=2.0 Hz, 1H), 7.27 (t, J=7.6 Hz, 1H), 7.22 (d, J=7.6 Hz, 1H), 7.05 (dd, J=8.4, 2.0 Hz, 1H), 4.31 (m, 1H), 3.74 (s, 3H), 3.40 (m, 1H), 3.12 (dd, J=16.0, 4.4 Hz, 1H). LC-MS: single peak at 254 nm, calcd. for C₁₉H₁₈N₃O₃: 336, obtained: 336.

### General Procedures:

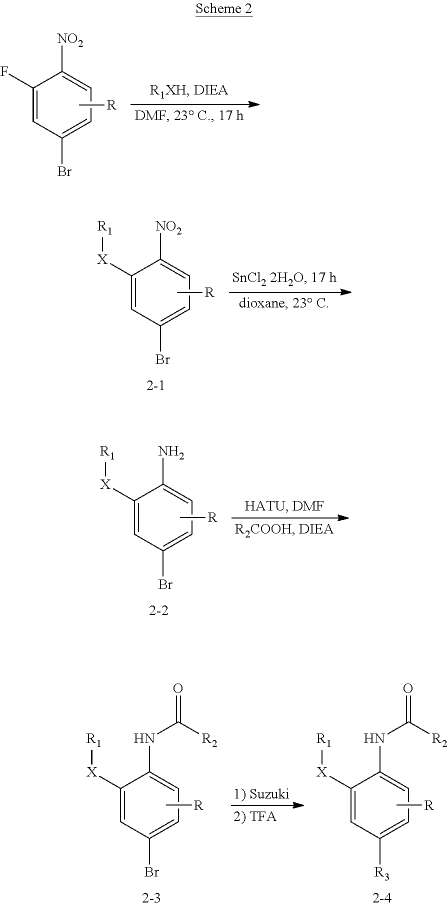

A SNAr reaction was used to prepare compound 2-1. Thus, an amine or an alcohol (1.1 equiv) was added to a solution of the 2-fluoronitrobenzene compound (1.0 equiv), and DIEA or KO^{t}BU (1.5 equiv) in DMF or THF. The suspension was gently stirred at 23° C. overnight. The solvents were then removed under reduced pressure, and the resulting residue was subjected to SnCl₂ reduction without further purification. Hydrated SnCl₂ (6 equiv) was therefore added to a solution of 2-1 in dioxane, and the suspension was stirred at 23° C. overnight. The solvent was removed under reduced pressure, and a lot excess KOH (10 equiv relative to SnCl₂) in water was added to the residue. The suspension was then extracted 4× by DCM. The organic phase was washed by brine (2×), dried over NaSO₄, and evaporated to give the crude aniline 2-2. This crude product was subjected to flash chromatography (Combi-Flash machine, a gradient MeOH in DCM was applied) to obtain the pure 2-2.

A HATU coupling method was applied to prepare the amide 2-3. Thus, HATU (1.2 equiv) was added to a solution of 2-2 (1.0 equiv), an acid RCOOH (1.1 equiv), and DIEA (3 equiv) in DMF. After gently stirring the solution at 23° C. for 2 h, the solvents were evaporated in a rotovapor. The residue was suspended in EtOAc, washed with brine (2×), saturated NaHCO₃ (2×), brine (2×), dried over Na₂SO₄, and evaporated to the crude 2-3, which was used directly in the next step without further purification. Thus, Pd[(Ph)₃]₄ (15%) was added to a degassed (using argon) solution of 2-3 (1 equiv), a boronic acid or ester (2 equiv), and K₂CO₃ (5 equiv) in dioxane/H₂O (4:1 by volume). After the solution was sealed in a high-pressure reactor, the suspension was stirred at 100° C. for 10 h, at which time the Suzuki coupling was complete based on LC-MS analysis. After removing the solvents by evaporation, the resulting residue was subjected to preparative HPLC to give the protected product 2-4. The solvents were then removed by lyophilization, and the residue was treated by 30% TFA in DCM for 30 min to give the final free amino product 2-4.

### Example 16

### Synthesis of (R)-2-amino-N-(5-fluoro-2-methoxy-4-(1H-pyrazol-4-yl)phenyl)-2-(4-fluorophenyl)acetamide

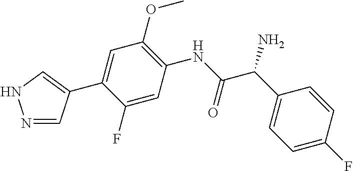

Procedures in Scheme 2 were used to prepare this titled compound. Single peak in analytical HPLC at 254 nm, LC-MS: single peak at 254 nm, MH⁺ calcd. for C₁₈H₁₆F₂N₄O₂: 359, obtained: 359.

### Example 17

### Synthesis of 2-amino-2-(4-chlorophenyl)-N-(5-fluoro-2-methoxy-4-(1H-pyrazol-4-yl)phenyl)acetamide

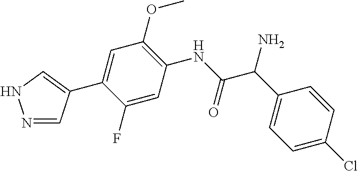

Procedures in Scheme 2 were used to prepare this titled compound. Single peak in analytical HPLC at 254 nm, LC-MS: single peak at 254 nm, MH⁺ calcd. for C₁₈H₁₆ClFN₄O₂: 375, obtained: 375.

### Example 18

### Synthesis of 2-amino-N-(5-fluoro-2-methoxy-4-(1H-pyrazol-4-yl)phenyl)-2-(3-fluorophenyl)acetamide

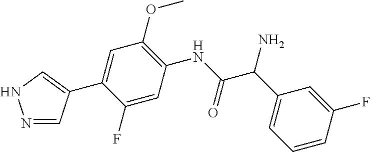

Procedures in Scheme 2 were used to prepare this titled compound. ¹H-NMR (DMSO-d₆, 400 MHz), δ: 10.10 (s, 1H), 8.82 (s, 3H), 8.07 (bs, 2H), 7.86 (d, J=12.8 Hz, 1H), 7.55 (m, 2H), 7.33 (m, 3H), 5.46 (s, 1H), 3.88 (s, 3H). LC-MS: single peak at 254 nm, MH⁺ calcd. for C₁₈H₁₆F₂N₄O₂: 359, obtained: 359.

### Example 19

### Synthesis of 2-amino-2-(4-chlorophenyl)-N-(2-(2-(dimethylamino)ethoxy)-5-fluoro-4-(1H-pyrazol-4-yl)phenyl)acetamide

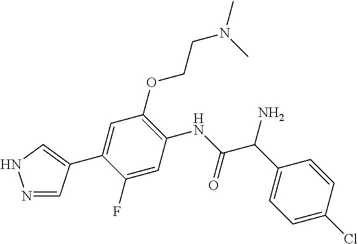

Procedures in Scheme 2 were used to prepare this titled compound. ¹H-NMR (DMSO-d₆, 400 MHz), δ: 10.00 (b, 1H), 9.87 (s, 1H), 8.9 (b, 3H), 8.06 (bs, 2H), 7.88 (d, J=12.4 Hz, 1H), 7.60 (m, 4H), 7.40 (d, J=J=2.8 Hz, 1H), 5.45 (s, 1H), 4.44 (m, 1H), 4.36 (m, 1H), 3.44 (m, 1H), 3.41 (m, 1H), 2.30 (s, 6H). LC-MS: single peak at 254 nm, MH⁺ calcd. for C₂₁H₂₃ClFN₅O₂: 432, obtained: 432.

### Example 20

### Synthesis of 2-amino-N-(2-(2-(dimethylamino)ethoxy)-5-fluoro-4-(1H-pyrazol-4-yl)phenyl)-2-(3-fluorophenyl)acetamide

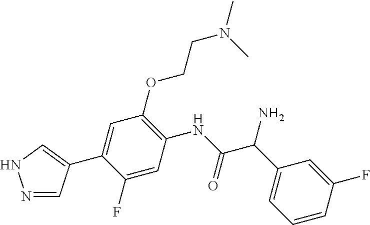

Procedures in Scheme 2 were used to prepare this titled compound. LC-MS: single peak at 254 nm, MH⁺ calcd. for C₂₁H₂₃F₂N₅O₂: 416, obtained: 416.

### Example 21

### Synthesis of (R)-2-amino-N-(2-(2-(dimethylamino)ethoxy)-5-fluoro-4-(1H-pyrazol-4-yl)phenyl)-3-(4-(trifluoromethyl)phenyl)propanamide

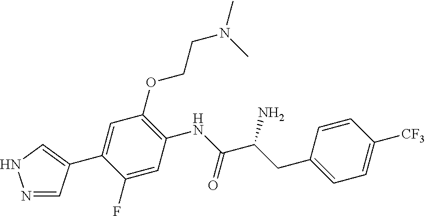

Procedures in Scheme 2 were used to prepare this titled compound. LC-MS: single peak at 254 nm, MH⁺ calcd. for C₂₃H₂₅F₄N₅O₂: 478, obtained: 478.

### Example 22

### Synthesis of (R)-2-amino-3-(4-chlorophenyl)-N-(2-(2-(dimethylamino)ethoxy)-5-fluoro-4-(1H-pyrazol-4-yl)phenyl)propanamide

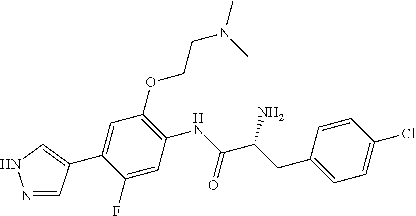

Procedures in Scheme 2 were used to prepare this titled compound. LC-MS: single peak at 254 nm, MH⁺ calcd. for C₂₂H₂₅FClN₅O₂: 446, obtained: 446.

### Example 23

### Synthesis of N-(2-fluoro-5-methoxy-4-(1H-pyrazol-4-yl)phenyl)chroman-3-carboxamide

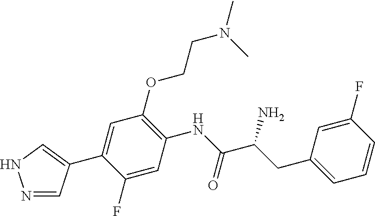

Procedures in Scheme 2 were used to prepare this titled compound. LC-MS: single peak at 254 nm, MH⁺ calcd. for C₂₂H₂₅F₂N₅O₂: 430, obtained: 430.

### Example 24

### Synthesis of 2-amino-N-(2-(3-(dimethylamino)propoxy)-5-fluoro-4-(1H-pyrazol-4-yl)phenyl)-2-(3-fluorophenyl)acetamide

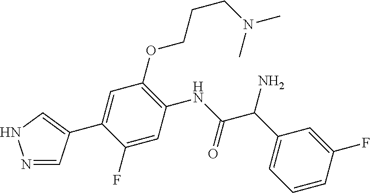

Procedures in Scheme 2 were used to prepare this titled compound. LC-MS: single peak at 254 nm, MH⁺ calcd. for C₂₂H₂₅F₂N₅O₂: 430, obtained: 430.

### Example 25

### Synthesis of 2-amino-2-(4-chlorophenyl)-N-(2-(3-(dimethylamino)propoxy)-5-fluoro-4-(1H-pyrazol-4-yl)phenyl)acetamide

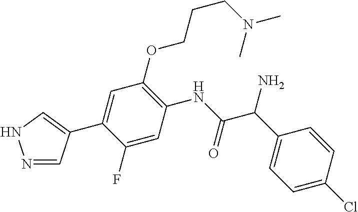

Procedures in Scheme 2 were used to prepare this titled compound. LC-MS: single peak at 254 nm, MH⁺ calcd. for C₂₂H₂₄FClN₅O₂: 446, obtained: 446.

### Example 26

### Synthesis of (R)-2-amino-N-(2-(2-(dimethylamino)ethoxy)-5-fluoro-4-(1H-pyrazol-4-yl)phenyl)-2-(4-fluorophenyl)acetamide

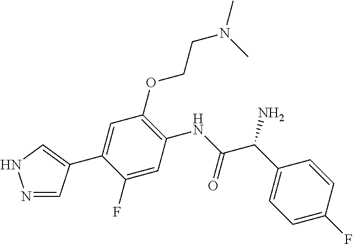

Procedures in Scheme 2 were used to prepare this titled compound. ¹H-NMR (DMSO-d₆, 400 MHz), δ: 13.00 (b, 1H), 10.60 (b, 1H), 10.27 (s, 1H), 8.84 (s, 3H), 8.06 (bs, 2H), 7.94 (d, J=12.8 Hz, 1H), 7.78 (m, 2H), 7.37 (m, 3H), 5.91 (s, 1H), 4.45 (m, 1H), 4.34 (m, 1H), 3.46 (m, 2H), 2.75 (s, 6H). LC-MS: single peak at 254 nm, MH⁺ calcd. for C₂₁H₂₂F₂N₅O₂: 416, obtained: 416.

### Example 27

### Synthesis of N-(2-(2-(dimethylamino)ethoxy)-5-fluoro-4-(1H-pyrazol-4-yl)phenyl)-6-methoxy-1,2,3,4-tetrahydroisoquinoline-1-carboxamide

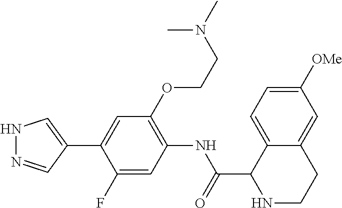

Procedures in Scheme 2 were used to prepare this titled compound. LC-MS: single peak at 254 nm, MH⁺ calcd. for C₂₄H₂₈FN₅O₃: 454, obtained: 454.

### Example 28

### Synthesis of N-(2-(2-(dimethylamino)ethoxy)-5-fluoro-4-(1H-pyrazol-4-yl)phenyl)-6,7-dimethoxy-1,2,3,4-tetrahydroisoquinoline-1-carboxamide

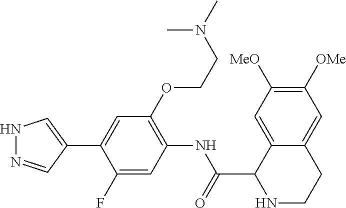

Procedures in Scheme 4 were used to prepare this titled compound. LC-MS: single peak at 254 nm, MH⁺ calcd. for C₂₅H₃₀FN₅O₄: 484, obtained: 484.

### Example 29

### Synthesis of (R)—N-(2-(2-(dimethylamino)ethoxy)-5-fluoro-4-(1H-pyrazol-4-yl)phenyl)-1,2,3,4-tetrahydroisoquinoline-3-carboxamide

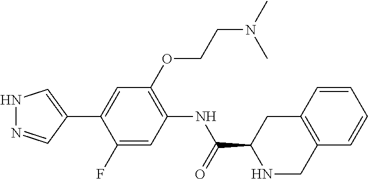

Procedures in Scheme 2 were used to prepare this titled compound. ¹H-NMR (DMSO-d₆, 400 MHz), δ: 10.10 (b, 1H), 10.00 (s, 1H), 9.80 (b, 1H), 9.62 (b, 1H), 8.10 (s, 2H), 7.87 (d, J=12.4 Hz, 1H), 7.48 (d, J=6.8 Hz, 1H), 7.28 (m, 4H), 4.49 (m, 3H), 4.41 (m, 2H), 3.60 (s, 2H), 3.45 (dd, J=4.4 Hz, J=16.8 Hz, 1H), 3.14 (dd, J=12.4 Hz, J=16.8 Hz, 1H), 2.93 (s, 6H). LC-MS: single peak at 254 nm, MH⁺ calcd. for C₂₃H₂₆FN₅O₂: 414, obtained: 414.

### Example 30

### Synthesis of (S)—N-(2-(2-(dimethylamino)ethoxy)-5-fluoro-4-(1H-pyrazol-4-yl)phenyl)-1,2,3,4-tetrahydroisoquinoline-3-carboxamide

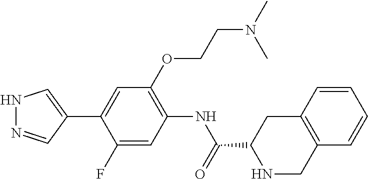

Procedures in Scheme 2 were used to prepare this titled compound. ¹H-NMR (DMSO-d₆, 400 MHz), δ: 10.15 (b, 1H), 10.02 (s, 1H), 9.82 (b, 1H), 9.62 (b, 1H), 8.10 (s, 2H), 7.88 (d, J=12.4 Hz, 1H), 7.48 (d, J=7.2 Hz, 1H), 7.28 (m, 4H), 4.49 (m, 3H), 4.41 (m, 2H), 3.60 (s, 2H), 3.42 (dd, J=4.4 Hz, J=16.8 Hz, 1H), 3.15 (dd, J=12.4 Hz, J=16.8 Hz, 1H), 2.93 (s, 6H). LC-MS: single peak at 254 nm, MH⁺ calcd. for C₂₃H₂₆N₅O₂: 414, obtained: 414.

### Example 31

### Synthesis of 2-amino-N-(2-(2-(dimethylamino)ethoxy)-5-fluoro-4-(1H-pyrazol-4-yl)phenyl)-2-(4-methoxyphenyl)acetamide

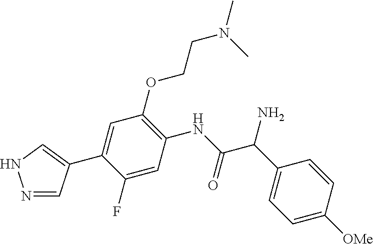

Procedures in Scheme 2 were used to prepare this titled compound. ¹H-NMR (DMSO-d₆, 400 MHz), δ 10.05 (b, 1H), 9.78 (s, 1H), 8.75 (b, 3H), 8.06 (s, 2H), 7.90 (d, J=12.4 Hz, 1H), 7.52 (d, J=8.8 Hz, 2H), 7.39 (d, J=7.2 Hz, 1H), 7.05 (d, J=6.8 Hz, 2H), 5.41 (s, 1H), 4.42 (m, 1H), 4.35 (m, 1H), 3.78 (s, 3H), 3.43 (m, 2H), 2.79 (s, 3H), 2.76 (s, 3H). LC-MS: single peak at 254 nm, MH⁺ calcd. for C₂₂H₂₆FN₅O₃: 428, obtained: 428.

### Example 32

### Synthesis of (3S,4R)—N-(2-(2-(dimethylamino)ethoxy)-5-fluoro-4-(1H-pyrazol-4-yl)phenyl)-4-phenylpyrrolidine-3-carboxamide

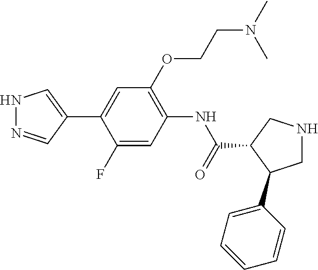

Procedures in Scheme 2 were used to prepare this titled compound. ¹H-NMR (DMSO-d₆, 400 MHz), δ 10.06 (b, 1H), 9.51 (b, 1H), 9.40 (s, 1H), 9.32 (b, 1H), 8.04 (s, 2H), 7.86 (d, J=12.8 Hz, 1H), 7.34 (m, 6H), 4.38 (m, 1H), 4.27 (m, 1H), 3.68 (m, 4H), 3.46 (m, 4H), 2.82 (s, 3H), 2.80 (s, 3H). LC-MS: single peak at 254 nm, MH⁺ calcd. for C₂₄H₂₈FN₅O₂: 438, obtained: 438.

### Example 33

### Synthesis of 2-amino-N-(5-fluoro-2-(4-hydroxypiperidin-1-yl)-4-(1H-pyrazol-4-yl)phenyl)-2-(4-methoxyphenyl)acetamide

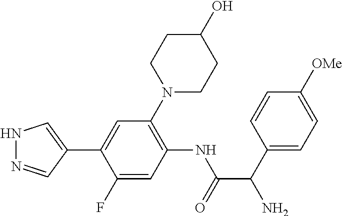

Procedures in Scheme 2 were used to prepare this titled compound. LC-MS: single peak at 254 nm, MH⁺ calcd. for C₂₃H₂₆FN₅O₃: 440, obtained: 440.

### Example 34

### Synthesis of (R)-2-amino-N-(5-fluoro-2-(4-hydroxypiperidin-1-yl)-4-(1H-pyrazol-4-yl)phenyl)-2-(4-fluorophenyl)acetamide

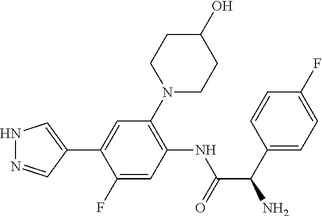

Procedures in Scheme 2 were used to prepare this titled compound. ¹H-NMR (DMSO-d₆, 400 MHz), δ: 9.32 (b, 1H), 9.24 (b, 1H), 9.23 (s, 1H), 9.00 (b, 1H), 8.04 (bs, 2H), 7.94 (d, J=13.2 Hz, 1H), 7.62 (s, 1H), 7.45 (d, J=8.0 Hz, 1H), 7.37 (m, 2H), 7.28 (m, 1H), 5.50 (s, 1H), 4.82 (s, 1H), 4.00-3.30 (m, 5H), 2.60 (m, 4H). LC-MS: single peak at 254 nm, MH⁺ calcd. for C₂₂H₂₃F₂N₅O₂: 428, obtained: 428.

### Example 35

### Synthesis of 2-amino-2-(4-chlorophenyl)-N-(5-fluoro-2-(4-hydroxypiperidin-1-yl)-4-(1H-pyrazol-4-yl)phenyl)acetamide

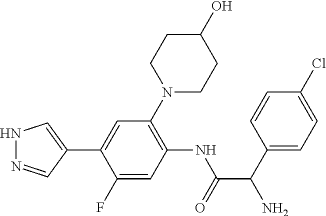

Procedures in Scheme 2 were used to prepare this titled compound. LC-MS: single peak at 254 nm, MH⁺ calcd. for C₂₂H₂₃ClFN₅O₂: 444, obtained: 444.

### Example 36

### Synthesis of (R)-2-amino-3-(4-chlorophenyl)-N-(5-fluoro-2-(4-hydroxypiperidin-1-yl)-4-(1H-pyrazol-4-yl)phenyl)propanamide

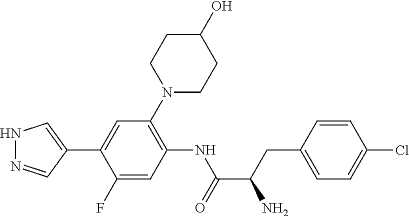

Procedures in Scheme 2 were used to prepare this titled compound. LC-MS: single peak at 254 nm, MH⁺ calcd. for C₂₃H₂₅ClFN₅O₂: 458, obtained: 458.

### Example 37

### Synthesis of (R)-2-amino-N-(5-fluoro-2-(4-hydroxypiperidin-1-yl)-4-(1H-pyrazol-4-yl)phenyl)-3-(3-fluorophenyl)propanamide

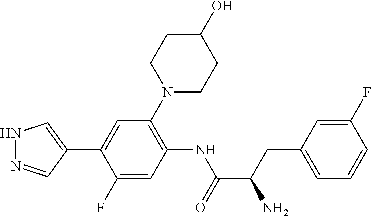

Procedures in Scheme 2 were used to prepare this titled compound. ¹H-NMR (DMSO-d₆, 400 MHz), δ: 13.00 (b, 1H), 9.34 (s, 1H), 8.30 (b, 4H), 8.04 (bs, 2H), 7.86 (d, J=12.8 Hz, 1H), 7.47 (d, J=8.0 Hz, 1H), 7.37 (m, 2H), 7.13 (m, 2H), 4.73 (b, 1H), 4.57 (m, 2H), 4.25 (m, 1H), 3.70-2.50 (m, 5H), 1.78 (m, 2H), 1.61 (m, 2H). LC-MS: single peak at 254 nm, MH⁺ calcd. for C₂₃H₂₅F₂N₅O₂: 442, obtained: 442.

### Example 38

### Synthesis of (R)—N-(5-fluoro-2-(4-hydroxypiperidin-1-yl)-4-(1H-pyrazol-4-yl)phenyl)-1,2,3,4-tetrahydroisoquinoline-3-carboxamide

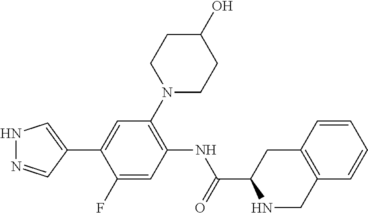

Procedures in Scheme 2 were used to prepare this titled compound. LC-MS: single peak at 254 nm, MH⁺ calcd. for C₂₄H₂₆FN₅O₂: 436, obtained: 436.

### Example 39

### Synthesis of (R)-2-amino-N-(5-fluoro-2-(4-hydroxypiperidin-1-O-4-(1H-pyrazol-4-yl)phenyl)-2-(4-fluorophenyl)acetamide

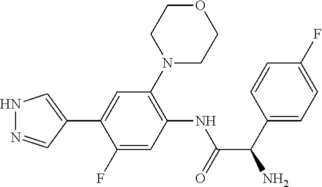

Procedures in Scheme 2 were used to prepare this titled compound. ¹H-NMR (DMSO-d₆, 400 MHz), δ: 13.00 (b, 1H), 9.41 (s, 1H), 8.80 (s, 3H), 8.06 (bs, 2H), 7.85 (d, J=12.4 Hz, 1H), 7.49 (d, J=8.0 Hz, 1H), 7.34 (m, 2H), 5.51 (s, 1H), 3.50 (m, 4H), 2.66 (m, 4H). LC-MS: single peak at 254 nm, MH⁺ calcd. for C₂₁H₂₁F₂N₅O₂: 414, obtained: 414.

### Example 40

### Synthesis of 2-amino-N-(5-fluoro-2-morpholino-4-(1H-pyrazol-4-yl)phenyl)-2-(4-methoxyphenyl)acetamide

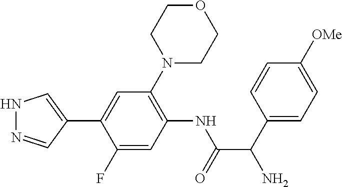

Procedures in Scheme 2 were used to prepare this titled compound. LC-MS: single peak at 254 nm, MH⁺ calcd. for C₂₂H₂₄FN₅O₃: 426, obtained: 426.

### Example 41

### Synthesis of 2-amino-2-(4-chlorophenyl)-N-(5-fluoro-2-morpholino-4-(1H-pyrazol-4-yl)phenyl)acetamide

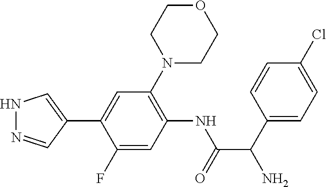

Procedures in Scheme 2 were used to prepare this titled compound. LC-MS: single peak at 254 nm, MH⁺ calcd. for C₂₁H₂₁ClFN₅O₂: 430, obtained: 430.

### Example 42

### Synthesis of (3S,4R)—N-(5-fluoro-2-morpholino-4-(1H-pyrazol-4-yl)phenyl)-4-phenylpyrrolidine-3-carboxamide

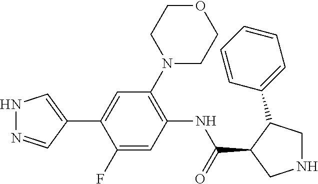

Procedures in Scheme 2 were used to prepare this titled compound. ¹H-NMR (DMSO-d₆, 400 MHz), δ: 13.00 (b, 1H), 9.35 (s, 1H), 8.78 (b, 3H), 8.03 (bs, 2H), 7.89 (d, J=12.8 Hz, 1H), 7.66 (m, 2H), 7.54 (m, 1H), 7.45 (d, J=8.0 Hz, 1H), 7.32 (m, 2H), 5.06 (s, 1H), 4.66 (s, 1H), 3.60-3.10 (m, 4H), 2.88 (m, 1H), 2.62 (m, 2H), 2.36 (m, 1H), 1.66 (m, 1H), 1.45 (m, 2H), 1.27 (m, 1H). LC-MS: single peak at 254 nm, MH⁺ calcd. for C₂₄H₂₆FN₅O₂: 436, obtained: 436.

### Example 43

### (+)-trans-N-(2-methoxy-4-(1H-pyrazol-4-yl)phenyl)-4-phenylpyrrolidine-3-carboxamide

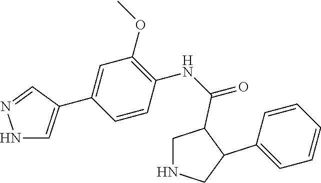

This product was obtained using 4-bromo-2-methoxyaniline and trans-N—BOC-4-phenylpyrrolidine-3-carboxylic acid in the amide coupling step and 4-(4,4,5,5-tetramethyl-1,3,2-dioxaborolan-2-yl)-1H-pyrazole as the boronic ester coupling partner in the Suzuki heteroarylation step, followed by BOC deprotection as described in Scheme 1. Data for this compound: MS: MH⁺ calcd. for C₂₁H₂₃N₄O₂⁺: 363.2, obtained: 363.2.

### Example 44

### (+)-trans-N-(5-methoxy-6-(1H-pyrazol-4-yl)pyridin-3-yl)-4-phenylpyrrolidine-3-carboxamide

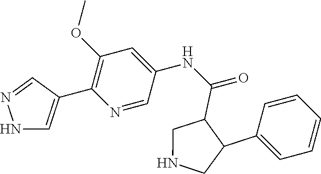

This product was obtained using 6-chloro-5-methoxypyridine-3-amine (US 2005288299) and trans-N—BOC-4-phenylpyrrolidine-3-carboxylic acid in the amide coupling step and 4-(4,4,5,5-tetramethyl-1,3,2-dioxaborolan-2-yl)-1H-pyrazole as the boronic ester coupling partner in the Suzuki heteroarylation step, followed by BOC deprotection as described in Scheme 1. Data for this compound: MS: MH⁺ calcd. for C₂₀H₂₂N₅O₂⁺: 363.2, obtained: 364.2.

### Example 45

### N-(2-fluoro-4-(1H-pyrazol-4-yl)phenyl)-6-methoxy-1,2,3,4-tetrahydroisoquinoline-3-carboxamide

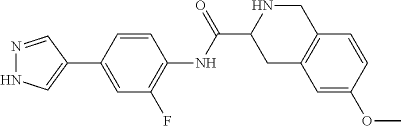

The titled compound was synthesized based on procedures in Scheme 1. LCMS (found 367.1 MH+ calculated for C₂₀H₁₉FN₄O₂: 367.2). Single peak by analytical HPLC.

### Example 46

### 6-ethoxy-N-(2-fluoro-4-(1H-pyrazol-4-yl)phenyl)-1,2,3,4-tetrahydroisoquinoline-3-carboxamide

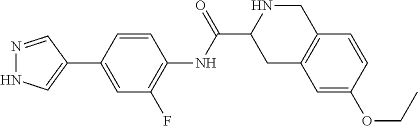

The titled compound was synthesized based on procedures in Scheme 1. LCMS (found 381 MH+ calculated for C₂₁H₂₁NFNO₂: 381). Single peak by analytical HPLC.

### Example 47

### Synthesis of (R)-1-Oxo-1,2,3,4-tetrahydro-isoquinoline-3-carboxylic acid [2-(2-dimethylamino-ethoxy)-4-(1H-pyrazol-4-O-phenyl]-amide

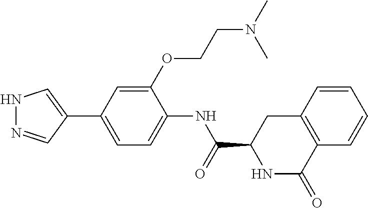

### Step A. (R)-1-Oxo-1,2,3,4-tetrahydro-isoquinoline-3-carboxylic acid

D-phenylalanine methyl ester (0.600 g, 2.78 mmol) was suspended in methylene chloride (15 mL) and a saturated solution of NaHCO₃ (15 mL) at 0° C. and stirred vigorously for 20 min. A solution of triphosgene (0.275 g, 0.923 mmol) in methylene chloride (3 mL) was added and the reaction mixture was stirred at 0° C. for 45 min. The layers were separated and the aqueous layer was further extracted twice with methylene chloride (5 mL). The combined layers were dried over Na₂SO₄, filtered, treated with AlCl₃ (0.850 g, 6.39 mmol), and refluxed overnight. After cooling to room temperature, the reaction mixture was slowly treated with water (20 mL). The layers were separated, and the organic layer was dried over Na₂SO₄, filtered, and concentrated to dryness. The residue was purified by flash column chromatography (10-55% ethyl acetate in hexanes) to afford (R)-1-oxo-1,2,3,4-tetrahydro-isoquinoline-3-carboxylic acid methyl ester as a colorless solid (0.440 g, 1.20 mmol, 77%).

The methyl ester (0.256 g, 1.25 mmol) was dissolved in methanol (1 mL) and THF (1 mL). To this solution was added LiOH (32 mg, 1.33 mmol) in water (1 mL) and the reaction was stirred overnight at room temperature. The solution was concentrated, acidified with an aqueous 1 N HCl solution to pH 1, and extracted with ethyl acetate (3×5 mL). The combined organic layers were washed with brine, dried over Na₂SO₄, filtered, and concentrated to dryness to give the title compound (0.242 g, quantitative yield). ¹H NMR (DMSO-d₆, 400 MHz) δ 12.85 (br s, 1H), 7.98 (br s, 1H), 7.84 (dd, J=7.6, 1.6 Hz, 1H), 7.46 (td, J=7.6, 1.6 Hz, 1H), 7.34 (t, J=7.6 Hz, 1H), 7.30 (d, J=7.6 Hz, 1H), 4.22 (dt, J=7.6, 4.0 Hz, 1H), 3.34 (m, 1H), 3.14 (dd, J=16.0, 3.6 Hz, 1H).

### Step B. (R)-1-Oxo-1,2,3,4-tetrahydro-isoquinoline-3-carboxylic acid [2-(2-dimethylamino-ethoxy)-4-(1H-pyrazol-4-yl)-phenyl]-amide

The title compound was prepared according to the procedure described in Scheme 1 (0.034 g, 43% over two steps). ¹H NMR (DMSO-d₆, 400 MHz) δ 9.55 (br s, 1H), 9.12 (s, 1H), 8.26 (d, J=4.0 Hz, 1H), 8.03 (br s, 2H), 7.88 (dd, J=8.0, 1.2 Hz, 1H), 7.82 (d, J=8.0 Hz, 1H), 7.48 (td, J=7.6, 1.6 Hz, 1H), 7.37-7.30 (m, 3H), 7.19 (dd, J=8.4, 1.6 Hz, 1H), 4.52-4.49 (m, 1H), 4.46-4.31 (m, 2H), 3.56 (m, 2H), 3.39 (dd, J=16.4, 6.4 Hz, 1H), 3.29 (dd, J=16.4, 4.4 Hz, 1H), 2.96 (d, J=4.8 Hz, 6H). LC-MS: single peak at 254 nm, MH⁺ calcd. for C₂₃H₂₆N₅O₃: 420, obtained: 420.

### Example 48

### Synthesis of (S)-1-Oxo-1,2,3,4-tetrahydro-isoquinoline-3-carboxylic acid [2-methoxy-4-(1H-pyrazol-4-yl)-phenyl]-amide

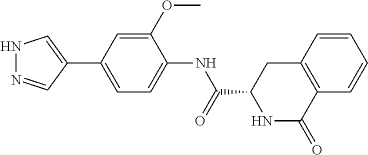

### Step A. (R)-1-Oxo-1,2,3,4-tetrahydro-isoquinoline-3-carboxylic acid

The title compound was prepared according to the procedure described in Example 47, Step A. ¹H NMR (DMSO-d₆, 400 MHz) δ 12.85 (br s, 1H), 7.98 (d, J=4.0 Hz, 1H), 7.84 (dd, J=7.6, 1.6 Hz, 1H), 7.46 (td, J=7.6, 1.6 Hz, 1H), 7.34 (t, J=7.6 Hz, 1H), 7.30 (d, J=7.6 Hz, 1H), 4.22 (dt, J=6.8, 4.0 Hz, 1H), 3.35 (m, 1H), 3.14 (dd, J=16.0, 3.6 Hz, 1H).

### Step B. (S)-1-Oxo-1,2,3,4-tetrahydro-isoquinoline-3-carboxylic acid [2-methoxy-4-(1H-pyrazol-4-yl)-phenyl]-amide

The title compound was prepared according to the procedure described in Scheme 1 (0.033 g, 26% over two steps). ¹H NMR (DMSO-d₆, 400 MHz) δ 9.21 (s, 1H), 8.13 (s, 2H), 7.85 (dd, J=8.0, 1.2 Hz, 1H), 7.82 (d, J=8.4 Hz, 1H), 7.45 (td, J=7.6, 1.2 Hz, 1H), 7.33 (t, J=7.6 Hz, 1H), 7.29 (d, J=7.6 Hz, 1H), 7.24 (d, J=2.0 Hz, 1H), 7.10 (dd, J=8.0, 1.6 Hz, 1H), 4.52 (m, 1H), 3.88 (s, 3H), 3.36 (dd, J=11.6, 6.4 Hz, 1H), 3.19 (dd, J=16.4, 4.0 Hz, 1H). LC-MS: single peak at 254 nm, MH⁺ calcd. for C₂₀H₂₁₉N₄O₃: 363, obtained: 363.

### Example 49

### Synthesis of (S)-1-Oxo-1,2,3,4-tetrahydro-isoquinoline-3-carboxylic acid [2-(2-dimethylamino-ethoxy)-4-(1H-pyrazol-4-yl)-phenyl]-amide

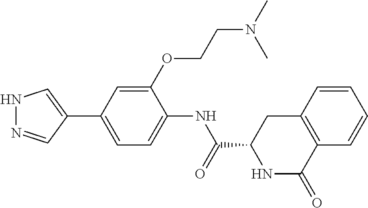

The title compound was prepared according to the procedure described in Scheme 1 (0.046 g, 27% over two steps). ¹H NMR (DMSO-d₆, 400 MHz) δ 9.55 (br s, 1H), 9.14 (s, 1H), 8.26 (d, J=4.0 Hz, 1H), 8.03 (br s, 2H), 7.88 (dd, J=7.6, 1.2 Hz, 1H), 7.82 (d, J=8.0 Hz, 1H), 7.48 (td, J=7.6, 1.2 Hz, 1H), 7.35 (t, J=7.2, 1.2 Hz, 1H), 7.31 (m, 2H), 7.19 (dd, J=8.4, 1.6 Hz, 1H), 4.53-4.51 (m, 1H), 4.46-4.39 (m, 2H), 3.56 (m, 2H), 3.39 (dd, J=16.4, 6.4 Hz, 1H), 3.29 (dd, J=16.4, 4.4 Hz, 1H), 2.96 (d, J=4.8 Hz, 6H). LC-MS: single peak at 254 nm, MH⁺ calcd. for C₂₃H₂₆N₅O₃: 420, obtained: 420.

### Example 50

### (R)—N-(2-(dimethylcarbamoyl)-4-(1H-pyrazol-4-yl)phenyl)-1,2,3,4-tetrahydroisoquinoline-3-carboxamide

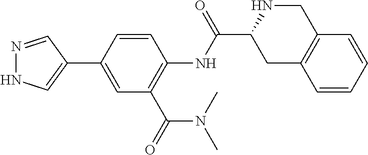

The titled compound was synthesized based on procedures in Scheme 1. LCMS (found 390 MH+ calculated for C₂₂H₂₃N₅O₂: 390. Single peak by HPLC.

### Example 51

### (R)—N-(2-(2-(dimethylamino)ethoxy)-4-(1H-pyrazol-4-yl)phenyl)-1,2,3,4-tetrahydroisoquinoline-3-carboxamide

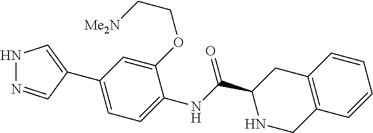

The title compound was prepared according to the procedure described in Scheme 2. LC-MS: single peak at 254 nm, MH⁺ calcd. For C₂₃H₂₇N₅O₂: 406, obtained: 406.

### Example 52

### (R)—N-(2-(2-(dimethylamino)ethoxy)-4-(3-methyl-1H-pyrazol-4-yl)phenyl)-1,2,3,4-tetrahydroisoquinoline-3-carboxamide

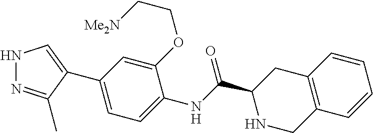

The title compound was prepared according to the procedure described in Scheme 2. LC-MS: single peak at 254 nm, MH⁺ calcd. For C₂₄H₂₉N₅O₂: 420, obtained: 420.

### Example 53

### (S)—N-(2-(2-(dimethylamino)ethoxy)-4-(1H-pyrazol-4-yl)phenyl)-1,2,3,4-tetrahydroisoquinoline-3-carboxamide

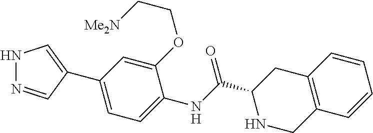

The title compound was prepared according to the procedure described in Scheme 2. LC-MS: single peak at 254 nm, MH⁺ calcd. For C₂₃H₂₇N₅O₂: 406, obtained: 406.

### Example 54

### (S)—N-(2-(2-(dimethylamino)ethoxy)-4-(3-methyl-1H-pyrazol-4-yl)phenyl)-1,2,3,4-tetrahydroisoquinoline-3-carboxamide

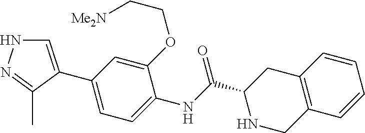

The title compound was prepared according to the procedure described in Scheme 2. LC-MS: single peak at 254 nm, MH⁺ calcd. For C₂₄H₂₉N₅O₂: 420, obtained: 420.

### Example 55

### (R)—N-(2-(3-(dimethylamino)-3-oxopropylthio)-4-(1H-pyrazol-4-yl)phenyl)-1,2,3,4-tetrahydroisoquinoline-3-carboxamide

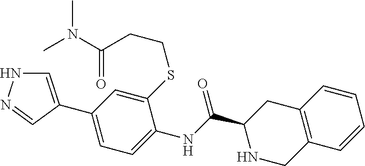

The title compound was prepared according to the procedure described in Scheme 2. ¹H-NMR (DMSO-d₆, 400 MHz), δ: 10.26 (s, 1H), 9.62-9.60 (m, 2H), 8.13-8.12 (m, 2H), 7.75 (s, 1H), 7.58-7.54 (m, 2H), 7.31-7.29 (m, 4H), 4.49-4.32 (m, 3H), 3.54-3.49 (m, 2H), 3.16 (t, J=6.8 Hz, 2H), 2.92 (s, 3H), 2.75 (s, 3H), 2.64 (t, J=6.8 Hz, 2H); LC-MS: single peak at 254 nm, MH⁺ calcd. For C₂₄H₂₇N₅O₂S: 450, obtained: 450.

### Example 56

### (R)—N-(2-(3-(dimethylamino)-3-oxopropylthio)-4-(1H-pyrazol-4-yl)phenyl)-6-methoxy-1,2,3,4-tetrahydroisoquinoline-3-carboxamide

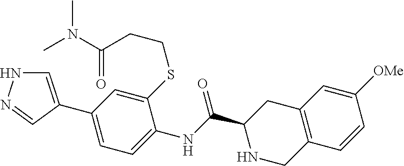

The title compound was prepared according to the procedure described in Scheme 2. LC-MS: single peak at 254 nm, MH⁺ calcd. For C₂₅H₂₉N₅O₃S: 480, obtained: 480.

### Example 57

### N-(4-(1H-pyrazol-4-yl)phenyl)-6-methoxy-1,2,3,4-tetrahydroisoquinoline-3-carboxamide

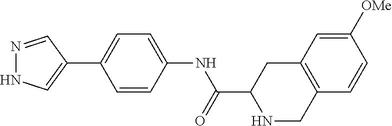

Procedures in Scheme 1 were utilized to synthesize this titled compound. ¹H NMR (400 MHz, MeOH-d4) δ ppm 7.98-7.95 (m, 2H), 7.68-7.53 (m, 4H), 7.17 (d, J=8.5 Hz, 1H), 6.94-6.81 (m, 2H), 4.40 (m, 2H), 4.28 (dd, J=12.1, 4.8 Hz, 1H), 3.79 (s, 3H), 3.46 (dd, J=17.0, 4.8 Hz, 1H), 3.29-3.20 (m, 1H). LC/MS: C₂₀H₂₀N₄O₂ (M+1) 349. Single peak at both 215 nm and 254 nm in analytical HPLC traces.

### Example 58

### N-(4-(1H-pyrazol-4-yl)phenyl)-6-ethoxy-1,2,3,4-tetrahydroisoquinoline-3-carboxamide

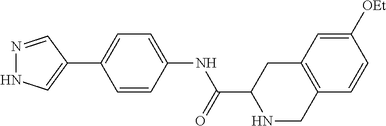

Procedures in Scheme 1 were utilized to synthesize this titled compound. ¹H NMR (400 MHz, MeOH-d4) δ ppm 8.05-7.94 (m, 2H), 7.51-7.42 (m, 4H), 7.19 (d, J=8.5 Hz, 1H), 6.93-6.86 (m, 2H), 4.46-4.37 (m, 3H), 4.06 (q, J=7.0 Hz, 2H), 3.50 (dd, J=17.0, 4.9 Hz, 1H), 3.35-3.25 (m, 1H), 1.40 (t, J=7.0 Hz, 3H). LC/MS: C₂₁H₂₂N₄O₂ (M+1) 363. Single peak at both 215 nm and 254 nm in analytical HPLC traces.

### Example 59

### (R)—N-(4-(1H-pyrazol-4-yl)phenyl)-6-fluoro-1,2,3,4-tetrahydroisoquinoline-3-carboxamide

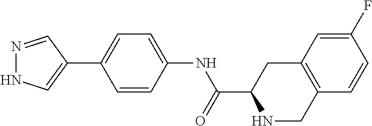

Procedures in Scheme 1 were utilized to synthesize this titled compound. ¹H NMR (400 MHz, DMSO-d6) δ ppm 10.60 (s, 1H), 9.62 (s, 1H), 8.03 (s, 2H), 7.64-7.58 (m, 4H), 7.35 (dd, J=8.5, 5.7 Hz, 1H), 7.24-7.13 (m, 2H), 4.46-4.25 (m, 1H), 3.13 (dd, J=16.8, 12.5 Hz, 1H), 3.00-2.11 (m, 1H). LC/MS: C₁₉H₁₇N₄OF (M+1) 337. Single peak at both 215 nm and 254 nm in analytical HPLC traces.

### Example 60

### (S)—N-(4-(1H-pyrazol-4-yl)phenyl)-6-fluoro-1,2,3,4-tetrahydroisoquinoline-3-carboxamide

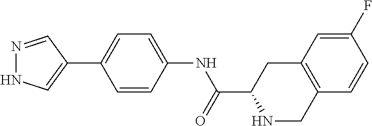

Procedures in Scheme 1 were utilized to synthesize this titled compound. ¹H NMR (400 MHz, DMSO-d6) δ ppm. LC/MS: C₁₉H₁₇N₄OF (M+1) 337. Single peak at both 215 nm and 254 nm in analytical HPLC traces.

### Example 61

### N-(2-(2-(dimethylamino)ethoxy)-4-(1H-pyrazol-4-yl)phenyl)-6-methoxy-1,2,3,4-tetrahydroisoquinoline-3-carboxamide

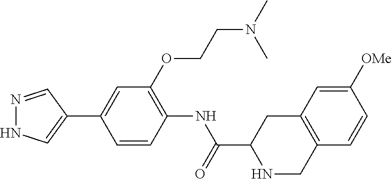

Procedures in Scheme 2 were utilized to synthesize this titled compound. ¹H NMR (400 MHz, DMSO-d6) δ ppm 8.14-8.06 (m, 2H), 7.82-7.77 (m, 1H), 7.41-7.16 (m, 4H), 7.00-6.81 (m, 3H), 4.53-4.25 (m, 4H), 3.87-3.79 (m, 1H), 3.76 (s, 3H), 3.60 (s, 1H), 3.38 (dd, J=17.0, 4.3 Hz, 1H), 3.18-3.04 (m, 1H), 2.94 (s, 6H), 2.90-2.86 (m, 1H), 2.74-2.66 (m, 1H). LC/MS: C₂₄H₂₉N₅O₃ (M+1) 436. Single peak at both 215 nm and 254 nm in analytical HPLC traces.

### Example 62

### N-(2-(2-(dimethylamino)ethoxy)-4-(1H-pyrazol-4-yl)phenyl)-6-ethoxy-1,2,3,4-tetrahydroisoquinoline-3-carboxamide

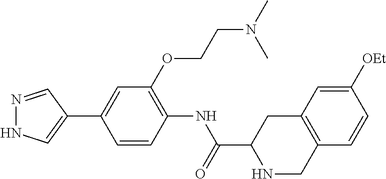

Procedures in Scheme 2 were utilized to synthesize this titled compound. ¹H NMR (400 MHz, DMSO-d6) δ ppm 8.16-8.06 (m, 2H), 7.80-7.75 (m, 1H), 7.37-7.12 (m, 4H), 7.02-6.85 (m, 3H), 4.54-4.24 (m, 4H), 4.06-3.97 (m, 1H), 3.67-3.49 (m, 2H), 3.41-3.32 (m, 1H), 3.16-3.04 (m, 1H), 2.89 (s, 6H), 2.55 (t, J=5.6 Hz, 2H), 1.36-1.26 (m, 3H). LC/MS: C₂₅H₃₁N₅O₃ (M+1) 450. Single peak at both 215 nm and 254 nm in analytical HPLC traces.

### Example 63

### (R)—N-(2-(2-(dimethylamino)ethoxy)-4-(1H-pyrazol-4-yl)phenyl)-6-fluoro-1,2,3,4-tetrahydroisoquinoline-3-carboxamide

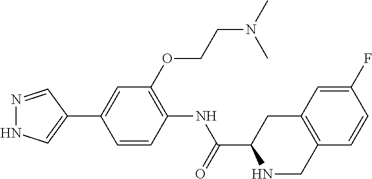

Procedures in Scheme 2 were utilized to synthesize this titled compound. ¹H NMR (400 MHz, MeOH-d4) δ ppm 8.05-7.97 (m, 3H), 7.42-7.04 (m, 5H), 4.62-4.41 (m, 4H), 3.79-3.61 (m, 2H), 3.61-3.43 (m, 1H), 3.06-2.99 (m, 8H). LC/MS: C₂₃H₂₆N₅O₂F (M+1) 424. Single peak at both 215 nm and 254 nm in analytical HPLC traces.

### Example 64

### (S)—N-(2-(2-(dimethylamino)ethoxy)-4-(1H-pyrazol-4-yl)phenyl)-6-fluoro-1,2,3,4-tetrahydroisoquinoline-3-carboxamide

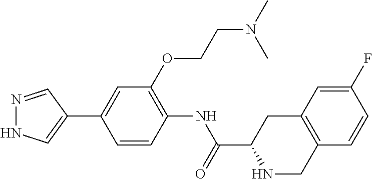

Procedures in Scheme 2 were utilized to synthesize this titled compound. ¹H NMR (400 MHz, MeOH-d4) δ ppm 8.05-7.97 (m, 3H), 7.42-7.04 (m, 5H), 4.62-4.41 (m, 4H), 3.79-3.61 (m, 2H), 3.61-3.43 (m, 1H), 3.06-2.99 (m, 8H). LC/MS: C₂₃H₂₆N₅O₂F (M+1) 424. Single peak at both 215 nm and 254 nm in analytical HPLC traces.

### Example 65

### (R)—N-(2-(2-(dimethylamino)ethoxy)-4-(1H-pyrazol-4-yl)phenyl)-6-methoxy-1,2,3,4-tetrahydroisoquinoline-3-carboxamide

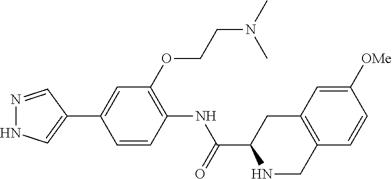

Procedures in Scheme 2 were utilized to synthesize this titled compound. ¹H NMR (400 MHz, DMSO-d6) δ ppm 8.11-8.03 (m, 2H), 7.80-7.73 (m, 1H), 7.40-7.13 (m, 4H), 7.01-6.86 (m, 3H), 4.54-4.25 (m, 4H), 3.87-3.78 (m, 1H), 3.76 (s, 3H), 3.58 (s, 1H), 3.33 (dd, J=17.0, 4.3 Hz, 1H), 3.17-3.04 (m, 1H), 2.96 (s, 6H), 2.92-2.86 (m, 1H), 2.75-2.64 (m, 1H). LC/MS: C₂₄H₂₉N₅O₃ (M+1) 436. Single peak at both 215 nm and 254 nm in analytical HPLC traces.

### Example 66

### (S)—N-(2-(2-(dimethylamino)ethoxy)-4-(1H-pyrazol-4-yl)phenyl)-6-methoxy-1,2,3,4-tetrahydroisoquinoline-3-carboxamide

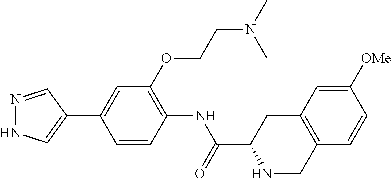

Procedures in Scheme 2 were utilized to synthesize this titled compound. ¹H NMR (400 MHz, MeOH-d4) δ ppm. LC/MS: C₂₄H₂₉N₅O₃ (M+1) 436. Single peak at both 215 nm and 254 nm in analytical HPLC traces.

### Example 67

### (R)—N-(2-(2-(dimethylamino)ethoxy)-4-(3-methyl-1H-pyrazol-4-yl)phenyl)-6-methoxy-1,2,3,4-tetrahydroisoquinoline-3-carboxamide

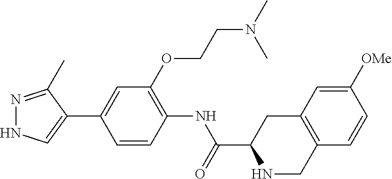

Procedures in Scheme 2 were utilized to synthesize this titled compound. ¹H NMR (400 MHz, MeOH-d4) δ ppm. LC/MS: C₂₅H₃₁N₅O₃ (M+1) 450. Single peak at both 215 nm and 254 nm in analytical HPLC traces.

### Example 68

### (S)—N-(2-(2-(dimethylamino)ethoxy)-4-(3-methyl-1H-pyrazol-4-yl)phenyl)-6-methoxy-1,2,3,4-tetrahydroisoquinoline-3-carboxamide

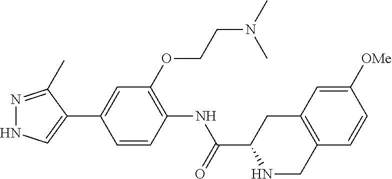

Procedures in Scheme 2 were utilized to synthesize this titled compound. ¹H NMR (400 MHz, MeOH-d4) δ ppm. LC/MS: C₂₅H₃₁N₅O₃ (M+1) 450. Single peak at both 215 nm and 254 nm in analytical HPLC traces.

### Example 69

### (R)—N-(2-(2-(dimethylamino)ethylthio)-4-(1H-pyrazol-4-yl)phenyl)-6-methoxy-1,2,3,4-tetrahydroisoquinoline-3-carboxamide

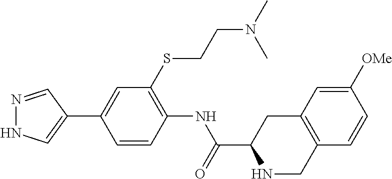

Procedures in Scheme 2 were utilized to synthesize this titled compound. ¹H NMR (400 MHz, MeOH-d4) δ ppm 8.09 (s, 2H), 7.91-7.87 (m, 1H), 7.69-7.59 (m, 2H), 7.24 (d, J=8.2 Hz, 1H), 6.98-6.91 (m, 1H), 4.57-4.50 (m, 1H), 4.47 (s, 2H), 3.84 (s, 3H), 3.59 (dd, J=16.9, 4.7 Hz, 1H), 3.45-3.37 (m, 1H), 3.18-3.07 (m, 1H), 2.93 (s, 6H). LC/MS: C₂₄H₂₉N₅O₂S (M+1) 452. Single peak at both 215 nm and 254 nm in analytical HPLC traces.

### Example 70

### (S)—N-(2-(2-(dimethylamino)ethylthio)-4-(1H-pyrazol-4-yl)phenyl)-6-methoxy-1,2,3,4-tetrahydroisoquinoline-3-carboxamide

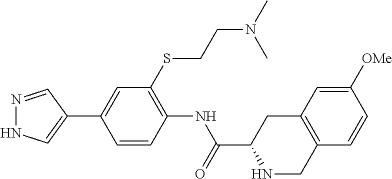

Procedures in Scheme 2 were utilized to synthesize this titled compound. ¹H NMR (400 MHz, MeOH-d4) δ ppm 8.09 (s, 2H), 7.89 (d, J=1.8 Hz, 1H), 7.69-7.58 (m, 2H), 7.23 (d, J=8.4 Hz, 1H), 6.99-6.89 (m, 1H), 4.53 (dd, J=11.8, 5.0 Hz, 1H), 4.46 (s, 2H), 3.84 (s, 3H), 3.65-3.52 (m, 1H), 3.46-3.36 (m, 1H), 2.94-2.82 (m, 6H). LC/MS: C₂₄H₂₉N₅O₂S (M+1) 452. Single peak at both 215 nm and 254 nm in analytical HPLC traces.

### Example 71

### (R)—N-(2-(2-(dimethylamino)ethylthio)-4-(3-methyl-1H-pyrazol-4-yl)phenyl)-6-methoxy-1,2,3,4-tetrahydroisoquinoline-3-carboxamide

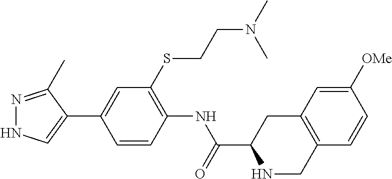

Procedures in Scheme 2 were utilized to synthesize this titled compound. ¹H NMR (400 MHz, MeOH-d4) δ ppm 7.70 (s, 1H), 7.61 (d, J=1.7 Hz, 1H), 7.53 (d, J=8.3 Hz, 1H), 7.40 (dd, J=8.4, 2.0 Hz, 1H), 7.12 (d, J=8.3 Hz, 1H), 6.86-6.79 (m, 2H), 4.44-4.31 (m, 3H), 3.72 (s, 3H), 3.46 (dd, J=16.7, 4.6 Hz, 1H), 3.40-3.26 (m, 1H), 3.05-3.01 (m, 1H), 2.81-2.79 (m, 6H), 2.35 (s, 3H). LC/MS: C₂₅H₃₁N₅O₂S (M+1) 466. Single peak at both 215 nm and 254 nm in analytical HPLC traces.

### Example 72

### (S)—N-(2-(2-(dimethylamino)ethylthio)-4-(3-methyl-1H-pyrazol-4-yl)phenyl)-6-methoxy-1,2,3,4-tetrahydroisoquinoline-3-carboxamide

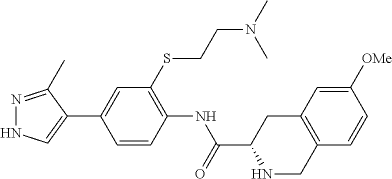

Procedures in Scheme 2 were utilized to synthesize this titled compound. ¹H NMR (400 MHz, MeOH-d4) δ ppm 7.71 (s, 1H), 7.60 (d, J=1.7 Hz, 1H), 7.52 (d, J=8.3 Hz, 1H), 7.40 (m, 1H), 7.09 (d, J=8.3 Hz, 1H), 6.85-6.79 (m, 2H), 4.43-4.30 (m, 3H), 3.74 (s, 3H), 3.43 (dd, J=16.7, 4.6 Hz, 1H), 3.41-3.20 (m, 1H), 3.05-3.00 (m, 1H), 2.80-2.75 (m, 6H), 2.36 (s, 3H). LC/MS: C₂₅H₃₁N₅O₂S (M+1) 466. Single peak at both 215 nm and 254 nm in analytical HPLC traces.

### Example 73

### (3R)—N-(2-(2-(dimethylamino)ethylsulfinyl)-4-(3-methyl-1H-pyrazol-4-yl)phenyl)-6-methoxy-1,2,3,4-tetrahydroisoquinoline-3-carboxamide

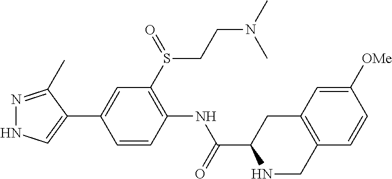

Procedures in Scheme 2 were utilized to synthesize this titled compound. ¹H NMR (400 MHz, MeOH-d4) δ ppm 7.78 (s, 1H), 7.43 (d, J=8.3 Hz, 1H), 7.14-7.03 (m, 2H), 6.86-6.73 (m, 4H), 4.46-4.38 (m, 1H), 4.37-4.29 (m, 1H), 4.25 (s, 2H), 3.72 (s, 3H), 3.58-3.49 (m, 1H), 3.34-3.23 (m, 1H), 2.94 (s, 3H), 2.85 (s, 13H), 2.39 (s, 3H). LC/MS: C₂₅H₃₁N₅O₃S (M+1) 480. Single peak at both 215 nm and 254 nm in analytical HPLC traces.

### Example 74

### (3S)—N-(2-(2-(dimethylamino)ethylsulfinyl)-4-(3-methyl-1H-pyrazol-4-yl)phenyl)-6-methoxy-1,2,3,4-tetrahydroisoquinoline-3-carboxamide

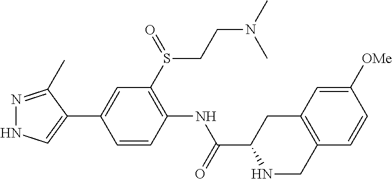

Procedures in Scheme 2 were utilized to synthesize this titled compound. ¹H NMR (400 MHz, MeOH-d4) δ ppm. LC/MS: C₂₅H₃₁N₅O₃S (M+1) 480. Single peak at both 215 nm and 254 nm in analytical HPLC traces.

### Example 75

### (R)-6-methoxy-N-(2-(2-oxo-2-(pyrrolidin-1-yl)ethylthio)-4-(1H-pyrazol-4-yl)phenyl)-1,2,3,4-tetrahydroisoquinoline-3-carboxamide

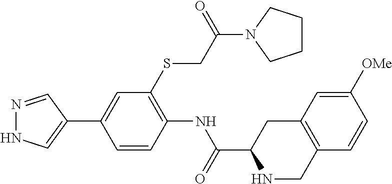

Procedures in Scheme 2 were utilized to synthesize this titled compound. ¹H NMR (400 MHz, MeOH-d4) δ ppm 8.01-7.92 (m, 4H), 7.65-7.60 (m, 1H), 7.21-7.17 (m, 1H), 6.93-6.87 (m, 2H), 4.54 (dd, J=12.0, 4.9 Hz, 1H), 4.43 (s, 2H), 3.82-3.77 (m, 4H), 3.68 (dd, J=17.0, 4.8 Hz, 1H), 3.42-3.34 (m, 4H), 1.92-1.75 (m, 4H). LC/MS: C₂₆H₂₉N₅O₃S (M+1) 492. Single peak at both 215 nm and 254 nm in analytical HPLC traces.

### Example 76

### (R)—N-(2-(2-(dimethylamino)-2-oxoethylthio)-4-(1H-pyrazol-4-yl)phenyl)-6-methoxy-1,2,3,4-tetrahydroisoquinoline-3-carboxamide

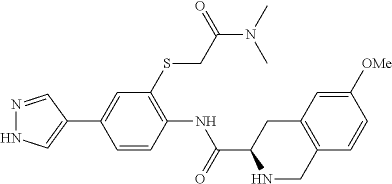

Procedures in Scheme 2 were utilized to synthesize this titled compound. ¹H NMR (400 MHz, MeOH-d4) δ ppm 8.03 (s, 2H), 7.98-7.93 (m, 2H), 7.66-7.62 (m, 1H), 7.25-7.20 (m, 1H), 6.96-6.90 (m, 2H), 4.59-4.51 (m, 1H), 4.45 (s, 2H), 3.94 (d, J=2.4 Hz, 1H), 3.83 (s, 3H), 3.75-3.66 (m, 2H), 3.52-3.37 (m, 2H), 3.04 (s, 3H), 2.96 (s, 3H). LC/MS: C₂₄H₂₇N₅O₃S (M+1) 466. Single peak at both 215 nm and 254 nm in analytical HPLC traces.

### Example 77

### (R)—N-(2-(2-(dimethylamino)ethylthio)-4-(1H-pyrazol-4-yl)phenyl)-6-fluoro-1,2,3,4-tetrahydroisoquinoline-3-carboxamide

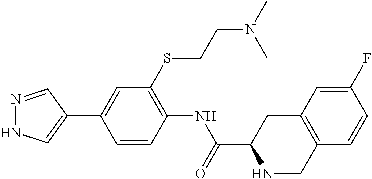

Procedures in Scheme 2 were utilized to synthesize this titled compound. ¹H NMR (400 MHz, MeOH-d4) δ ppm 8.08 (s, 2H), 7.87 (d, J=1.7 Hz, 1H), 7.67-7.57 (m, 2H), 7.35 (dd, J=8.4, 5.5 Hz, 1H), 7.18-7.08 (m, 2H), 4.59-4.47 (m, 3H), 3.61 (dd, J=17.1, 4.7 Hz, 1H), 3.45-3.35 (m, 4H), 2.92 (s, 6H). LC/MS: C₂₃H₂₆N₅OSF (M+1) 440. Single peak at both 215 nm and 254 nm in analytical HPLC traces.

### Example 78

### (S)—N-(2-(2-(dimethylamino)ethylthio)-4-(1H-pyrazol-4-yl)phenyl)-6-fluoro-1,2,3,4-tetrahydroisoquinoline-3-carboxamide

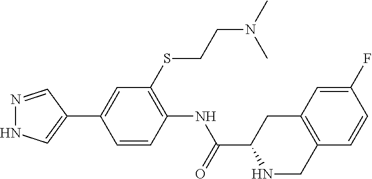

Procedures in Scheme 2 were utilized to synthesize this titled compound. ¹H NMR (400 MHz, MeOH-d4) δ ppm 8.08 (s, 2H), 7.88 (d, J=1.7 Hz, 1H), 7.67-7.57 (m, 2H), 7.35 (dd, J=8.4, 5.4 Hz, 1H), 7.18-7.09 (m, 2H), 4.57-4.49 (m, 3H), 3.66-3.55 (m, 1H), 3.46-3.34 (m, 5H), 2.92 (s, 6H). LC/MS: C₂₃H₂₆N₅OSF (M+1) 440. Single peak at both 215 nm and 254 nm in analytical HPLC traces.

### Example 79

### (3R)—N-(2-(2-(dimethylamino)ethylsulfinyl)-4-(1H-pyrazol-4-ylphenyl)-6-fluoro-1,2,3,4-tetrahydroisoquinoline-3-carboxamide

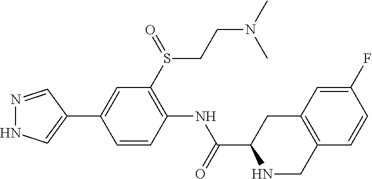

Procedures in Scheme 2 were utilized to synthesize this titled compound. NMR (400 MHz, MeOH-d4) δ ppm 8.17-8.13 (m, 3H), 7.93 (dd, J=8.3, 1.9 Hz, 1H), 7.53 (d, J=8.3 Hz, 1H), 7.36 (dd, J=8.5, 5.4 Hz, 1H), 7.19-7.10 (m, 2H), 4.67-4.37 (m, 3H), 3.74-3.65 (m, 1H), 3.64-3.48 (m, 2H), 3.47-3.36 (m, 1H), 3.01-2.95 (m, 6H). LC/MS: C₂₃H₂₆N₅O₂SF (M+1) 454. Single peak at both 215 nm and 254 nm in analytical HPLC traces.

### Example 80

### (3R)—N-(2-(2-(dimethylamino)ethylsulfinyl)-4-(1H-pyrazol-4-yl)phenyl)-6-fluoro-1,2,3,4-tetrahydroisoquinoline-3-carboxamide

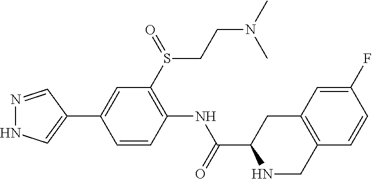

Procedures in Scheme 2 were utilized to synthesize this titled compound. ¹H NMR (400 MHz, MeOH-d4) δ ppm 8.17-8.12 (m, 3H), 7.93 (dd, J=8.2, 2.0 Hz, 1H), 7.53 (d, J=8.3 Hz, 1H), 7.39-7.32 (m, 1H), 7.20-7.10 (m, 2H), 4.58-4.48 (m, 3H), 3.78-3.66 (m, 1H), 3.64-3.44 (m, 2H), 3.43-3.36 (m, 1H), 3.04-2.95 (m, 6H). LC/MS: C₂₃H₂₆N₅O₂SF (M+1) 454. Single peak at both 215 nm and 254 nm in analytical HPLC traces.

### Example 81

### (3S)—N-(2-(2-(dimethylamino)ethylsulfinyl)-4-(1H-pyrazol-4-yl)phenyl)-6-fluoro-1,2,3,4-tetrahydroisoquinoline-3-carboxamide

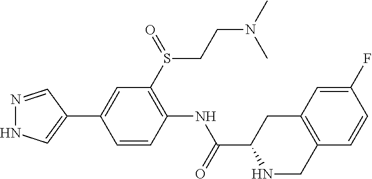

Procedures in Scheme 2 were utilized to synthesize this titled compound. ¹H NMR (400 MHz, MeOH-d4) δ ppm 8.19-8.11 (m, 3H), 7.93 (dd, J=8.3, 1.9 Hz, 1H), 7.53 (d, J=8.3 Hz, 1H), 7.36 (dd, J=8.4, 5.5 Hz, 1H), 7.20-7.10 (m, 2H), 4.69-4.42 (m, 3H), 3.75-3.65 (m, 2H), 3.65-3.47 (m, 2H), 3.47-3.36 (m, 2H), 2.98 (s, 6H). LC/MS: C₂₃H₂₆N₅O₂SF (M+1) 454. Single peak at both 215 nm and 254 nm in analytical HPLC traces.

### Example 82

### (3S)—N-(2-(2-(dimethylamino)ethylsulfinyl)-4-(1H-pyrazol-4-yl)phenyl)-6-fluoro-1,2,3,4-tetrahydroisoquinoline-3-carboxamide

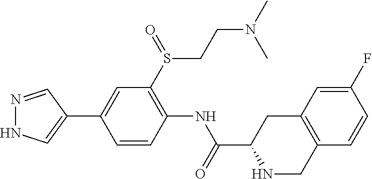

Procedures in Scheme 2 were utilized to synthesize this titled compound. ¹H NMR (400 MHz, MeOH-d4) δ ppm 8.18-8.11 (m, 3H), 7.93 (dd, J=8.3, 2.0 Hz, 1H), 7.51 (d, J=8.3 Hz, 1H), 7.35 (dd, J=8.4, 5.6 Hz, 1H), 7.20-7.09 (m, 2H), 4.56-4.49 (m, 3H), 3.78-3.68 (m, 2H), 3.64-3.44 (m, 2H), 3.41-3.35 (m, 2H), 3.00 (s, 6H). LC/MS: C₂₃H₂₆N₅O₂SF (M+1) 454. Single peak at both 215 nm and 254 nm in analytical HPLC traces.

### Example 83

### (R)—N-(2-carbamoyl-4-(1H-pyrazol-4-yl)phenyl)-6-methoxy-1,2,3,4-tetrahydroisoquinoline-3-carboxamide

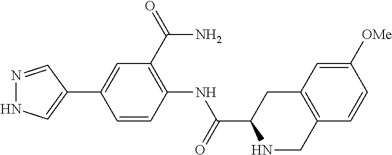

Procedures in Scheme 2 were utilized to synthesize this titled compound. ¹H NMR (400 MHz, MeOH-d4) δ ppm 8.42 (d, J=2.1 Hz, 1H), 8.21-8.08 (m, 3H), 7.84 (d, J=8.4 Hz, 1H), 7.28 (d, J=8.6 Hz, 1H), 7.02-6.92 (m, 2H), 4.61 (dd, J=12.1, 4.7 Hz, 1H), 4.57-4.48 (m, 2H), 3.84 (s, 3H), 3.58 (d, J=4.7 Hz, 1H), 2.74 (s, 1H). LC/MS: C₂₁H₂₁N₅O₃ (M+1) 392. Single peak at both 215 nm and 254 nm in analytical HPLC traces.

### Example 84

### (S)—N-(2-carbamoyl-4-(1H-pyrazol-4-yl)phenyl)-6-methoxy-1,2,3,4-tetrahydroisoquinoline-3-carboxamide

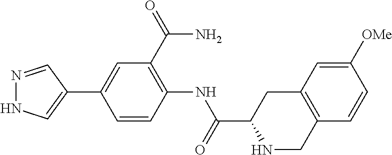

Procedures in Scheme 2 were utilized to synthesize this titled compound. ¹H NMR (400 MHz, MeOH-d4) δ ppm 8.44 (d, J=2.1 Hz, 1H), 8.20-8.08 (m, 3H), 7.82 (d, J=8.5 Hz, 1H), 7.26 (d, J=8.6 Hz, 1H), 6.98-6.89 (m, 2H), 4.63 (dd, J=12.1, 4.7 Hz, 1H), 4.56-4.50 (m, 2H), 3.83 (s, 3H), 3.58 (d, J=4.7 Hz, 1H), 2.71 (s, 1H). LC/MS: C₂₁H₂₁N₅O₃ (M+1) 392. Single peak at both 215 nm and 254 nm in analytical HPLC traces.

### Example 85

### (R)-6-methoxy-N-(2-(2-morpholinoethoxy)-4-(1H-pyrazol-4-yl)phenyl)-1,2,3,4-tetrahydroisoquinoline-3-carboxamide

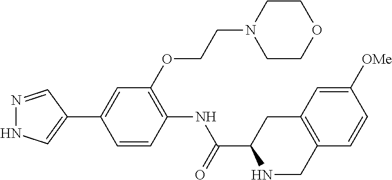

Procedures in Scheme 2 were utilized to synthesize this titled compound. ¹H NMR (400 MHz, MeOH-d4) δ ppm. LC/MS: C₂₆H₃₁N₅O₄ (M+1) 478. Single peak at both 215 nm and 254 nm in analytical HPLC traces.

### Example 86

### (R)-6-methoxy-N-(4-(3-methyl-1H-pyrazol-4-yl)-2-(2-morpholinoethoxy)phenyl)-1,2,3,4-tetrahydroisoquinoline-3-carboxamide

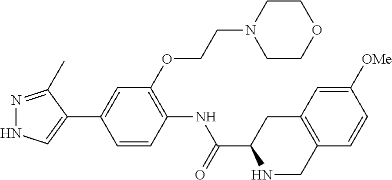

Procedures in Scheme 2 were utilized to synthesize this titled compound. ¹H NMR (400 MHz, MeOH-d4) δ ppm 8.02 (d, J=8.3 Hz, 1H), 7.79 (s, 1H), 7.16-7.00 (m, 3H), 6.89-6.71 (m, 2H), 4.51-4.27 (m, 3H), 3.94-3.83 (m, 1H), 3.84 (s, 3H), 3.64-3.54 (m, 2H), 3.47-3.33 (m, 2H), 2.47 (m, 3H), 2.18 (m, 3H), 1.32 (s, 3H). LC/MS: C₂₇H₃₃N₅O₄ (M+1) 492. Single peak at both 215 nm and 254 nm in analytical HPLC traces.

### Example 87

### (R)-6-methoxy-N-(2-(2-morpholinoethylthio)-4-(1H-pyrazol-4-yl)phenyl)-1,2,3,4-tetrahydroisoquinoline-3-carboxamide

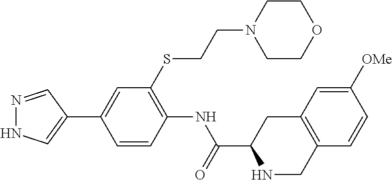

Procedures in Scheme 2 were utilized to synthesize this titled compound. ¹H NMR (400 MHz, MeOH-d4) δ ppm. LC/MS: C₂₆H₃₁N₅O₃S (M+1) 494. Single peak at both 215 nm and 254 nm in analytical HPLC traces.

### Example 88

### (R)-6-methoxy-N-(4-(3-methyl-1H-pyrazol-4-yl)-2-(2-morpholinoethylthio)phenyl)-1,2,3,4-tetrahydroisoquinoline-3-carboxamide

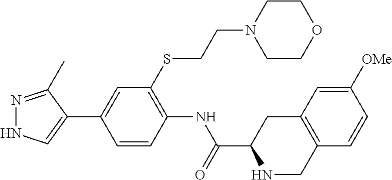

Procedures in Scheme 2 were utilized to synthesize this titled compound. ¹H NMR (400 MHz, MeOH-d4) δ ppm. LC/MS: C₂₇H₃₃N₅O₃S (M+1) 508. Single peak at both 215 nm and 254 nm in analytical HPLC traces.

### Example 89

### (R)—N-(2-(2-hydroxyethoxy)-4-(1H-pyrazol-4-yl)phenyl)-6-methoxy-1,2,3,4-tetrahydroisoquinoline-3-carboxamide

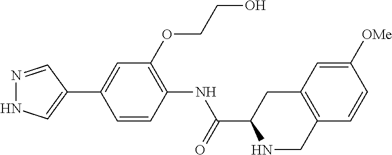

Procedures in Scheme 2 were utilized to synthesize this titled compound. ¹H NMR (400 MHz, MeOH-d4) δ ppm. LC/MS: C₂₂H₂₄N₄O₄ (M+1) 409. Single peak at both 215 nm and 254 nm in analytical HPLC traces.

### Example 90

### (R)—N-(2-(2-(dimethylamino)ethylthio)-4-(1H-pyrazol-4-yl)phenyl)piperidine-2-carboxamide

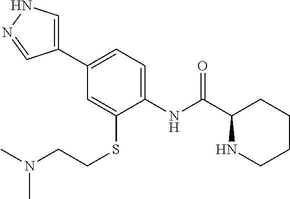

Procedures in Scheme 2 were utilized to synthesize this titled compound. ¹H NMR (400 MHz, MeOH-d4) δ ppm. LC/MS: C₁₉H₂₇N₅OS (M+1) 374. Single peak at both 215 nm and 254 nm in analytical HPLC traces.

### Example 91

### (R)-2-amino-N-(2-(2-(dimethylamino)ethylthio)-4-(1H-pyrazol-4-yl)phenyl)propanamide

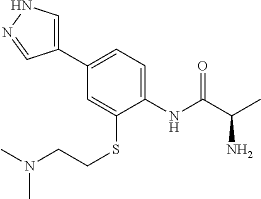

Procedures in Scheme 2 were utilized to synthesize this titled compound. ¹H NMR (400 MHz, MeOH-d4) δ ppm. LC/MS: C₁₆H₂₃N₅OS (M+1) 334. Single peak at both 215 nm and 254 nm in analytical HPLC traces.

### Example 92

### (R)—N-(2-(3-hydroxypropoxy)-4-(1H-pyrazol-4-yl)phenyl)-6-methoxy-1,2,3,4-tetrahydroisoquinoline-3-carboxamide

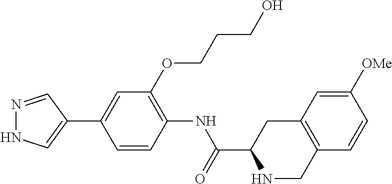

Procedures in Scheme 2 were utilized to synthesize this titled compound. ¹H NMR (400 MHz, MeOH-d4) δ ppm. LC/MS: C₂₃H₂₆N₄O₄ (M+1) 423. Single peak at both 215 nm and 254 nm in analytical HPLC traces.

### Example 93

### (R)—N-(2-((2-(dimethylamino)ethyl)(methyl)amino)-4-(1H-pyrazol-4-yl)phenyl)-6-methoxy-1,2,3,4-tetrahydroisoquinoline-3-carboxamide

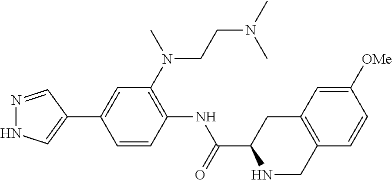

Procedures in Scheme 2 were utilized to synthesize this titled compound. ¹H NMR (400 MHz, MeOH-d4) δ ppm. LC/MS: C₂₅H₃₂N₆O₂ (M+1) 449. Single peak at both 215 nm and 254 nm in analytical HPLC traces.

The following compounds were also prepared using procedures in Scheme 2:

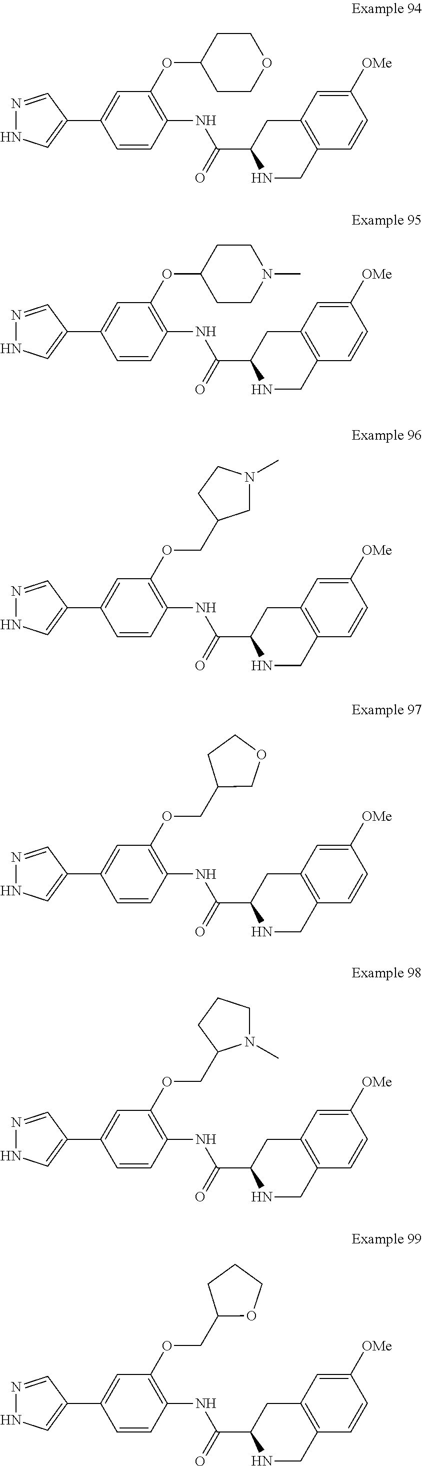

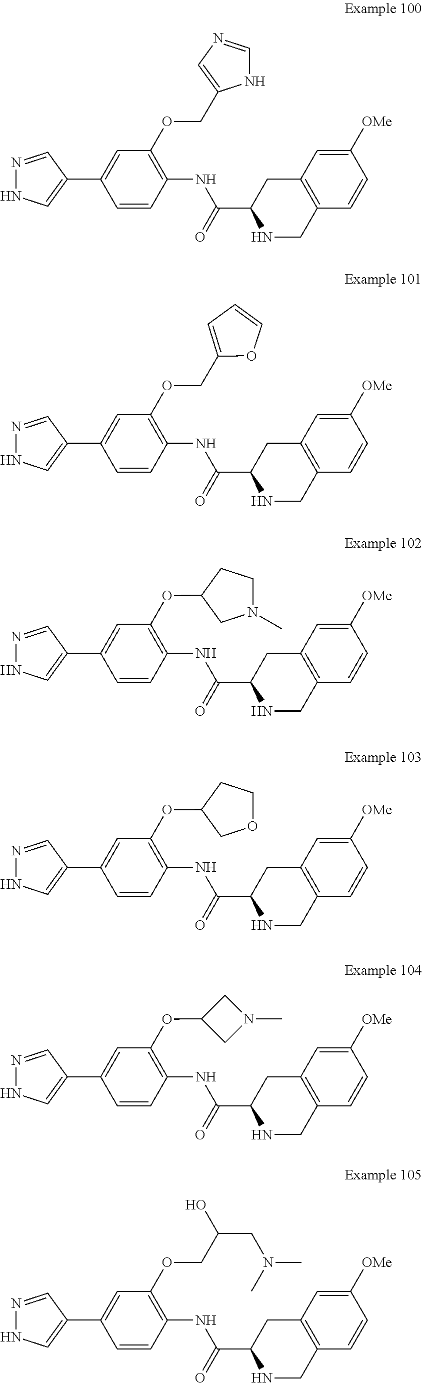

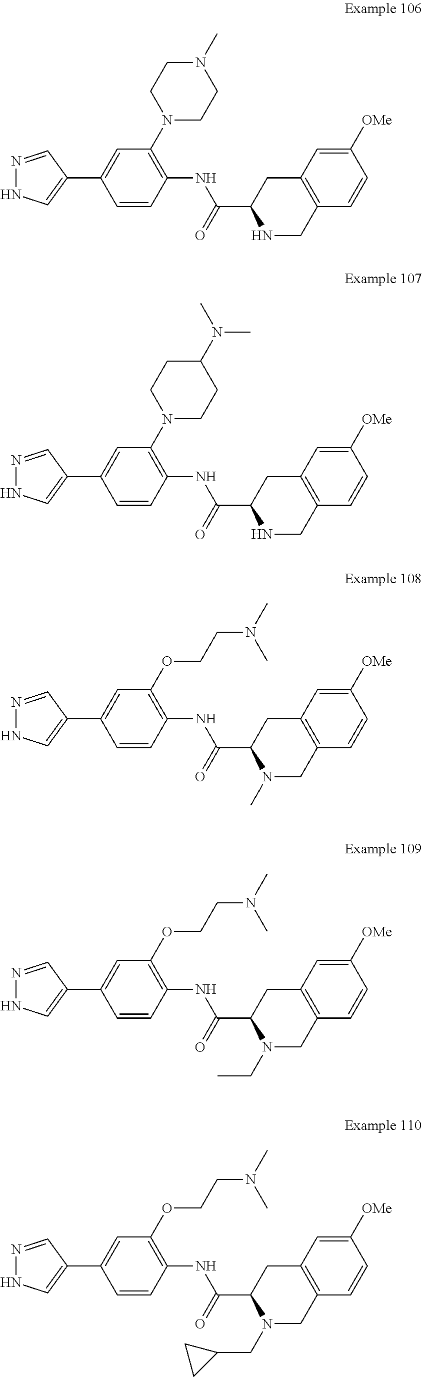

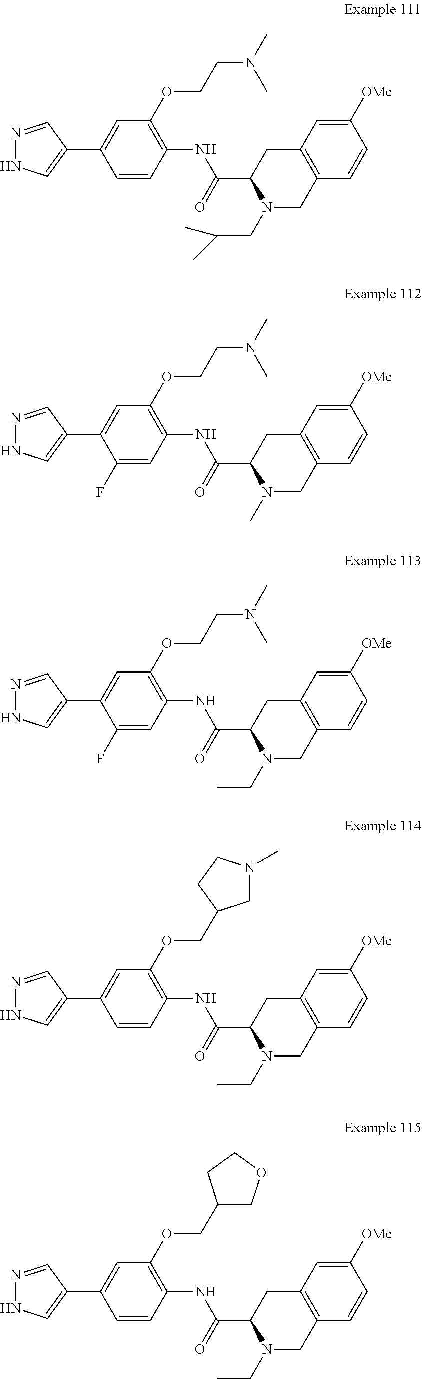

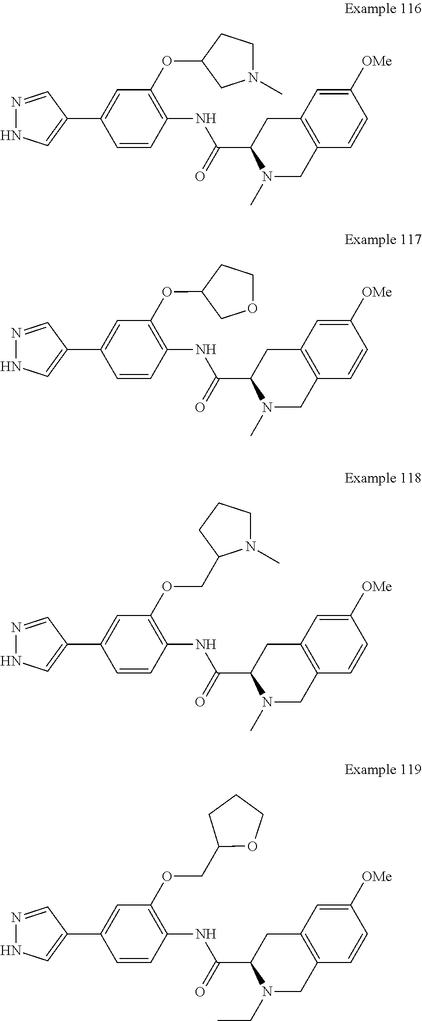

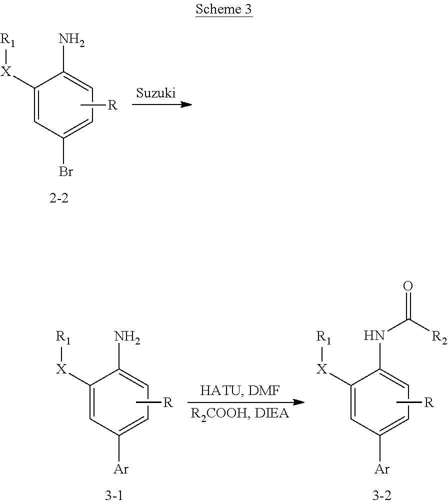

Compound 2-2 was sugjected to a standard Suzuki coupling procedure to obtain compound 3-1. Thus, Pd[P(Ph)₃]₄ (10% by molarity) was added to a degassed solution of 2-2 (1.0 equiv), a suitable boronic acid or ester (1.3 equiv), and K₂CO₃ (3 equiv) in THF/water (4:1 by volume) in a sealed tube or flask. The resulting suspension and heated at 90-100° C. for 2 h or till the Suzuki coupling was finished as monitored by LC-MS. After aqueous work up, the residue was subjected to flash chromatography to give compound 3-1. An amide formation using HATU as the coupling reagent in DMF was then used to obtain compound 3-2, which was purified using preparative HPLC.

### Example 120

### N-(2-(2-(dimethylamino)ethoxy)-4-(1H-pyrazol-4-yl)phenyl)-2-(4-fluorophenyl)-2-hydroxyacetamide

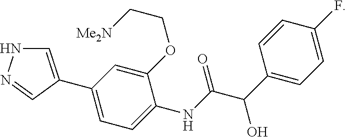

The title compound was prepared according to the procedure described in Scheme 3. LC-MS: single peak at 254 nm, MH⁺ calcd. For C₂₁H₂₃FN₄O₃: 399, obtained: 399.

### Example 121

### N-(2-(2-(dimethylamino)ethoxy)-4-(pyridin-4-yl)phenyl)-2-(4-fluorophenyl)-2-hydroxyacetamide

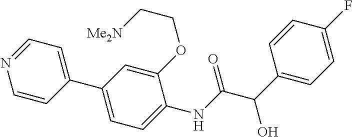

The title compound was prepared according to the procedure described in Scheme 3. LC-MS: single peak at 254 nm, MH⁺ calcd. For C₂₃H₂₄FN₃O₃: 410, obtained: 410.

### Example 122

### N-(4-(2-aminopyrimidin-4-yl)-2-(2-(dimethylamino)ethoxy)phenyl)-2-(4-fluorophenyl)-2-hydroxyacetamide

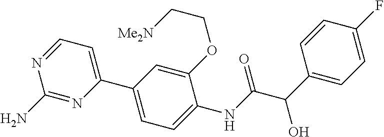

The title compound was prepared according to the procedure described in Scheme 3. LC-MS: single peak at 254 nm, MH⁺ calcd. For C₂₂H₂₄FN₅O₃: 426, obtained: 426.

### Example 123

### N-(4-(1H-pyrazol-4-yl)-2-(tetrahydro-2H-pyran-4-yloxy)phenyl)-2-(4-fluorophenyl)-2-hydroxyacetamide

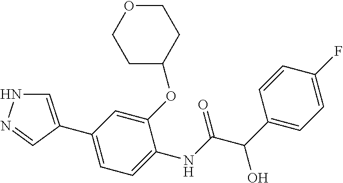

The title compound was prepared according to the procedure described in Scheme 3. LC-MS: single peak at 254 nm, MH⁺ calcd. For C₂₂H₂₂FN₃O₄: 412, obtained: 412.

### Example 124

### 2-(4-fluorophenyl)-2-hydroxy-N-(2-(1-methylpiperidin-4-yloxy)-4-(1H-pyrazol-4-yl)phenyl)acetamide

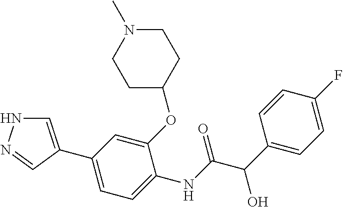

The title compound was prepared according to the procedure described in Scheme 3. LC-MS: single peak at 254 nm, MH⁺ calcd. For C₂₃H₂₅FN₄O₃: 425, obtained: 425.

The following compounds were prepared using procedures in either Scheme 3 or Scheme 2:

| Example | Structure | Formula | MW |
|---|---|---|---|
| 125 | 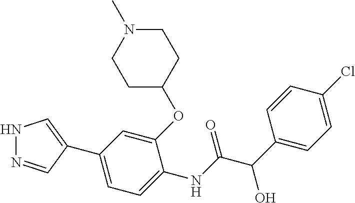 | C₂₃H₂₅ClN₄O₃ | 440 |
| 126 | 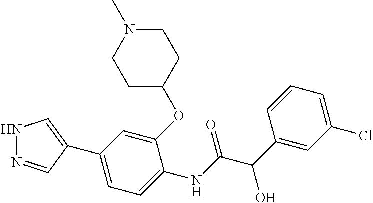 | C₂₃H₂₅ClN₄O₃ | 440 |
| 127 | 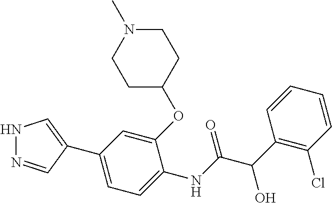 | C₂₃H₂₅ClN₄O₃ | 440 |
| 128 | 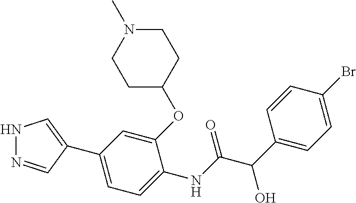 | C₂₃H₂₅BrN₄O₃ | 485 |
| 129 | 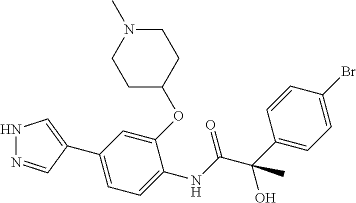 | C₂₄H₂₇BrN₄O₃ | 499 |
| 130 | 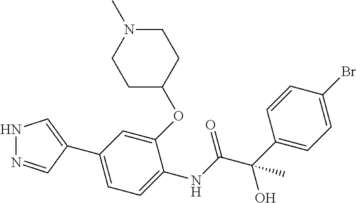 | C₂₄H₂₇BrN₄O₃ | 499 |
| 131 | 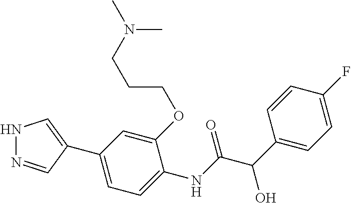 | C₂₂H₂₅FN₄O₃ | 412 |
| 132 | 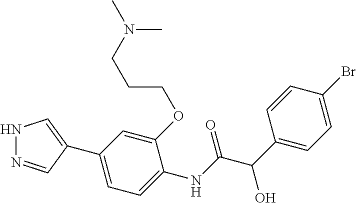 | C₂₂H₂₅BrN₄O₃ | 472 |
| 133 | 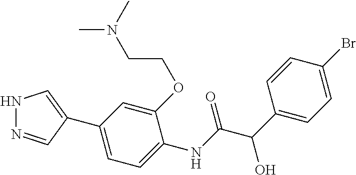 | C₂₁H₂₃BrN₄O₃ | 458 |
| 134 | 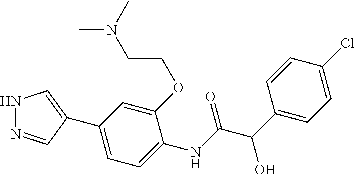 | C₂₁H₂₃ClN₄O₃ | 414 |
| 135 | 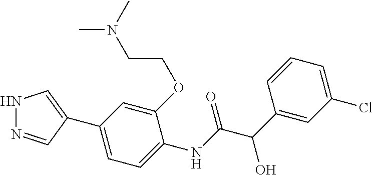 | C₂₁H₂₃ClN₄O₃ | 414 |
| 136 | 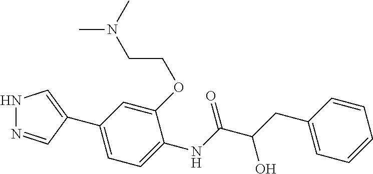 | C₂₂H₂₆N₄O₃ | 394 |
| 137 | 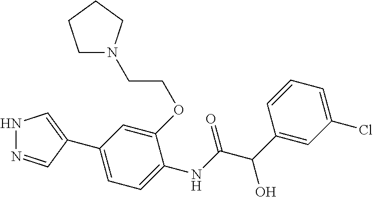 | C₂₃H₂₅ClN₄O₃ | 440 |
| 138 | 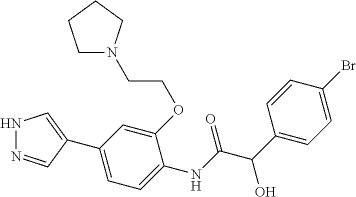 | C₂₁H₂₅BrN₄O₃ | 484 |
| 139 | 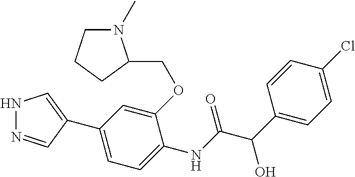 | C₂₃H₂₅ClN₄O₃ | 440 |
| 140 | 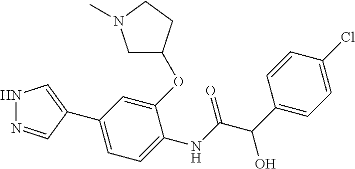 | C₂₂H₂₃ClN₄O₃ | 426 |
| 141 | 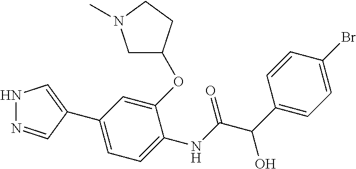 | C₂₂H₂₃BrN₄O₃ | 470 |
| 142 | 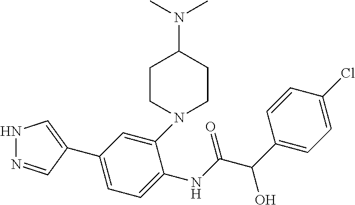 | C₂₄H₂₈ClN₅O₂ | 453 |
| 143 | 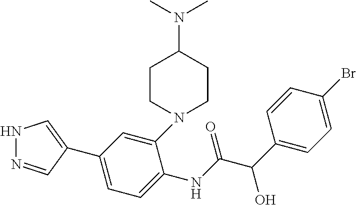 | C₂₄H₂₈BrN₅O₂ | 497 |
| 144 | 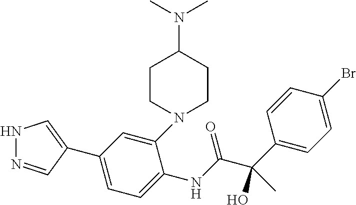 | C₂₅H₃₀BrN₅O₂ | 511 |
| 145 | 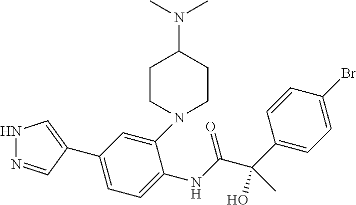 | C₂₅H₃₀BrN₅O₂ | 511 |
| 146 | 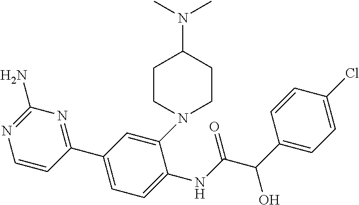 | C₂₅H₂₉ClN₆O₂ | 480 |

### Enzymatic Rho Kinase (ROCK I and ROCK II) Assays.

The assay is based on ability of Rhok2 to phosphorylate a specific peptide sequence derived from its substrate—ribosomal protein S6 (amino acid residues 229-239). Rhok2 uses ATP as a donor of phosphate for the phosphorylation of the substrate, which leads to the depletion of ATP in the reaction mix. An assay kit (“Kinase-Glo”, Promega) was used to quantify enzyme activity. Using this kit, residual amounts of ATP are measured by a secondary enzymatic reaction, through which luciferase utilizes the remaining ATP to produce luminescence. Luminescent signal is directly proportional to ATP concentration and inversely proportional to Rhok2 activity.

This dose response assay was conducted in 1536 well plate format. Each concentration was tested nominally in triplicate. Protocol Summary: A 5 μL mixture of a 20 μM S6-peptide (LCB-AKRRRLSSLRA-NH₂) and 20 μM ATP in Kinase buffer (50 mM Hepes pH 7.3, 10 mM MgCl₂, 0.1% BSA, and 2 mM DTT) was added to the well of a plate. 20 nL of 90% DMSO/10% water containing the compound(s) to be tested (or no compound for control wells) was added. The reaction was initiated by addition of 5 μL of 8 nM ROCK-II (1-543) in Kinase buffer. The reaction was incubated for 50 min at RT and then terminated by addition of 10 μL Kinase-Glo (Promega) reagent. The plate was read after 10 min incubation time at RT on the Viewlux in luminescence mode.

Myosin Light Chain Double Phosphorylation Assays (ppMLC Cell Assay).

A7r5 cells were plated at 5000 cells/well in a 96 well Packard View Plate (Perkin Elmer) in DMEM +10% FBS. After attachment overnight, cells were serum starved for 4 h and treated with inhibitor in 0.25% DMSO final concentration for 1 h at 37° C. Cells were then treated with 10 μM lysophosphatitic acid (LPA) for 10 min. Following treatment, cells were immediately fixed with 4% paraformaldehyde for 30 minutes. After a brief wash in 0.1 M glycine, cells were permeablized in 0.2% Triton X for 10 minutes. Cells were then washed once in PBS and blocked in Li-COR blocking buffer (Li-COR Biosciences) for 1 h at 25 C. Cells were probed for either phosphorylated myosin light chain 20 (55 ng/mL), total myosin light chain (525 ng/mL) or anti-bovine α-tubulin (1 μg/mL), and incubated overnight at 4° C. Following three washes, cells were probed with goat-anti-rabbit or goat-anti-mouse IR800 (2 μg/mL in LI-COR block +0.025% Tween) for 1 h at 25° C. Nuclei were stained with TO-PRO-3 iodide (642/661) (1:4000) for 20 min, washed twice in PBS/0.05% Tween and read with an Odyssey Infrared Imaging System (LI-COR Biosciences).

### Neurite Length Assay (N2a, Cell Assay).

N2a cells are maintained in DMEM/FBS at 37 C and 5% CO₂. For the experiment the cells were plated on a poly-D-lysine coated 96-well tissue culture plate. After attachment, cell differentiation was induced for 2 days by addition of 10 uM retinoic acid. Cells were treated for 1 h with a dilution of compounds in 0.3% DMSO final concentration before neurite retraction was induced by 5 uM LPA. Cells were stained for tubulin and nuclei and images were acquired on a INCell 1000 workstation. Images were analyzed using the developer toolbox and neurite length was quantitated. The IC50s of all examples selected for testing were in the range of 1 nM-1 μM.

Compounds tested in the assays described herein are considered to be active if they exhibit an IC₅₀ of ≦10 μM. Additional examples of activity include IC₅₀'s of ≦1 μM, ≦0.1 μM, ≦0.01 μM, and of ≦0.001 μM. Using the methodology described herein, a number of compounds of the present invention were found to exhibit IC₅₀'s of ≦10 μM, thereby confirming the utility of the compounds of the present invention as effective ROCK inhibitors. Those compounds with IC₅₀ₛ below 150 nM were also tested in the cell assays. The IC₅₀s obtained from cell assays were in the range from 1 nM to 10 uM. For example, the IC50s of Example 26 in enzymatic assay, ppMLC assay, and N2a assay are 19 nM, 110 nM, and 18 nM respectively.

All references cited herein are incorporated by reference. The present invention may be embodied in other specific forms without departing from the spirit or essential attributes thereof and, accordingly, reference should be made to the appended claims, rather than to the foregoing specification, as indicating the scope of the invention.

What is claimed is:

1. A compound of formula I: 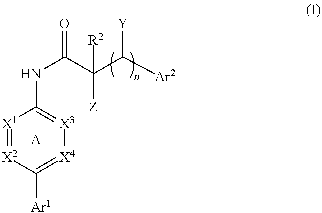
wherein
in ring A comprising each of X¹, X², X³, and X⁴, each of X¹, X², X³, and X⁴ is independently CH, CR¹ or N, provided that no more that two of X¹, X², X³, and X⁴ are N;
R¹ is independently at each occurrence F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS(O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(4-10 membered)-heterocyclyl substituted with 0-2 R^{a} comprising 1-4 heteroatoms selected from O, S(O)_{q}, and N; or two adjacent R¹ substituents can form a fused phenyl or a 5-6 membered heteroaryl comprising carbon atoms and 1-2 heteroatoms selected from O, S(O)_{q}, and N, and substituted with 0-3 R^{a}, wherein p is 0-4 and q is 0-2;
R is independently at each occurrence H, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (3-10 membered)-cycloalkyl substituted with 0-2 R^{a}, or (3-10 membered)-heterocyclyl substituted with 0-2 R^{a} comprising 1-4 heteroatoms selected from O, S(O)_{q}, and N; or, an NR₂ forms a (3-10 membered)-heterocyclyl substituted with 0-2 R^{a} and comprising 0-1 additional ring heteroatoms selected from N, O, and S(O)_{q};
R^{a} is independently at each occurrence oxo, F, Cl, Br, I, CF₃, OCF₃, (C₁₋₆)-alkyl substituted with 0-2 R^{a}, (C₂₋₆)-alkenyl substituted with 0-2 R^{a}, (C₂₋₆)-alkynyl substituted with 0-2 R^{a}, (CH₂)ₚNO₂, (CH₂)ₚCN, (CH₂)ₚOR, (CH₂)ₚNR₂, (CH₂)ₚC(═O)R, (CH₂)ₚOC(═O)R, (CH₂)ₚC(═O)OR, (CH₂)ₚC(═O)NR₂, (CH₂)ₚOC(═O)NR₂, (CH₂)ₚNRC(═O)R, (CH₂)ₚNRC(═O)OR, (CH₂)ₚNRC(═O)NR₂, (CH₂)ₚC(═NH)NH₂, (CH₂)ₚS (O)_{q}R, (CH₂)ₚSO₂NR₂, (CH₂)ₚNRSO₂R, (CH₂)ₚNRSO₂NR₂, (CH₂)ₚ-(3-10 membered)-cycloalkyl substituted with 0-2 R^{a}, or (CH₂)ₚ-(3-10 membered)-heterocyclyl substituted with 0-2 R^{a} comprising 1-4 heteroatoms selected from O, S(O)_{q}, and N;
Ar¹ comprises a 5- or 6-membered heteroaryl comprising at least one nitrogen atom and 0-3 additional heteroatoms selected from O, S(O)_{q}, and N; when Ar¹ is a 5-membered heteroaryl, a nitrogen atom is disposed one atom away from an atom of the heteroaryl bonded to ring A, and when Ar¹ is a 6-membered heteroaryl, a nitrogen atom is disposed two atoms away from an atom of the heteroaryl bonded to ring A; wherein Ar¹ is optionally fused with phenyl or a 5-6 membered heteroaryl comprising 1-2 heteroatoms selected from O, S(O)_{q}, and N, wherein the fused phenyl or 5-6 membered heteroaryl is substituted with 0-3 R^{a};
provided that when Ar¹ comprises pyrazolyl, pyridyl, or pyrimidyl, then ring A is substituted with at least one R¹ which is other than unsubstituted alkyl;
R^{b} is independently at each occurrence H, (C₁₋₆)-alkyl, (C₃₋₆)-cycloalkyl, (C₁₋₆)alkyl-(C₃₋₆)-cycloalkyl, (3-8 membered)-heterocyclyl, (C₁₋₆)alkyl-(3-8 membered)-heterocyclyl, aryl, heteroaryl, aralkyl, or heteroarylalkyl, wherein any R^{b} other than H is substituted with 0-3 R^{a};
R² is H or (C₁₋₆)-alkyl;
n is 0 or 1;
and when n=1, Y is H,
or Y comprising —CH₂— together with Z comprising —CH₂NR^{b}—, and carbon atoms to which Y and Z are bonded can together form a 5- or 6-membered heterocyclyl ring substituted with 0-3 R^{a}, or
when n=0 or 1, Z comprises NH₂ or OH; and,
Ar² comprises aryl, heteroaryl, or is absent, wherein any aryl or heteroaryl is substituted with 0-3 R^{a},
or when Ar² is aryl or heteroaryl, Ar² together with Z comprising NR^{b} can together form an aryl- or heteroaryl-fused 5- or 6-membered heterocyclyl substituted with 0-3 R^{a};
or any salt, stereoisomer, tautomer, hydrate, solvate, or prodrug thereof.

2. The compound of claim 1 wherein Z is NH₂, n=0, and Ar² comprises an aryl or heteroaryl substituted with 0-3 R^{a}, or is absent.

3. The compound of claim 1 wherein Z is OH, n=0, Ar² comprises an aryl or heteroaryl substituted with 0-3 R^{a}, or is absent.

4. The compound of claim 1 wherein n=0 and Ar² together with Z comprising NR² form an aryl- or heteroaryl-fused 5- or 6-membered heterocyclyl substituted with 0-3 R^{a}.

5. The compound of claim 1 wherein Z is NH₂, n=1, and Ar² comprises an aryl or heteroaryl substituted with 0-3 R^{a}.

6. The compound of claim 1 wherein n=1, and Y comprising —CH₂— together with Z comprising —CH₂NR^{b}—, and carbon atoms to which Y and Z are bonded can together form a 5- or 6-membered heterocyclyl ring substituted with 0-3 R^{a}.

7. The compound of claim 1 wherein Ar¹ comprises 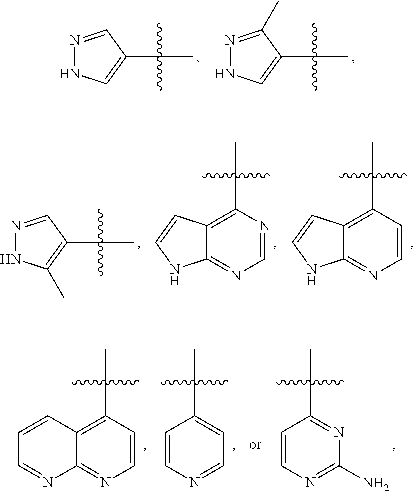
wherein a wavy line indicates a point of attachment.

8. The compound of claim 1 wherein ring A comprises a phenyl, pyridyl, or pyridazinyl ring.

9. The compound of claim 1 comprising a compound of formula Ia-If: 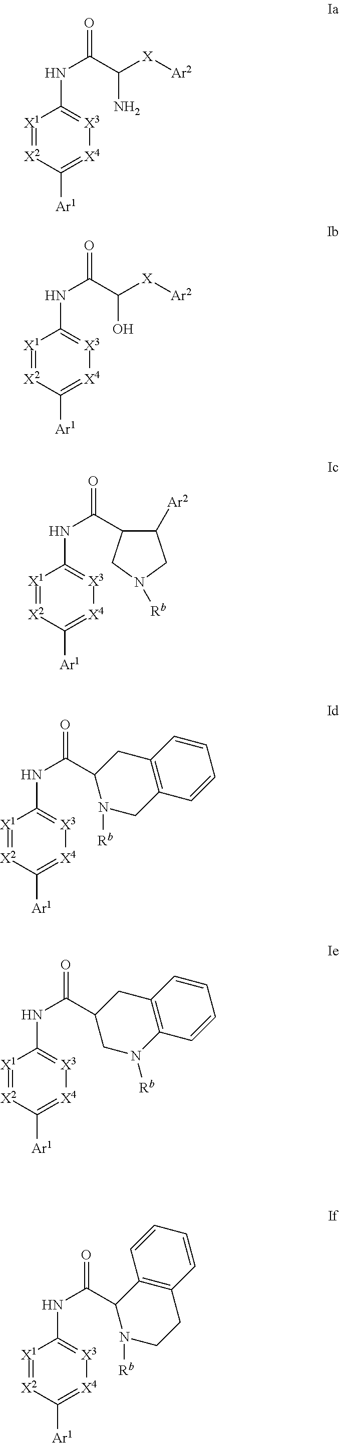
wherein X is absent or is CH₂, or any salt, stereoisomer, tautomer, hydrate, solvate, or prodrug thereof.

10. The compound of claim 1 wherein each independently selected R¹ is chloro, fluoro, methoxy, —C(O)NR₂, —O(CH₂)ₚNR₂, —S(O)_{q}(CH₂)ₚC(O)NR₂, —S(O)_{q}(CH₂)ₚNR₂, —O(CH₂)ₚOR^{a}, N(R)(CH₂)ₚNR₂, —OCH(OH)CH₂NR₂, 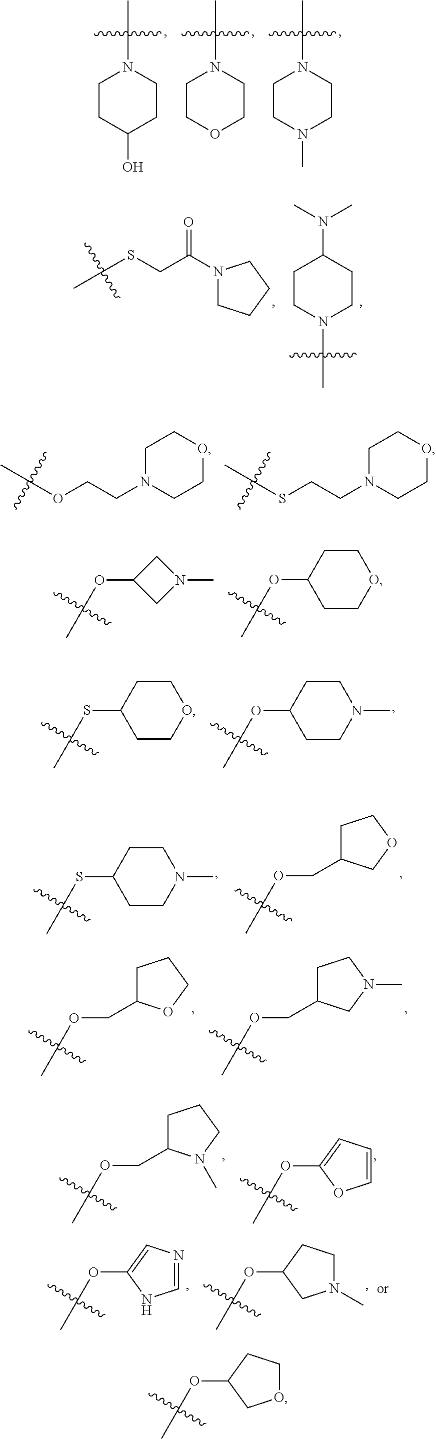
wherein a wavy line indicates a point of attachement.

11. The compound of claim 10 wherein R is methyl, p is 2 or 3, or any combination thereof.

12. The compound of claim 1 wherein the compound is any of the following: 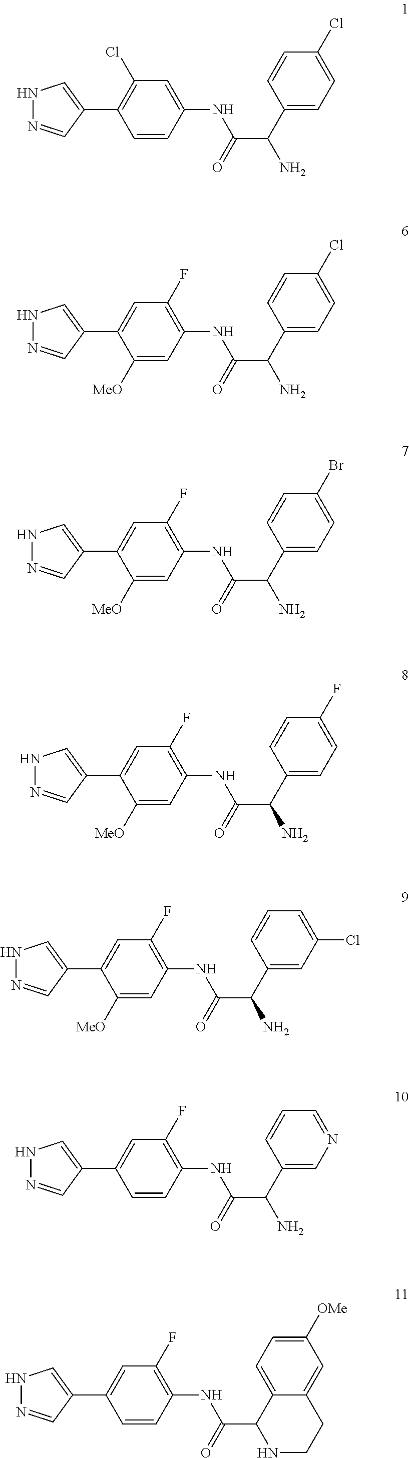 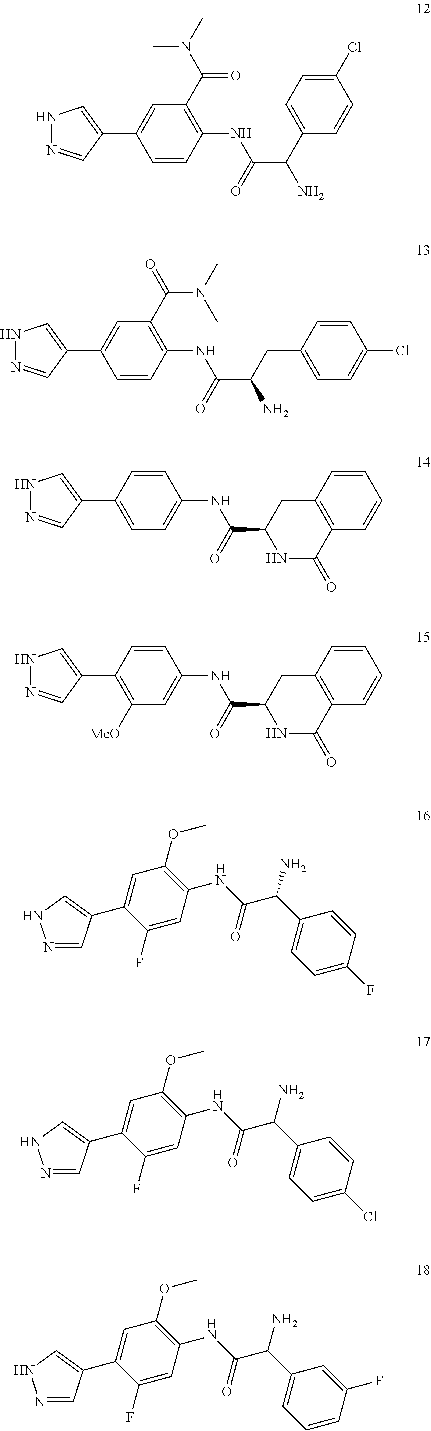 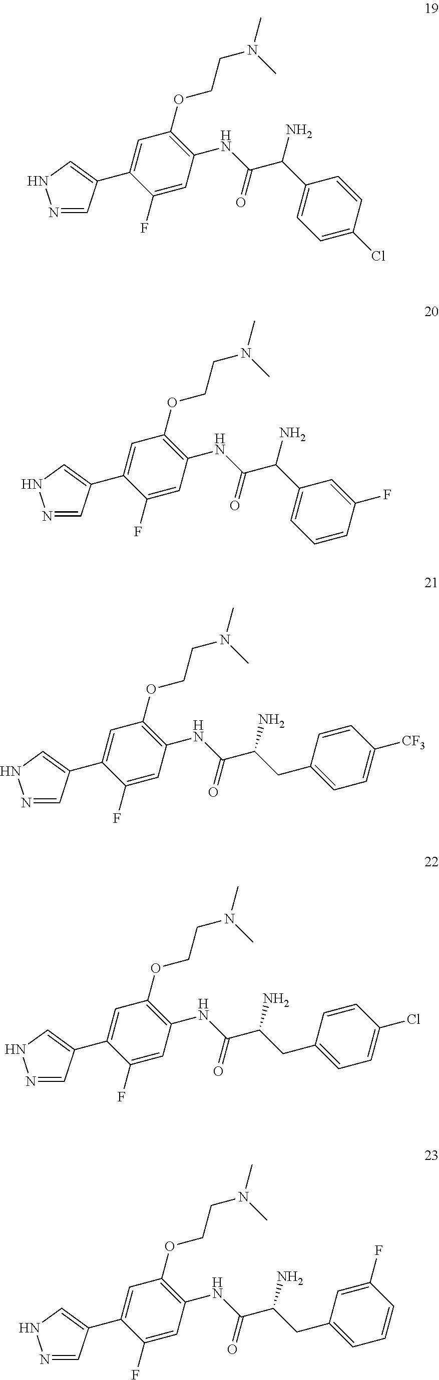 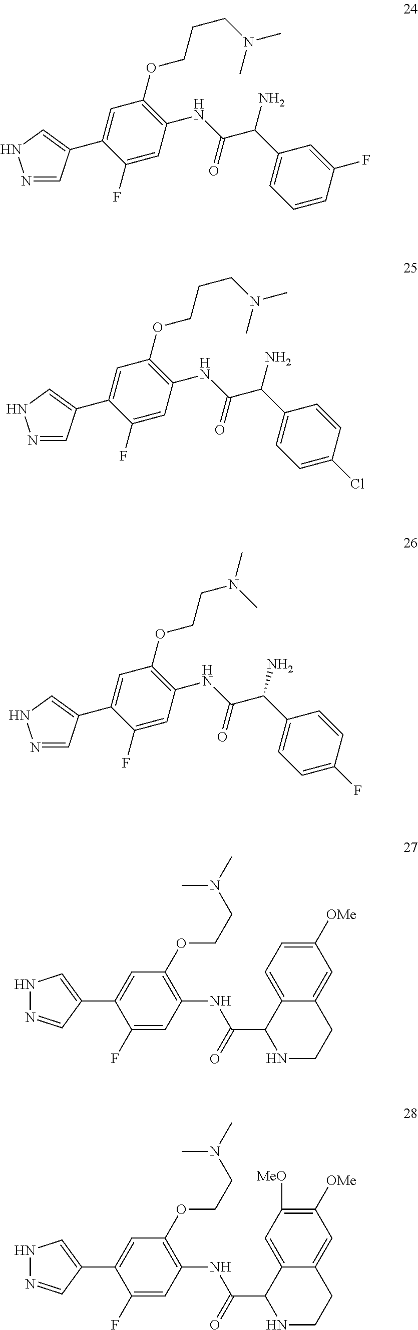 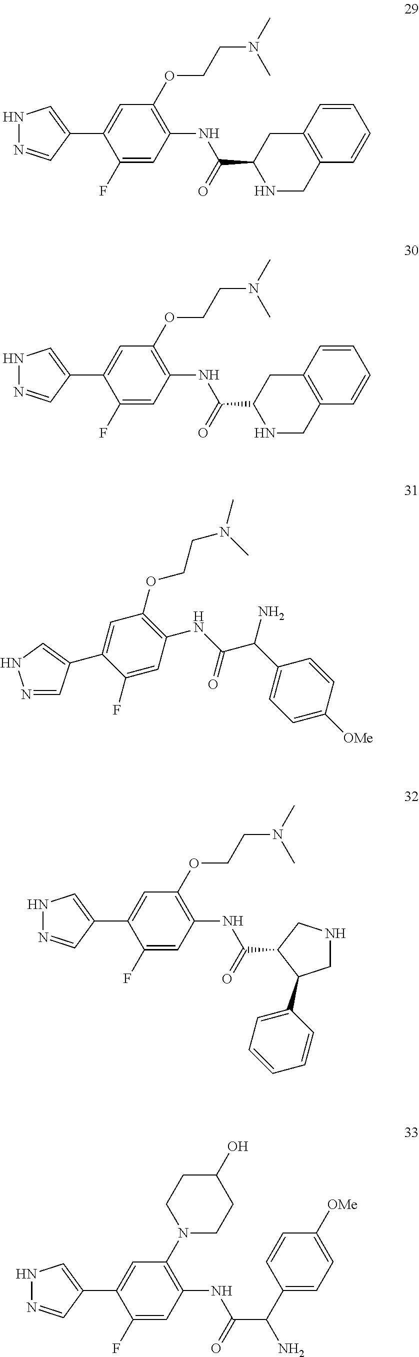 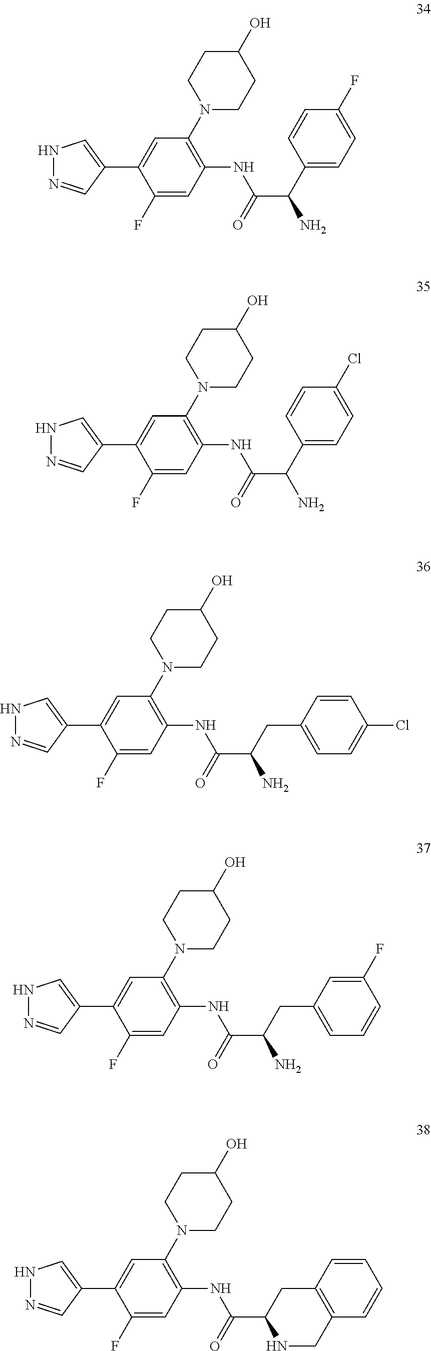 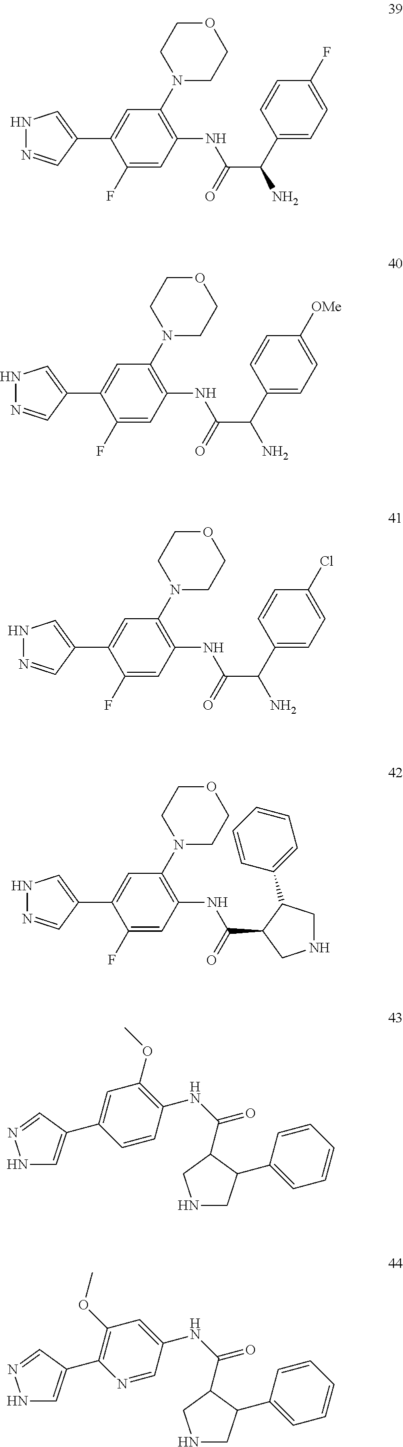 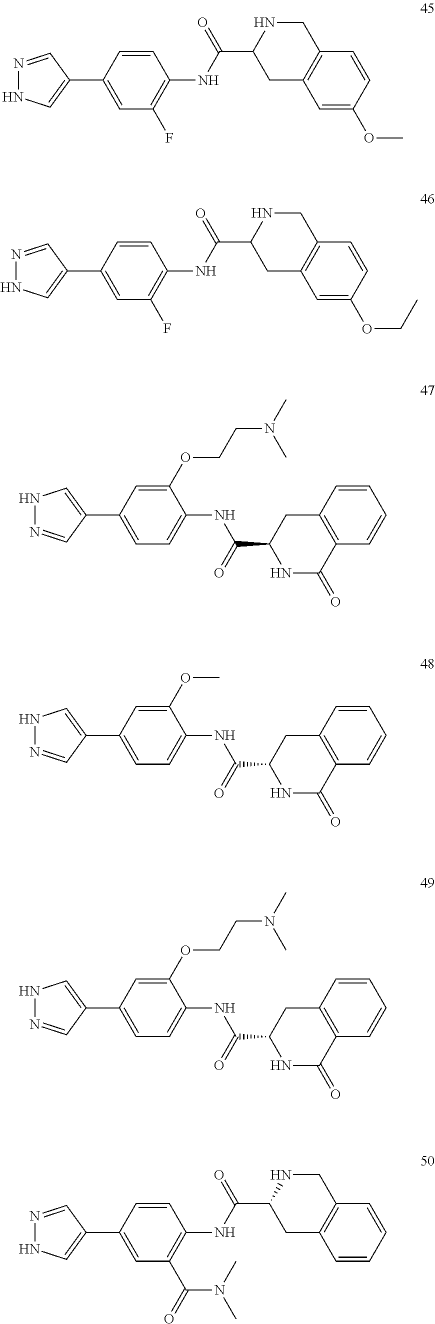 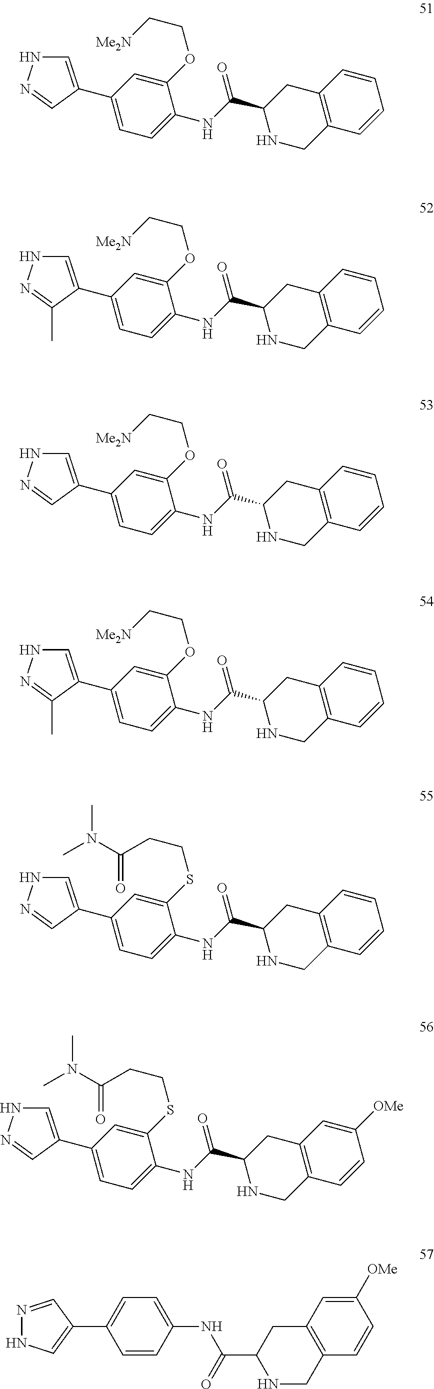 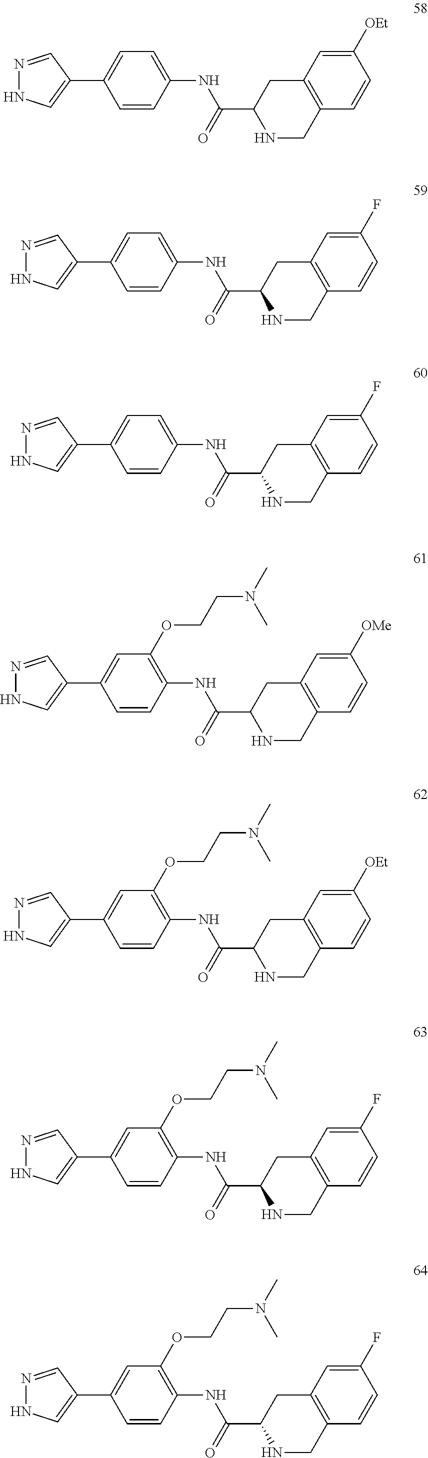 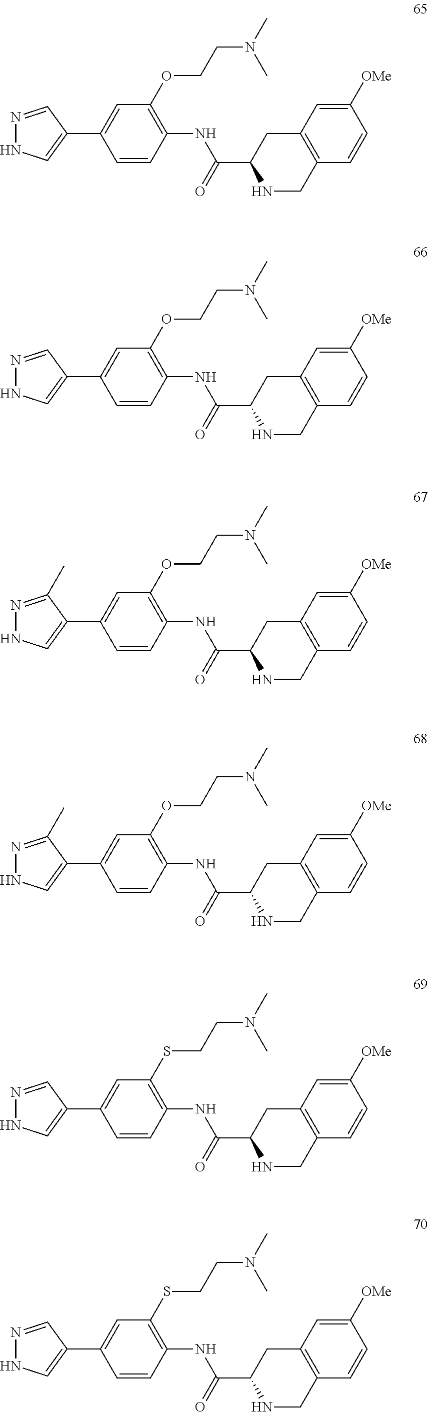 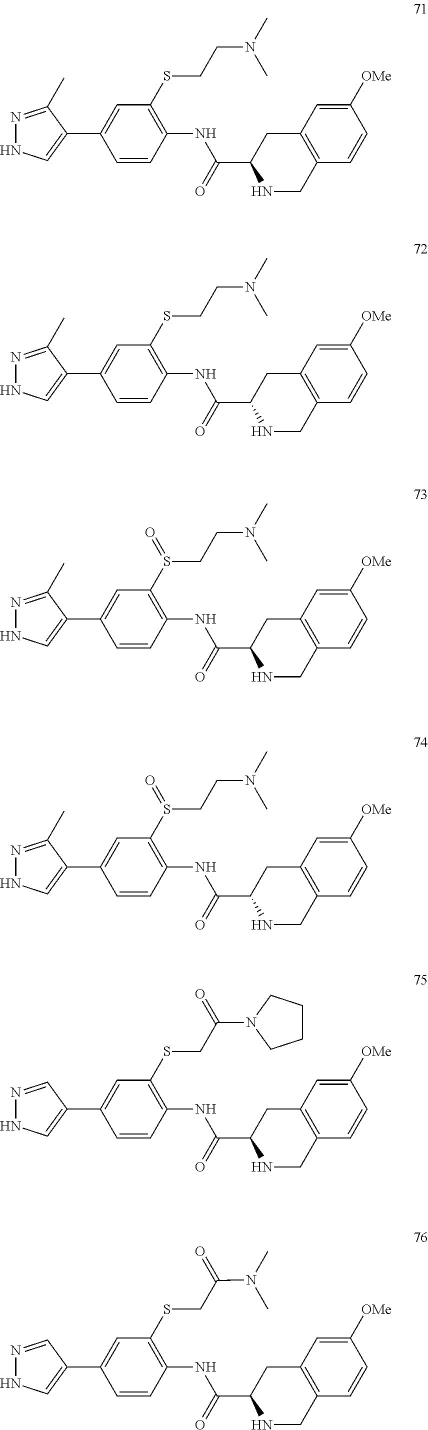 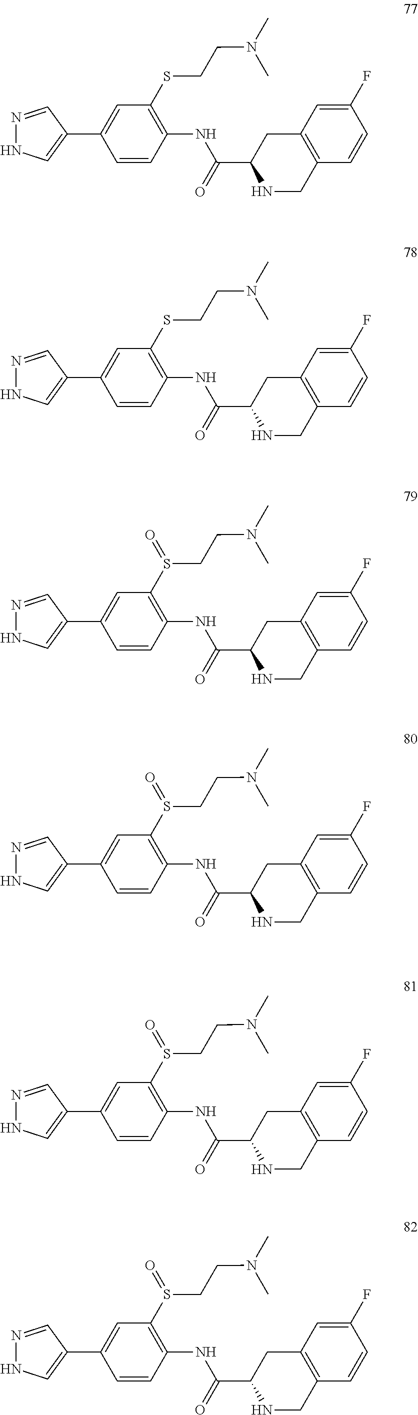 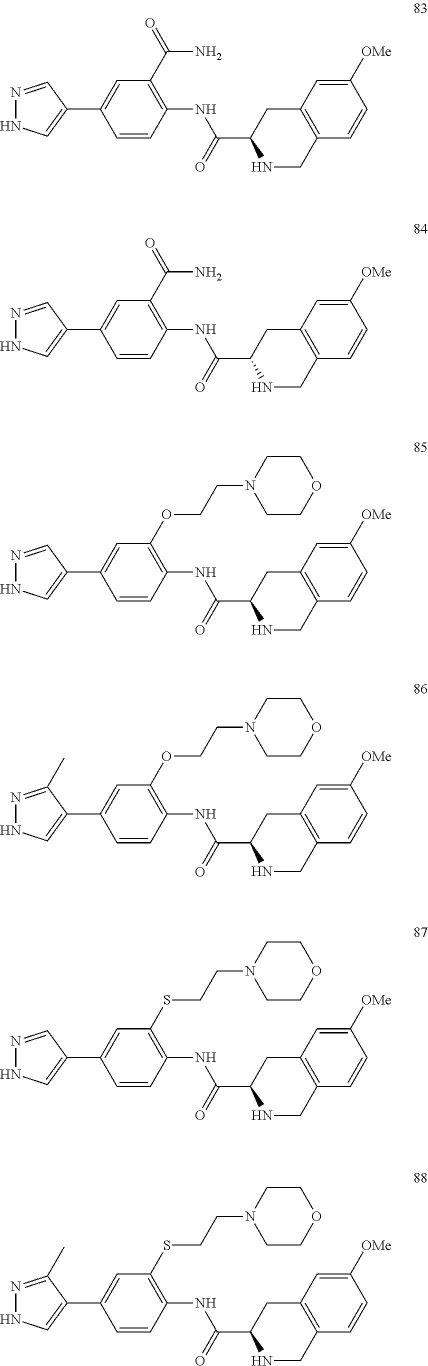 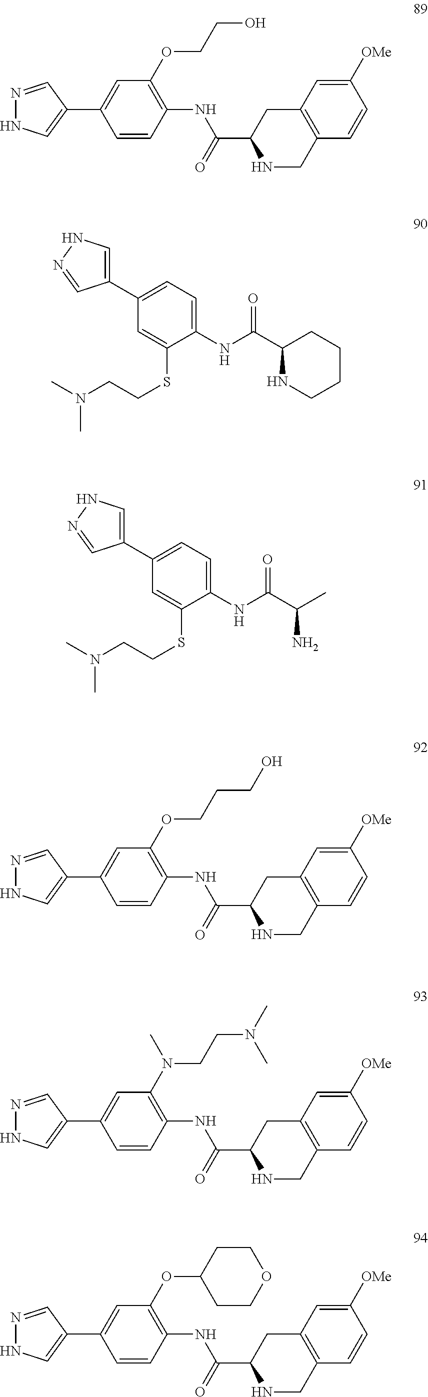 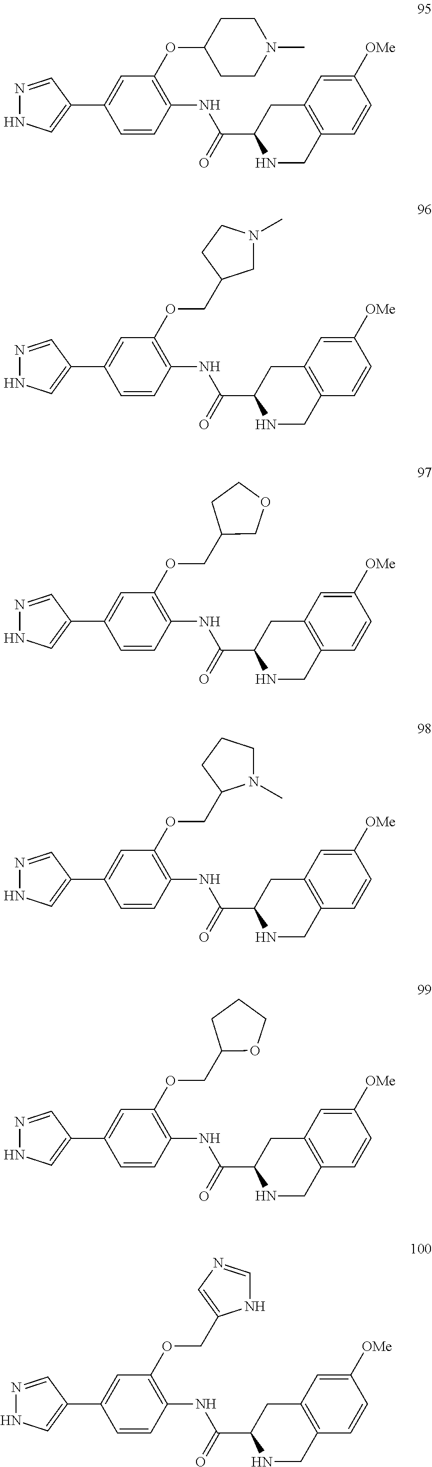 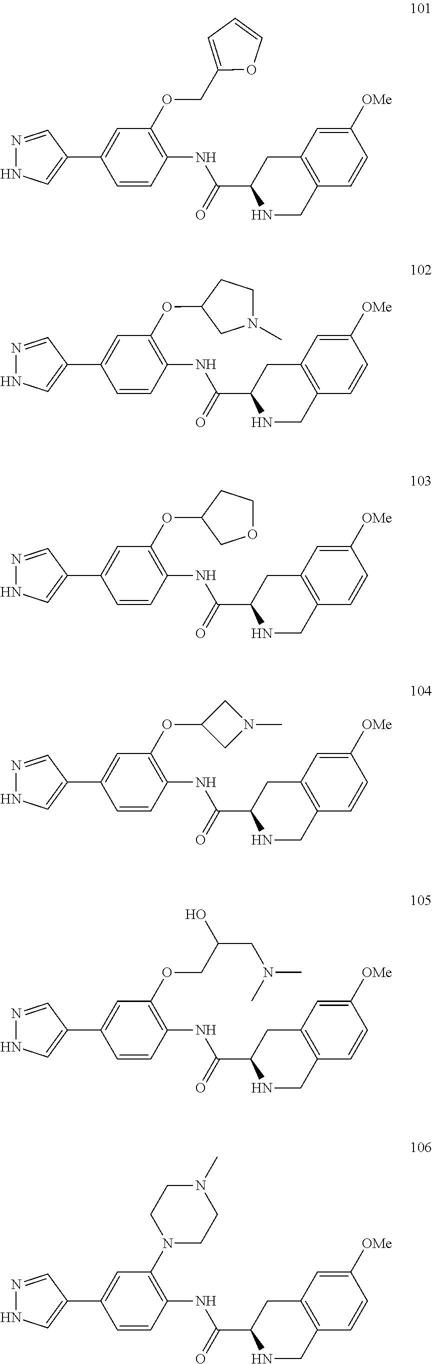 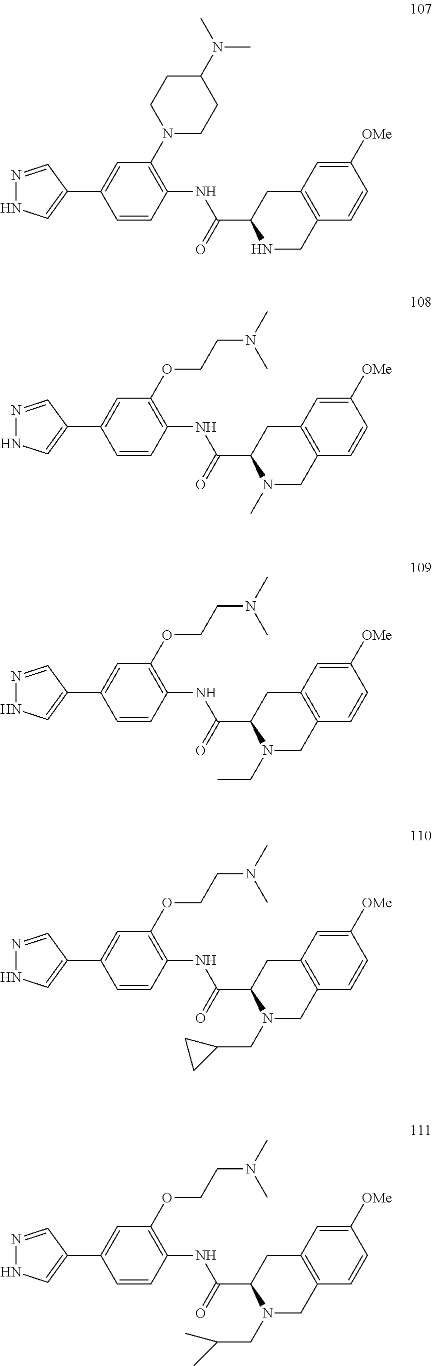 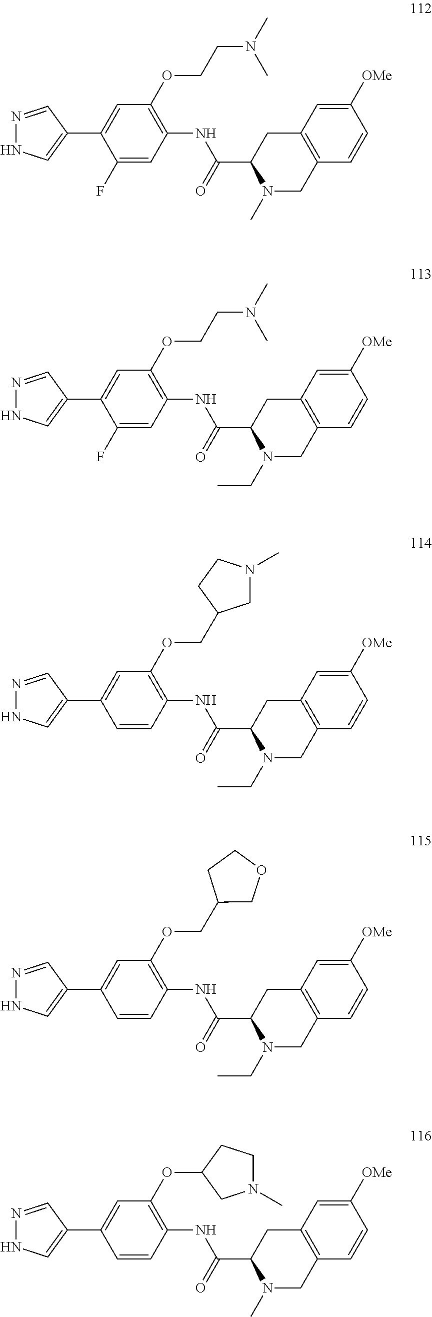 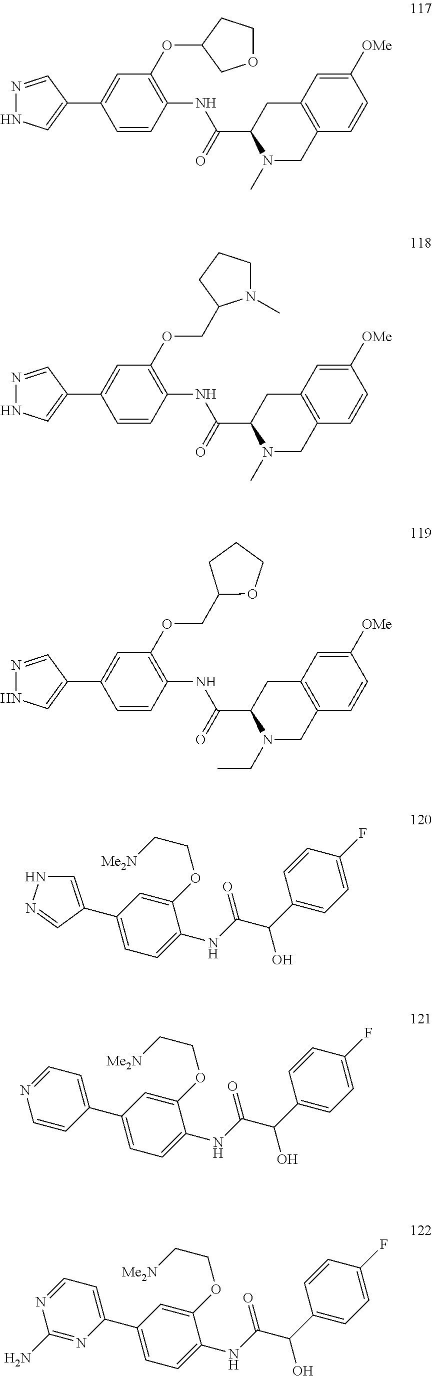 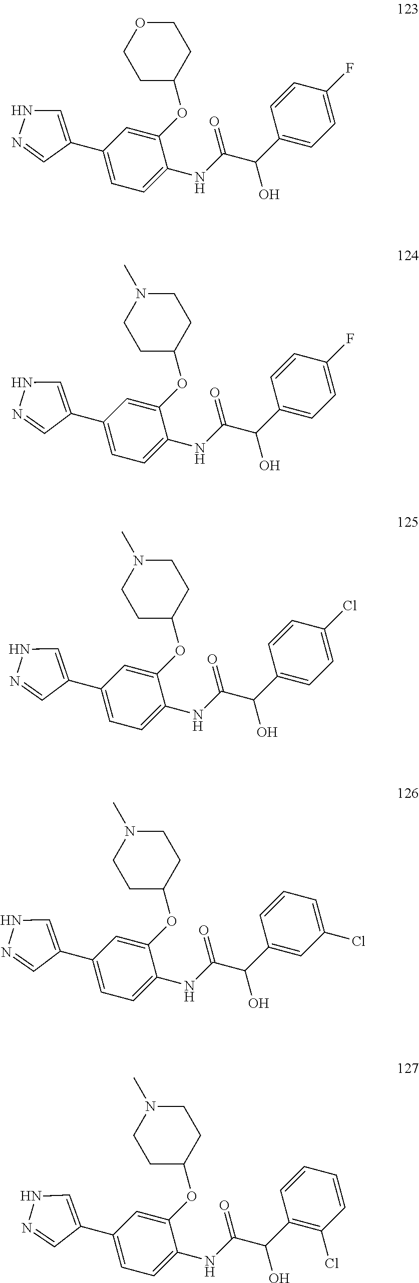 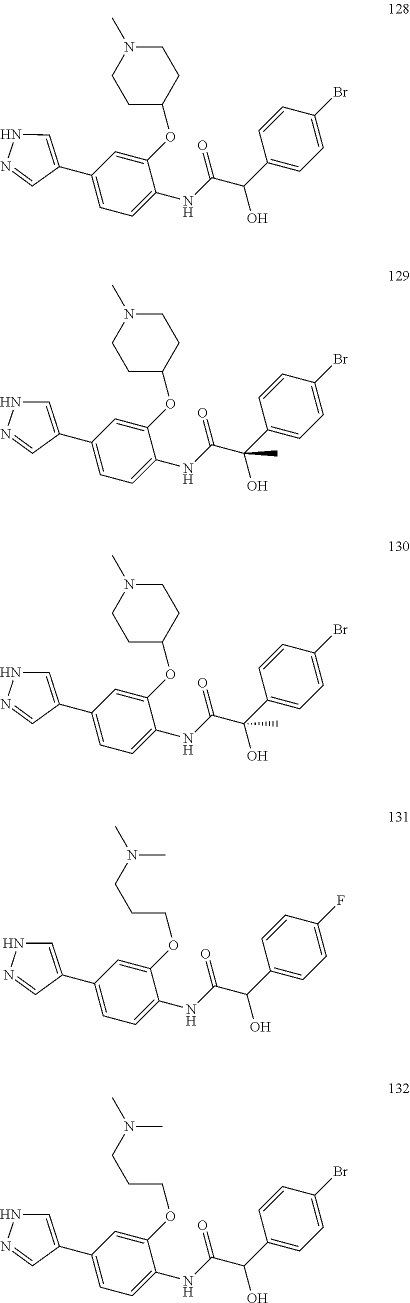 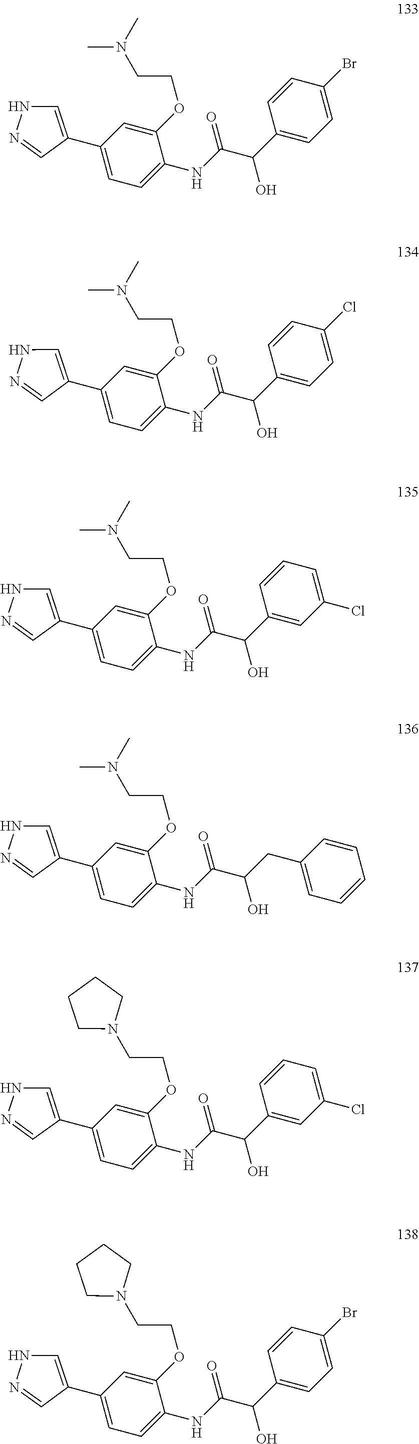 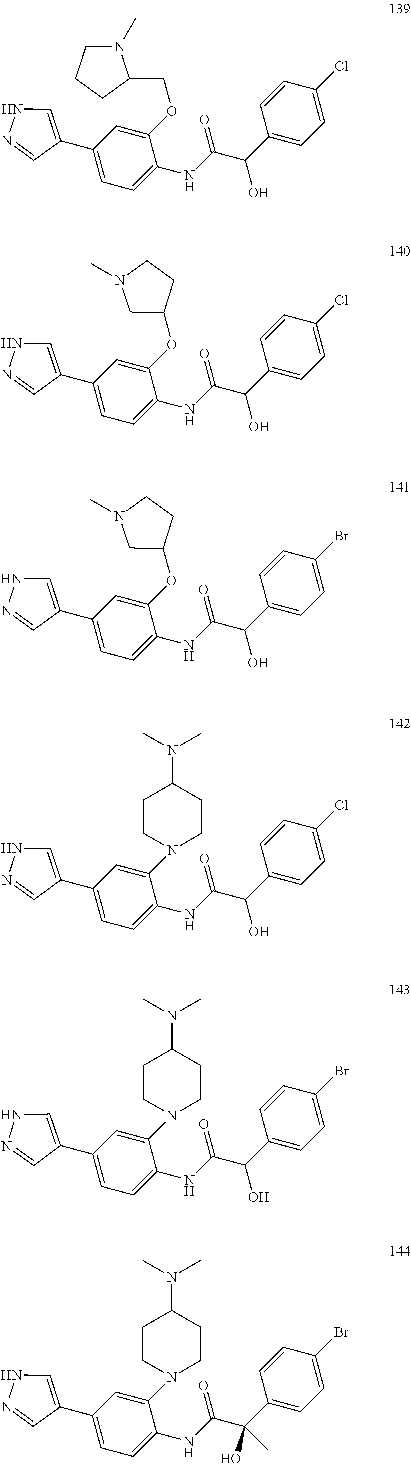 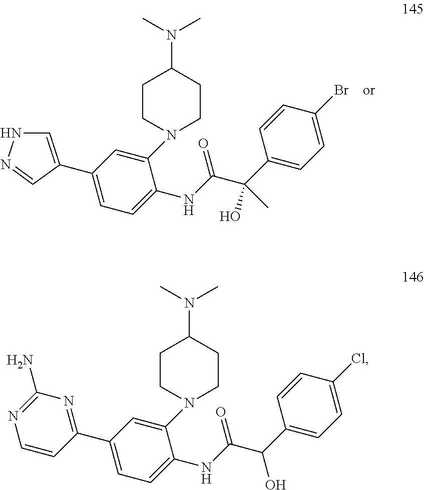
or any salt, stereoisomer, tautomer, hydrate, solvate, or prodrug thereof.

13. A pharmaceutical composition comprising a compound of claim 1 and a pharmaceutically acceptable excipient.

14. A pharmaceutical combination comprising a compound of claim 1 and an effective amount of a second medicament.

15.-24. (canceled)

25. A pharmaceutical composition comprising the combination of claim 14 and a suitable excipient.

26. A method of treatment of a malcondition in a patient in need thereof, comprising administering a therapeutically effective amount of the compound of claim 1 to the patient at a frequency of administration and for a duration of time sufficient to provide a beneficial effect to the patient.

27. The method of claim 26 wherein the malcondition comprises cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

28. The method of claim 26 for which binding of a ligand to a Rho kinase or inhibition of a bioactivity of a Rho kinase, or both, is medically indicated.

29. A method of treatment of a malcondition in a patient, comprising administering to the patient the pharmaceutical combination of claim 14 in a therapeutically effective amount at a frequency of administration and for a duration of time sufficient to provide a beneficial effect to the patient.

30. The method of claim 29, wherein the malcondition comprises cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

31. The method of claim 30 for which binding of a ligand to a Rho kinase or inhibition of a bioactivity of a Rho kinase, or both, is medically indicated

32. The method of claim 26 further comprising administration of an effective amount of an additional medicament.

33. The method of claim 32 wherein the additional medicament comprises an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

34. The method of claim 33 wherein the anti-proliferative agent comprises an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate.

35. The method of claim 33 wherein the anti-glaucoma agent comprises a beta receptor-blocker, a prostaglandin, an alpha-adrenergic agonist, a parasympathomimetic, or a carbonic anhydrase inhibitor.

36. The method of claim 33 wherein the anti-hypertensive agent comprises a beta receptor-blocker, a calcium channel blocker, a diueretic, an angiotensin converting enzyme (ACE) inhibitor, a renin inhibitor, or an angiotensin receptor antagonist.

37. The method of claim 33 wherein the anti-atherosclerotic agent comprises a 3-HMG-coA-reductase inhibitor, a statin, atorvastatin, simvastatin, niacin, or a combination drug such as vytorin.

38. The method of claim 33 wherein the anti-multiple sclerosis agent comprises beta-inteferon, tysabri, or glatirimar acetate.

39. The method of claim 33 wherein the anti-angina agent comprises a beta receptor-blocker, a calcium channel blocker, nitroglycerin, isosoribide mononitrate, nicorandil, or ranolanzine.

40. The method of claim 33 wherein the anti-erectile dysfunction agent comprises a phosphodiesterase-5 inhibitor.

41. The method of claim 33 wherein the anti-stroke agent comprises tissue plasminogen activator.

42. The method of claim 33 wherein the anti-asthma agent comprises a bronchodilator, an inhaled corticosteroid, a leukotrine blockers, cromolyn, nedocromil, or theophylline.

43.-48. (canceled)
